(19) United States
(12) Patent Application Publication
(10) Pub. No.: US 2025/0213670 A1
(43) Pub. Date: Jul. 3, 2025

(54) MULTIVALENT MOPEVAC-BASED IMMUNOGENIC COMPOSITION FOR VACCINATION AGAINST NEW WORLD ARENAVIRUSES AND THERAPEUTIC USE(S) THEREOF

(71) Applicant: INSTITUT PASTEUR, PARIS CEDEX 15 (FR)

(72) Inventors: Sylvain BAIZE, PARIS (FR); Xavier CARNEC, PARIS (FR); Stephanie REYNARD, PARIS (FR)

(21) Appl. No.: 18/844,288

(22) PCT Filed: Mar. 15, 2023
(86) PCT No.: PCT/EP2023/056673

§ 371 Date: Sep. 5, 2024

(30) Foreign Application Priority Data
Mar. 15, 2022 (EP) 22305301.8

(51) Int. Cl. A61K 39/12; A61K 39/00; A61P 31/14; C12N 7/00

(52) U.S. Cl. CPC A61K 39/12; A61P 31/14; C12N 7/00; A61K 2039/5254; A61K 2039/5256; A61K 2039/545; A61K 2039/575; A61K 2039/70; C12N 2760/10022; C12N 2760/10034

(57) ABSTRACT

The invention concerns a multivalent immunogenic composition comprising recombinant live attenuated Mopeia viruses (MOPV), wherein each valence is constituted by a recombinant live attenuated Mopeia virus in which the MOPV nucleoprotein (NP) has attenuated exonuclease activity and the encoded glycoprotein precursor (GPC) is from a New World arenavirus selected from one of the following arenaviruses: Machupo virus (MACV), Sabia virus (SABV), Chapare virus (CHAPV), Junin virus (JUNV) and Guanarito virus (GTOV). The invention also concerns a combination of active ingredients, a composition or vaccine, or a therapeutically effective composition, comprising such recombinant live attenuated Mopeia viruses (MOPV) for use in eliciting a protective immune response in a mammalian host against a New World arenavirus infection. The invention also concerns a method of preparing such recombinant live attenuated Mopeia viruses (MOPV) in a eukaryotic host cell and a method of preparing a multivalent, in particular a pentavalent, immunogenic composition comprising recombinant live attenuated Mopeia viruses (MOPV) expressing a GPC protein of a New World arenavirus selected among: Machupo virus (MACV), Sabia virus (SABV), Chapare virus (CHAPV), Junin virus (JUNV) and Guanarito virus (GTOV).

The present invention pertains to the field of immunogenic compositions and vaccines, and their use in the fight against New World arenaviruses.

The invention relates to a multivalent immunogenic composition comprising recombinant live attenuated Mopeia viruses (MOPV), each recombinant live attenuated Mopeia virus being based on a so-called MOPEVAC vector encoding a non-MOPV New World arenavirus glycoprotein precursor (GPC). In particular, the invention relates to a pentavalent immunogenic composition where the said GPC are selected from the following arenaviruses: Machupo virus (MACV), Sabia virus (SABV), Chapare virus (CHAPV), Junin virus (JUNV) and Guanarito virus (GTOV).

The invention also relates to means for use for eliciting a protective, and preferentially prophylactic, immune response, especially against New World arenaviruses infection(s) and New World arenaviruses-caused diseases, such as hemorrhagic fevers caused by New World arenaviruses or other symptoms and consequences of New World arenaviruses infection(s). The invention also encompasses means for use for therapeutic treatment against New World arenaviruses infection(s), symptom(s) or disease(s) caused by New World arenaviruses infection(s).

The invention also relates to a method of preparing a recombinant live attenuated Mopeia virus (MOPV) in a eukaryotic host cell, said recombinant live attenuated Mopeia virus (MOPV) comprising an heterologous nucleic acid encoding a non-MOPV New World arenavirus GPC from an arenavirus selected among: Machupo virus (MACV), Sabia virus (SABV), Chapare virus (CHAPV), Junin virus (JUNV) and Guanarito virus (GTOV). The invention also relates to a method of preparing a multivalent immunogenic composition starting from the products obtained from the method of preparing a recombinant live attenuated Mopeia virus (MOPV) as described herein.

It is known from WO2017/068190 a vaccine against the Lassa virus, a so-called Old World arenavirus, the vaccine being based on an live attenuated Mopeia virus (MOPV)—which is also an Old World arenavirus. In this so-called MOPEVAC_{LASV} vaccine, the attenuation is provided by mutations into the exonuclease domain of the nucleoprotein (NP) of the MOPV virus, and allows the recombinant virus to be much more immunogenic and safe. In the MOPEVAC_{LASV} vaccine, the glycoprotein precursor (GPC) of the Lassa virus replaces the GPC of MOPV in order to induce immunity against the Lassa pathogenic arenavirus (LASV). The efficiency of the MOPEVAC_{LASV} vaccine, carrying the GPC of Lassa virus (LASV), has been tested in cynomolgus monkeys. The monkeys were vaccinated with a single injection and challenged with LASV. The inoculation of the vaccine did not cause any clinical sign or fever. After challenge, the four animals that received the vaccine experienced only a transient fever for three over four animals, the fourth remaining totally healthy, and they all developed T-cell responses against the Lassa virus and specific IgG and neutralizing antibodies (1). The control animals that had received no vaccine before challenge experienced severe disease leading to their euthanasia before the end of the experiment.

The recent emergence of huge epidemics like ebola virus or pandemics like SARS-COV-2 have highlighted the importance for preparedness against viruses with high potential to become epidemic. Arenaviruses are considered to be at risk of emergence. The constant expansion of the area of distribution of the reservoirs of New World arenaviruses provides an argument to consider seriously that risk (11).

New World arenaviruses are genetically distinct from the Old World arenaviruses to which the LASV or MOPV belong (7-9). However, it has also been provided evidence that MOPEVAC viruses carrying the GPC of New World arenaviruses can be generated (5). Carnec, X. et al., 2018 (5) and Mateo, M et al., 2019 [Science Transl Med, 2019] (1), describe that the MOPEVAC_{LASV} vaccine, i.e., a vaccine against an Old World arenavirus, is a safe, efficient. and highly immunogenic virus, able to induce high neutralizing antibody titers against the Lassa virus (LASV). Carnec, X. et al., 2018 (5) further describes the use of the MOPEVAC backbone with the GPC of several New World arenaviruses, i.e., the Machupo virus (MACV), Guanarito virus (GTOV), Chapare virus (CHAPV) and Sabia virus (SABV) to produce isolated MOPEVAC constructions, but no immunization assays are reported.

Five New World arenaviruses from South America are highly pathogenic and have to be handled in BSL4 laboratories: Machupo virus (MACV), Sabia virus (SABV), Chapare virus (CHAPV), Junin virus (JUNV) and Guanarito virus (GTOV). Except for JUNV that is endemic in Argentina, no licensed vaccine is available but these viruses cause epidemics and/or are an increasing threat for public health. Yet, developing a vaccine for each of these viruses would not be economically relevant.

In parallel, the literature provides evidence that neutralizing antibodies may be of crucial importance in the control of New World arenaviruses (2, 4, 10).

Present invention arises from experiments where inventors first tested the ability of a MOPEVAC_{MACV} construct to protect cynomolgus monkeys against a lethal challenge with Machupo virus (MACV). The vaccine was fully efficient in a prime only or in a prime boost strategy as no clinical sign was observed after challenge in vaccinated animals and a sterilizing immunity was achieved. The inventors then developed the multivalent vaccine termed MOPEVAC_{NEW} herein, a combination of five distinct MOPEVAC viruses, each carrying a different GPC from originating from one of the five pathogenic viruses that are the Machupo virus (MACV), Sabia virus (SABV), Chapare virus (CHAPV), Junin virus (JUNV) and the Guanarito virus (GTOV).

There is currently no available vaccine for any of Machupo virus (MACV), Sabia virus (SABV), Chapare virus (CHAPV) and Guanarito virus (GTOV). A need therefore remains regarding those specific pathogens, but more broadly, the experiments presented herein provide evidence that the MOPEVAC platform can be used in a multivalent way, especially in the pentavalent configuration described herein, against New World arenaviruses, in a very efficient way. Strikingly, the use in parallel of several valences, i.e., the concomitant use of a valence of each virus as described herein, was at risk of perturbating the efficiency of the administered pentavalent by comparison to administration of monovalent vaccines, and at risk to favor deleterious effects, notably deleterious effects that may arise because of antibody-dependent enhancement, noting the close proximity of the New World arenaviruses present in the multivalent composition of the invention.

Furthermore, in light of the obtained immune responses, as presented herein, present invention therefore also paves the way to a solution to the problem of providing a vaccination strategy enabling the handling of New World arenaviruses infections or epidemics considered more broadly, despite the fact that targeted viruses may diverge from the prototypic Old World MOPV or from the yet known New World arenaviruses.

The invention relates to a multivalent immunogenic composition comprising recombinant live attenuated Mopeia viruses (MOPV), wherein each valence is constituted by a recombinant live attenuated Mopeia virus wherein the expressed nucleoprotein (NP) and glycoprotein precursor (GPC) are encoded by the viral genome wherein:
a. the nucleic acid of the S segment encodes a MOPV nucleoprotein (NP) having attenuated exonuclease activity, and
b. the nucleic acid of the S segment is deleted for the ORF of the glycoprotein precursor (GPC) of the Mopeia virus and comprises a heterologous nucleic acid encoding a New World arenavirus glycoprotein precursor (GPC) from one of the following arenaviruses: Machupo virus (MACV), Sabia virus (SABV), Chapare virus (CHAPV), Junin virus (JUNV) and Guanarito virus (GTOV).

According to a particular embodiment, the immunogenic composition comprises at least five different recombinant live attenuated Mopeia viruses (MOPV), whose expressed GPC are those of the five different New World GPC arenaviruses described above, i.e., the multivalent immunogenic composition can embed more than five different recombinant live attenuated Mopeia viruses (MOPV). As a synonym, it can also be said that an immunogenic composition of the invention is a multivalent composition and comprises a plurality, i.e., at least five, of different types of recombinant live attenuated Mopeia viruses (MOPV), according to the definitions provided herein. In a particular embodiment, the immunogenic composition contains five different recombinant live attenuated Mopeia viruses (MOPV) and is accordingly a pentavalent immunogenic composition. Differently said and according to a particular embodiment, at least the above-mentioned five different New World GPC arenavirus glycoproteins are expressed in the multivalent immunogenic composition. According to a particular embodiment, the above-mentioned five different New World GPC arenavirus glycoproteins are expressed in a pentavalent immunogenic composition.

According to the invention, a “recombinant attenuated Mopeia virus (MOPV)” is a recombinant live attenuated Mopeia virus.

By “heterologous nucleic acid” it is meant that a nucleic acid molecule, which does not originate from a MOPV arenavirus (i.e., a non-MOPV nucleic acid molecule) is inserted e.g., cloned, into a genomic segment of a MOPV arenavirus. According to the invention, the heterologous nucleic acid is a nucleic acid molecule from the genome of a New World arenavirus that is inserted into the scaffold of a live attenuated Mopeia virus (MOPV) as a replacement sequence to give rise to a “recombinant live attenuated Mopeia virus (MOPV)” as described herein. According to the invention, the heterologous nucleic acid can encode a New World arenavirus GPC. According to an embodiment, the heterologous nucleic acid encodes the ORF (Open Reading frame) of a GPC of a New World arenavirus.

According to a particular embodiment, the heterologous nucleic acid is cloned in a plasmid, and the plasmid bears the control sequences such as a promoter and/or a terminator suitable for expression of the nucleic acid in a host cell. An extra non templated-G base can be included at the beginning of the cloned sequence, i.e., the heterologous nucleic acid, for a correct transcription and replication of the viral segments drived by the plasmid (see for instance, the Material and Methods section “Plasmids” of WO2017/068190, and/or the material and methods section of Carnec, X. et al. A Vaccine Platform against Arenaviruses Based on a Recombinant Hyperattenuated Mopeia Virus Expressing Heterologous Glycoproteins. J. Virol. 92, (2018) (5)).

In a particular embodiment, the heterologous nucleic acid is an expression cassette, i.e., contains in addition to the ORF, the expression control sequences including a promoter and a terminator suitable for expression of the nucleic acid in a host cell.

Examples of New World arenaviruses include: Amapari virus, Chapare virus, Flexal virus, Guanarito virus, Junin virus, Latino virus, Machupo virus, Oliveros virus, Parana virus, Patawa virus, Pichinde virus, Pirital virus, Sabia virus, Tacaribe virus, Tamiami virus, and Whitewater Arroyo virus. Whitewater Arroyo virus is a pathogenic New World arenavirus.

Description of New World arenaviruses can readily be found in the literature (see, as non limitative examples, (12) and (13)). In a particular embodiment, the New World arenaviruses are from clade A, B, C or D, or any combination or recombination thereof, in particular, the New World arenaviruses are from clades B and D. In a more specific embodiment, the New World arenaviruses are from clade B. For instance, Machupo virus (MACV), Sabia virus (SABV), Chapare virus (CHAPV), Junin virus (JUNV) and Guanarito virus (GTOV) all pertain to the so-called New World arenaviruses clade B. Whitewater Arroyo (WWWAV) virus comes from a recombination between viruses originating from clade A and clade B, and the literature classifies it in clade D, according to some authors (see Table 1 of (12), “tentative”).

In a particular embodiment of the invention, the immunogenic composition is a pentavalent immunogenic composition with heterologous nucleic acids each encoding one New World arenavirus glycoprotein precursor (GPC) of: Machupo virus (MACV), Sabia virus (SABV), Chapare virus (CHAPV), Junin virus (JUNV) and Guanarito virus (GTOV).

By “nucleic acid encoding a MOPV nucleoprotein (NP) having attenuated exonuclease activity”, it is meant herein a nucleic acid molecule comprising the ORF for the nucleoprotein of the Mopeia virus wherein said ORF comprises codon mutation(s) with respect to the ORF of the wild-type Mopeia virus, in order to express a nucleoprotein that has attenuated exonuclease activity (also designated in the art as exoribonuclease activity). Mutations in the codons encompass mutations in at least two codons, in particular of 2, 3, 4, 5 or 6 codons wherein the mutation(s) collectively result in the impairment, especially the attenuation or the suppression of the exonuclease activity of the encoded mutated nucleoprotein with respect to the wild type nucleoprotein of MOPV. These mutations are especially described herein in relation to the mutated amino acid residues of the wild type nucleoprotein of a Mopeia virus and can be deduced from the substituting amino acid residues.

The scaffold for a “recombinant live attenuated Mopeia virus (MOPV)” is thoroughly described in WO2017/068190, which is referred to herein, and incorporated by reference in its entirety in present application, and in Carnec, X. et al. A Vaccine Platform against Arenaviruses Based on a Recombinant Hyperattenuated Mopeia Virus Expressing Heterologous Glycoproteins. J. Virol. 92, (2018) (5). According to said references, a “recombinant live attenuated Mopeia virus (MOPV)”, is a live attenuated Mopeia virus (MOPV) wherein exonuclease activity has been impaired or suppressed, and especially contains a MOPV nucleoprotein (NP) having attenuated exonuclease activity.

A “recombinant live attenuated Mopeia virus (MOPV)” is used in instant invention as a vector for expression of antigenic polypeptide of arenaviruses classified as “New World arenaviruses”. In a particular embodiment, the New World arenaviruses are classified in New World arenaviruses clades B and D. In a more specific embodiment, the New World arenaviruses are from clade B.

As used herein, the expression “recombinant live attenuated Mopeia virus” is used interchangeably with “recombinant live attenuated MOPV”, “recombinant live attenuated Mopeia virus particles”, “MOPEVAC_{NEW}” or “recombinant MOPV vector”.

To provide the recombinant live attenuated Mopeia virus of the invention, a Mopeia virus has been used wherein the S segment of its genome, has been recombined so that it can express an antigenic polypeptide of at least one New World arenaviruses, i.e., an antigenic polypeptide which is a glycoprotein precursor (GPC) of the envelope glycoproteins (GP1 and GP2) of New World arenaviruses as described herein, and which further encodes a mutated nucleoprotein of the Mopeia virus. According to a particular embodiment, the constructions described herein enable the resulting viruses to exhibit the capability to be poorly replicative in immune cells and/or to be able to activate, especially strongly activate, dendritic cells (DC) and macrophages (MP). According to a particular embodiment, the resulting viruses are more immunogenic than the departure wild type counterpart. In particular, the recombinant live attenuated MOPV viruses advantageously have no pathogenic phenotype.

As used herein, the expression “nucleoprotein (NP)” in the context of the recombinant live attenuated Mopeia virus of the invention designates a nucleoprotein that is mutated with respect to the wild type nucleoprotein of the MOPV strain AN21366 (SEQ ID NO: 1, GenBank accession number: AEO89356.1) by substitution of at least two, in particular substitution of 2, 3, 4, 5 or 6 amino acid residues wherein the mutation(s) collectively result in the impairment, especially the attenuation or the suppression, of the exonuclease activity of the wild type nucleoprotein of MOPV (paralleling the disclosure of section “C. Attenuation of MOPV” of WO2017/068190). The mutations introduced in the MOPV nucleoprotein enabled to generate an attenuated MOPV, as described in WO2017/068190. The invention makes use of recombinant attenuated MOPV able to replicate in a host to an extent that is sufficient for inducing an immune response but that is not sufficient for inducing a disease.

It is acknowledged that the exonuclease activity of the wildtype NP of the MOPV strain AN21366 allows for an escape to the IFN response of an infected individual. More details in this respect are provided in Example 2 of WO2017/068190. According to the invention, a Mopeia virus (MOPV) is said to be attenuated through an impairment, especially an attenuation or a suppression, of the exonuclease activity of the wild-type nucleoprotein of MOPV, if its NP is mutated with respect to the wild-type NP of the MOPV strain AN21366 (which wild-type NP is, according to a particular embodiment transposable throughout present description, represented by SEQ ID NO: 1) and the mutation(s) destabilize(s) and/or abolish(es) the exonuclease activity of the wild type NP of the MOPV strain AN21366 (which is, according to a particular embodiment transposable throughout present description, represented by SEQ ID NO: 1). In other words, the attenuation through mutation(s) of the MOPV used in instant invention causes a loss of function of the NP of the MOPV described and discussed herein with respect to the NP of the MOPV strain AN21366 (which is, according to a particular embodiment transposable throughout present description, represented by SEQ ID NO: 1), when found in a virus, and said loss of function can readily be determined through an appropriate experimental set-up, according to thorough guidance known in the art and thus available to the skilled person, or provided in the literature, especially in WO2017/068190.

According to particular embodiments, a loss of function of the exonuclease activity of the NP with respect to the exonuclease activity of the NP of the MOPV strain AN21366 can reach an extent of (minus) 50%, 60%, 70%, 80%, 90% or 100% with respect to a reference value, as comparatively measured through an appropriate experimental set-up, for example a reporter gene assay such as disclosed in Example 6 of WO2017/068190 (and FIG. 7 of WO2017/068190). According to such an exemplary reporter gene assay, the IFN-antagonist activity of tested NP mutants was measured. In a particular implementation of such an exemplary assay, cells transfected with a plasmid encoding an IRF-3-promoter driven luciferase and a plasmid encoding wild type (wt) or mutant forms of NP can be infected with the Sendai virus (SeV), a strong inducer of IRF-3 and IFN responses. Then, the induction of the IFN-derived promoter by Sendai virus (SeV) can be assessed in transfected cells expressing different NP mutants. FIG. 7B of WO2017/068190 demonstrate that in the context of WO2017/068190, NP-wt could block the induction of the luciferase expression in response to SeV. On the contrary, all the tested mutants of WO2017/068190, mutated in the so-called ExoN domain, were affected in their ability to reduce induction of the reporter gene expression. Accordingly, by comparing the extent of modulation of the observed signal (in the case of an assay as disclosed in Example 6 and FIG. 7 of WO2017/068190, the signal can correspond to an absolute value or to a fold change in IRF3 induction, if necessary by reference to the induction observed for SeV), the skilled person can readily determine whether the loss of function of the exonuclease activity of the NP of a particular mutant, with respect to the exonuclease activity of the NP of the MOPV strain AN21366 reaches an extent of (minus) 50%, 60%, 70%, 80%, 90% or 100% as defined herein for assessing the presence of a loss of function of the exonuclease activity of the NP of a MOPV.

The suitability of the mutations leading to a loss of function mutations can therefore readily be determined by the skilled person. The domain responsible for the exonuclease activity of the wild type NP of the MOPV strain AN21366 is located between residues 340 and 570 of SEQ ID NO: 1, GenBank accession number: AEO89356.1. Any part of this domain can be mutated so as to destabilize and/or abolish the exonuclease activity of the wild type NP of the MOPV strain AN21366, in particular with a loss of function of the exonuclease activity of the NP with respect to the exonuclease activity of the NP of the MOPV strain AN21366 can reach an extent of (minus) 50%, 60%, 70%, 80%, 90% or 100% with respect to a reference value, as described herein.

According to a particular, specific, aspect, residues 390, 392, 393, 430, 467, 529 et 534 of SEQ ID NO: 1, which are in the domain responsible for the exonuclease activity of the wild type NP of the MOPV strain AN21366, are specifically known to be involved in the exonuclease activity (ExoN) of MOPV. Accordingly, such residues may be targeted by mutations, being understood that mutations of residue(s) around residues 390, 392, 393, 430, 467, 529 and 534 of SEQ ID NO: 1, and/or residue(s) between residues 340 and 570 of SEQ ID NO: 1 can also be suited as loss of function mutations, as defined herein.

According to a particular embodiment, the amino acid positions D390 and G393 of the MOPV nucleoprotein are substituted to attenuate the exonuclease function of the nucleoprotein (NP). In a specific embodiment, the amino acid substitutions are D390A and G393A (MOPV-ExoN in WO2017/068190). The specific mutations disclosed herein are identified by reference to the position of the amino acid residues in the sequence of the nucleoprotein of the Mopeia strain AN21366 (GenBank accession numbers JN561684.1 (S segment—that includes the MOPV nucleoprotein sequence AEO89356.1), referred to as SEQ ID NO: 2 herein and JN561685.1 (L segment) referred to as SEQ ID NO: 3 herein, when polynucleotide sequences are considered—correspondence with the polypeptide sequence can readily be done using the annotated sequences of the databases, the polypeptide sequence of the NP of the wild type Mopeia strain AN21366 being also provided as SEQ ID NO: 1). If a different strain of Mopeia virus is used according to the invention, the amino acid residues may easily be determined by alignment of the amino acid sequence with the NP sequence of the Mopeia strain AN21366.

According to other embodiments, which can be cumulated to the embodiment above, or according to any combinations of possible substitutions, at least one further amino acid substitution is added at a position selected from E392, H430, D467, H529, and D534 of the MOPV nucleoprotein. In specific embodiments, the further substitution is selected from E392A, H430A, D467A, H529A, and D534A or any combination thereof.

In some embodiments of the recombinant live attenuated MOPV, the nucleoprotein comprises an amino acid substitution at amino acid position D390 or G393. In some embodiments of the recombinant attenuated MOPV, the nucleoprotein comprises an amino acid substitution at amino acid position D390 or G393, and further comprises at least one amino acid substitution at a position selected from E392, H430, D467, H529, and D534. In some embodiments of the recombinant attenuated MOPV, the nucleoprotein comprises amino acid substitutions at amino acid positions D390 and G393. In some embodiments of the recombinant attenuated MOPV, the nucleoprotein comprises amino acid substitutions at amino acid positions D390 and G393, and further comprises at least one amino acid substitution at a position selected from E392, H430, D467, H529, and D534.

In some embodiments of the recombinant live attenuated MOPV, the nucleoprotein comprises a D390A or G393A amino acid substitution, in particular with respect to SEQ ID NO: 1. In some embodiments of the recombinant attenuated MOPV, the nucleoprotein comprises D390A and G393A amino acid substitutions, in particular with respect to SEQ ID NO: 1. In some embodiments of the recombinant attenuated MOPV, the nucleoprotein further comprises at least one amino acid substitution selected from E392A, H430A, D467A, H529A, and D534A, in particular with respect to SEQ ID NO: 1. In some embodiments, the recombinant attenuated MOPV comprises amino acid substitution D390A, G393A, E392A, H430A, D467A, H529A, and D534A, in particular with respect to SEQ ID NO: 1. The recombinant attenuated MOPV comprising amino acid substitutions at amino acid positions D390A, G393A, E392A, H430A, D467A, H529A, and D534A is named MOPV-ExoN enhanced in WO2017/068190.

According to a particular embodiment, the amino acid positions D390, H430 and D467 of the MOPV nucleoprotein are substituted, in particular with respect to SEQ ID NO: 1, to attenuate the exonuclease function of the nucleoprotein (NP). In a specific embodiment, the amino acid substitutions are D390A, H430A and D467A (See polynucleotide constructions described herein).

According to other embodiments, which can be cumulated to the embodiment above, or according to any combinations of possible substitutions, at least one further amino acid substitution is added at a position selected from E392, G393, H529, and D534 of the MOPV nucleoprotein, in particular with respect to SEQ ID NO: 1. In specific embodiments, the further substitution is selected from E392A, G393A, H529A, and D534A or any combination thereof.

In some embodiments of the recombinant live attenuated MOPV, the nucleoprotein comprises a D390A, a H430A or a D467A amino acid substitution (1 substitution), in particular with respect to SEQ ID NO: 1. In some embodiments of the recombinant attenuated MOPV, the nucleoprotein comprises a D390A, a H430A and a D467A amino acid substitutions (3 substitutions), in particular with respect to SEQ ID NO: 1. In some embodiments of the recombinant attenuated MOPV, the nucleoprotein further comprises at least one amino acid substitution selected from E392A, G393A, H529A, and D534A, in particular with respect to SEQ ID NO: 1. In some embodiments, the recombinant attenuated MOPV comprises amino acid substitution D390A, H430A, D467A, E392A, G393A, H529A, and D534A, in particular with respect to SEQ ID NO: 1.

Therefore, according to particular embodiments of the multivalent, in particular pentavalent, immunogenic composition of the invention:
a. the MOPV nucleoprotein (NP) encoded by the nucleic acid of the recombinant S segment described herein having attenuated exonuclease activity, comprises amino acid substitutions at positions D390 and G393 with respect to Mopeia strain AN21366, in particular with respect to SEQ ID NO: 1, or
b. The MOPV nucleoprotein (NP) encoded by the nucleic acid of the recombinant S segment described herein having attenuated exonuclease activity, comprises amino acid substitutions at positions D390 and G393 with respect to Mopeia strain AN21366, in particular with respect to SEQ ID NO: 1 and at least one further amino acid substitution at a position selected from E392, H430, D467, H529, and D534, with respect to the AN21366 strain, in particular with respect to SEQ ID NO: 1.

According to particular embodiments, the amino acid substitution is (are) D390A and/or is G393A, wherein said numbering is based upon the Mopeia strain AN21366, in particular with respect to SEQ ID NO: 1.

According to particular embodiments, the nucleoprotein further comprises at least one amino acid substitution selected from E392A, H430A, D467A, H529A, and D534A, in particular with respect to SEQ ID NO: 1, wherein said numbering is based upon the Mopeia strain AN21366, in particular with respect to SEQ ID NO: 1.

Therefore, according to particular embodiments of the multivalent, in particular pentavalent, immunogenic composition of the invention:
a. the MOPV nucleoprotein (NP) encoded by the nucleic acid of the recombinant S segment described herein having attenuated exonuclease activity, comprises amino acid substitutions at positions D390, H430 and D467 with respect to Mopeia strain AN21366, in particular with respect to SEQ ID NO: 1, or
b. the MOPV nucleoprotein (NP) encoded by the nucleic acid of the recombinant S segment described herein having attenuated exonuclease activity, comprises amino acid substitutions at positions D390, H430 and D467 with respect to Mopeia strain AN21366, in particular with respect to SEQ ID NO: 1, and at least one further amino acid substitution at a position selected from E392, G393, H529, and D534, with respect to the AN21366 strain, in particular with respect to SEQ ID NO: 1.

According to particular embodiments, the amino acid substitution is (are) D390A and/or H430A and/or D467A, wherein said numbering is based upon the Mopeia strain AN21366, in particular with respect to SEQ ID NO: 1.

According to particular embodiments, the nucleoprotein further comprises at least one amino acid substitution selected from E392A, G393A, H529A, and D534A, wherein said numbering is based upon the Mopeia strain AN21366, in particular with respect to SEQ ID NO: 1.

When substitution(s) is (are) present, they are defined above with respect to the Mopeia strain AN21366. The remainder of the nucleic acid described herein of the Open Reading Frame (ORF) which encodes the MOPV nucleoprotein (NP) having attenuated exonuclease activity, discussed in the previous paragraphs, has a sequence that is the sequence of the Mopeia strain AN21366 or a sequence that has at least 80%, at least 85%, at least 90%, at least 91%, at least 92%, at least 93%, at least 94%, at least 95%, at least 96%, at least 97%, at least 98%, at least 99% identity with the corresponding (aligned) wild type sequence of the MOPV, in particular with the corresponding sequence in the MOPV strain AN21366 (Genbank accession numbers JN561684.1 (which includes the MOPV nucleoprotein sequence AEO89356.1, SEQ ID NO: 1) and JN561685.1; SEQ ID NO: 2 and SEQ ID NO: 3, respectively. Similarly, the mutated MOPV nucleoprotein (NP) discussed in the previous paragraphs has the amino-acid sequence of the Mopeia strain AN21366 apart the positions where substitution(s) is (are) present, or has a sequence that has at least 80%, at least 85%, at least 90%, at least 91%, at least 92%, at least 93%, at least 94%, at least 95%, at least 96%, at least 97%, at least 98%, at least 99% identity with the corresponding wild type sequence of the MOPV, in particular with the corresponding polypeptide sequence encoded by the MOPV strain AN21366, in particular with respect to SEQ ID NO: 1. Identity percentages can be calculated following the guidance provided later in present description, and according to the common knowledge of the skilled person in the field.

According to a particular embodiment, the mutation(s) of the NP of the MOPV of the invention carried out for attenuation of the MOPV, still makes the mutated NP capable of supporting viral transcription and replication, in order to produce recombinant viruses (production step).

According to particular embodiments, the amino acid sequence of the non-MOPV GPC encoded by the heterologous nucleic acids on the S segment of the genome of the recombinant live attenuated MOPV, found in a multivalent, especially pentavalent, immunogenic composition of the invention, is selected from the group of the sequences SEQ ID NO: 4 (Genbank access number AAT40451.1 (protein) from SEQ ID NO: 9, i.e., Genbank access number AY619643 (S segment) for the Machupo virus (MACV), SEQ ID NO: 5 (Genbank access number YP_089665.1 (protein) from SEQ ID NO: 10, i.e., Genbank access number NC_006317 (S segment) for the Sabia virus (SABV), SEQ ID NO: 6 (Genbank access number YP_001816782.1 (protein) from SEQ ID NO: 11, i.e., Genbank access number NC_010562 (S segment) for the Chapare virus (CHAPV), SEQ ID NO: 7 (Genbank access number ABI51601.1 (protein) from SEQ ID NO: 12, i.e., Genbank access number DQ854733 (S segment) for the Junin virus (JUNV) and SEQ ID NO: 8 (Genbank access number AAN05423.1 (protein) from SEQ ID NO: 13, i.e., Genbank access number AY129247 (S segment) for the Guanarito virus (GTOV), respectively.

The skilled person will appreciate that genomic sequences of the various arenaviruses, especially New World arenaviruses, as well as of the proteins encoded by these arenaviruses, are publicly available. They can be found, e.g., on the web site of the Virus Sequence Database (VSD) established and maintained by the Center for Immunology and Pathology, National Institute of Health, Korea Centers for Disease Control and Prevention.

According to a particular embodiment, a multivalent immunogenic composition of the invention is formulated free of adjuvant(s) of the immune response and/or immunostimulant component(s).

In certain embodiments however, the immunogenic composition can be administered in combination with an adjuvant. The term “adjuvant” refers to a compound that when administered in conjunction with or as part of a composition described herein augments, enhances and/or boosts the immune response to a recombinant live attenuated MOPV present in the multivalent immunogenic composition, but when the compound is administered alone does not generate an immune response to the recombinant live attenuated MOPV. Adjuvants can enhance an immune response by several mechanisms including, e.g., lymphocyte recruitment, stimulation of B and/or T cells, stimulation of macrophages, and stimulation of dendritic cells. Adjuvants are well known in the art and can include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 222021 1), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et ah, in Vaccine Design: The Subunit and Adjuvant Approach (eds. Powell Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et ah, N. Engl. J. Med. 336, 86-91 (1997)). According to a particular embodiment, a multivalent immunogenic composition of the invention is an immunogenic composition wherein in the composition, valences comprise the at least the five different recombinant live attenuated Mopeia viruses (MOPV) with GPC proteins of Machupo virus (MACV), Sabia virus (SABV), Chapare virus (CHAPV), Junin virus (JUNV) and Guanarito virus (GTOV).

According to a particular embodiment, the immunogenic composition further comprises another valence of a New world arenavirus as disclosed in present description, in particular a further valence of a New World arenaviruses from clades B or D.

According to a more particular embodiment, in particular applicable to the two above paragraphs, the quantity/dosage of the valences of the at least five different recombinant live attenuated Mopeia viruses present in the composition is different between them, in particular the quantity(ies)/dosage(s) are each different between them, or at least one, two, three or four viruses is (are) present in a quantity/dosage within the composition that differs from the quantity(ies)/dosage(s) of the other virus(es).

According to another more particular embodiment, all valences of the at least the five different recombinant live attenuated Mopeia viruses are present in the immunogenic composition at an equal dose.

According to a particular embodiment, a multivalent immunogenic composition of the invention is a pentavalent immunogenic composition wherein in the composition the five different recombinant live attenuated Mopeia viruses (MOPV) that express GPC of one of Machupo virus (MACV), Sabia virus (SABV), Chapare virus (CHAPV), Junin virus (JUNV) and Guanarito virus (GTOV), are present and provide the five valences present in the composition.

According to a more particular embodiment, in particular applicable to the above-paragraph, the quantity(ies)/dosage(s) of the valences of the five different recombinant live attenuated Mopeia viruses present in the pentavalent composition is (are) different between them, in particular are each different between them, or at least one, two, three or four viruses is (are) present in a quantity/dosage within the composition that differs from the quantity/dosage of the other virus(es).

According to another more particular embodiment, all valences of the five different recombinant live attenuated Mopeia viruses are present in the pentavalent immunogenic composition at an equal dose. According to a particular embodiment, a multivalent immunogenic composition of the invention is a composition dosed between 1.10² and 1.10¹² ffu (Focus-forming units), or between 1.10³ and 1.10⁸ ffu, or in any range where the boundaries are selected from 1.10², 1.10³, 1.10⁴, 1.10⁵, 1.10⁶ and 1.10⁷ for the lower range, and selected from 1.10⁵, 1.10⁶, 1.10⁷, 1.10⁸, 1.10⁹, 1.10¹⁰, 1.10¹¹ and 1.10¹² for the higher range, in particular as measured by virus titration. According to a particular embodiment, a protocol as described in Carnec, X. et al. A Vaccine Platform against Arenaviruses Based on a Recombinant Hyperattenuated Mopeia Virus Expressing Heterologous Glycoproteins. J. Virol. 92, (2018) (5) for virus titration, can be used. However, the skilled person can appreciate that any protocol for virus titration, as commonly used in the field and described in the literature, may also be used. A particular protocol for viral titer calculation over a period of time of 7 days is provided in the Material and Methods section herein. According to a particular embodiment, the doses indicated above, according to any measure or range, are measured through the virus titration protocol set in the Material and Methods section herein, especially the described virus titration protocol over 7 days. Through virus titration, infectious particles are measured.

According to a particular embodiment, a multivalent immunogenic composition of the invention is a composition dosed at 2.10⁶ ffu.

According to particular embodiments, the doses are given for the total of the cumulated valences of the at least five different recombinant live attenuated Mopeia viruses (MOPV), which are present in the composition, in particular, in a pentavalent immunogenic composition, the five recombinant live attenuated Mopeia viruses (MOPV) comprising the GPC of the Machupo virus (MACV), Sabia virus (SABV), Chapare virus (CHAPV), Junin virus (JUNV) and Guanarito virus (GTOV).

According to a particular embodiment, a multivalent immunogenic composition of the invention is a pentavalent composition with the five recombinant live attenuated Mopeia viruses (MOPV) comprising the GPC of the Machupo virus (MACV), Sabia virus (SABV), Chapare virus (CHAPV), Junin virus (JUNV) and Guanarito virus (GTOV), each dosed between 1.10⁴ and 1.10⁶ ffu, in particular dosed at 4.10⁵ ffu.

According to a particular embodiment, the dose of the total of the cumulated valences of the at least five different recombinant live attenuated Mopeia viruses (MOPV), which are present in the composition, between 1.10² and 1.10¹² ffu (Focus-forming units), or between 1.10³ and 1.10⁸ ffu, or in any range where the boundaries are selected from 1.10², 1.10³, 1.10⁴, 1.10⁵, 1.10⁶ and 1.10⁷ for the lower range, and selected from 1.10⁵, 1.10⁶, 1.10⁷, 1.10⁸, 1.10⁹, 1.10¹⁰, 1.10¹¹ and 1.10¹² for the higher range, as measured by virus titration, in particular is 2.10⁶ ffu.

According to a particular embodiment however, the dose(s) indicated above are provided for a single valence of a recombinant live attenuated Mopeia viruses (MOPV) found within a composition as described herein. Such a dose may be in the range of 1.10² and 1.10⁷ ffu (Focus-forming units), or between 1.10³ and 1.10⁶ ffu, or in any range where the boundaries are selected from 1.10², 1.10³, 1.10⁴, 1.10⁵ and 1.10⁶ for the lower range, and selected from 1.10³, 1.10⁴, 1.10⁵, 1.10⁶ and 1.10⁷ for the higher range, as measured by virus titration.

According to a particular embodiment, the dose of a single valence of a recombinant live attenuated Mopeia viruses (MOPV) found within a composition as described herein is within any range where the boundaries are selected from 1.10², 1.10³, 1.10⁴, 1.10⁵ and 1.10⁶ for the lower range, and selected from 1.10³, 1.10⁴ and 1.10⁵ for the higher range, as measured by virus titration, in particular is between 1.10⁴ and 1.10⁶ ffu, in particular is within the range of 10⁵ ffu.

According to a particular embodiment, the dose of a single valence of a recombinant live attenuated Mopeia viruses (MOPV) found within a composition as described herein is as low as 1.10⁴, 1.10³ or 1.10² ffu.

Focus-forming units is a manner to measure the precise dose of a pathogen such a virus, i.e., a manner to quantify the number of viruses in a specific volume to determine the virus concentration (Virus quantification involves counting the number of viruses in said specific volume to determine the virus concentration), and specifically, to measure the dose of infectious particles within the volume. A common method to do so is a so-called “plaque assay”. Viral plaque assays determine the number of plaque forming units (pfu) in a virus sample, which is a manner to measure virus quantity. Such an assay is based on a microbiological method commonly conducted, for example, in petri dishes or multi-well plates. Pfu is obtained by counting discrete plaques (clear circular areas) on a lawn of cells, which are formed by the virus infecting and lysing the cell. The size of the plaque increases with time as the pathogen consumes more cells. Focus-forming units are an alternative manner to measure the precise dose of a pathogen using a so-called focus forming assay (FFA), which is a variation of the plaque assay. Instead of relying on cell lysis in order to detect plaque formation, the FFA employs immunostaining techniques using labelled, in particular fluorescently labelled, antibodies specific for a viral antigen to detect infected host cells and infectious virus particles before an actual plaque is formed. When a semi-solid culture medium is used in multi-well plates, the viruses that are present infect surrounding host cells but does not diffuse inside the well of the multi-well plate, so that the amount of infectious plaques that are counted correspond to the presence of an initial virus. A plaque therefore comes from the presence of one virus. Such methods are described in the literature and well-known to the skilled person in the field. It is to be understood that the skilled person can readily adapt a protocol for measuring Focus-forming units of viruses, to its needs, to reach the dosages described herein.

In a particular embodiment, a multivalent immunogenic composition provided herein is administered to a subject through a parenteral route of administration.

In some embodiments, a multivalent immunogenic composition provided herein is administered to a subject by, including but not limited to, oral, intradermal, intramuscular, intraperitoneal, intravenous, subcutaneous, percutaneous, intranasal and inhalation routes, and via scarification (scratching through the top layers of skin, e.g., using a bifurcated needle). In some embodiments, a subcutaneous or intravenous route is used. In some embodiments, the intramuscular route is used for administration.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and a suitable powder base such as lactose or starch.

The immunogenic composition(s) described herein may be formulated in unit dose forms, especially unit dose forms for parenteral administration, such as, for example, ampoules or vials, e.g., vials containing from about 10² to 10¹² focus forming units (ffu) or 10⁴ to 10¹⁴ physical particles for a single or for several cumulated valences of recombinant attenuated MOPV viruse(s), according to any embodiment described herein.

As skilled artisan will appreciate, the dosages of the multivalent immunogenic composition depend upon the type of vaccination and upon the subject, and their age, weight, individual condition, the individual pharmacokinetic data, and the mode of administration.

The invention accordingly also relates to a vaccine comprising a multivalent immunogenic composition of the invention as described in any embodiment disclosed herein, optionally with any one of: pharmaceutically acceptable carrier(s), delivery vehicle(s), excipient(s), preservative(s), or any combination thereof.

As defined herein, a “pharmaceutically acceptable carrier(s), delivery vehicle(s), excipient(s)” or “preservative(s)” encompass any substance that enables the formulation of a multivalent immunogenic composition, which makes it suited for administration to a human host and/or proper handling of a multivalent immunogenic composition for delivery to administration centres, respectively.

A carrier or delivery vehicle is any substance or combination of substances physiologically acceptable, i.e., appropriate for its use in a composition to be administered to a human, and thus non-toxic. Examples of such vehicles are phosphate buffered saline solutions, distilled water, emulsions such as oil/water emulsions, various types of wetting agents sterile solutions and the like. Examples of carriers, delivery vehicles, excipients or preservatives are commonly available to the skilled person.

The invention also relates to a combination of active ingredients comprising at least the five different recombinant live attenuated Mopeia viruses (MOPV) specifically described herein or in any part of present description, or a multivalent immunogenic composition comprising the said active ingredients as described in any embodiment or part of present description, for use in eliciting a protective immune response in a mammalian host, especially a human host, against a New World arenavirus infection, in particular a New World arenavirus infection selected from the group of a Machupo virus (MACV) infection, a Sabia virus (SABV) infection, a Chapare virus (CHAPV) infection, a Junin virus (JUNV) infection and a Guanarito virus (GTOV) infection,
wherein the active ingredients are administered as a single composition or as separate active ingredients, especially in the same administration step, and
wherein optionally the active ingredients or multivalent immunogenic composition are, within their respective compositions, either free of or associated with adjuvant(s), especially adjuvant(s) of the immune response and/or immunostimulant component(s),
and optionally wherein the active ingredients or multivalent immunogenic composition are, within their respective compositions, associated with pharmaceutically acceptable carrier(s), delivery vehicle(s), excipient(s), preservative(s), or any combination thereof.

Regarding the adjuvant(s), immunostimulant component(s), pharmaceutically acceptable carrier(s), delivery vehicle(s), excipient(s), preservative(s), reference is made to the description above.

By “protective immune response”, it is meant in that context that the active ingredient, in particular the immunogenic composition provides to the host or subject in need thereof a complete or partial protection against a subsequent challenge (or infection) with an arenavirus as defined herein, especially a New World arenavirus as described herein. In an embodiment, the immunogenic composition enables the elicitation of a memory immune response in said host or subject. According to a particular aspect, the protection conferred can be appreciated by measuring the level of virus (viral load) after a subsequent challenge/infection of a host, as shown in the Examples, and observing the level of viruses remaining in said host, which is preferably kept at a low level (according to the guidance provided in the experimental section, or observing virus clearance (complete/sterilizing protection). Alternatively, or in addition, the protection conferred can be appreciated by measuring the humoral response, i.e., the level of antibodies raised in the host. A protective immune response is one that reduces the risk that a subject will become infected with an arenavirus, especially a New World arenavirus as disclosed herein, and/or reduces the severity of an infection (including the spreading of the infection in an individual or the onset of the disease resulting from the infection as disclosed herein) with an arenavirus. Accordingly, protective immune responses include responses of varying degrees of protection.

In some embodiments, administering the active ingredients defined herein, according to any embodiment, reduces the risk that a subject will develop an infection with a New World arenavirus, especially selected from the group of a Machupo virus (MACV), a Sabia virus (SABV), a Chapare virus (CHAPV), a Junin virus (JUNV) and a Guanarito virus (GTOV), by at least about 10%, at least about 20%, at least about 25%, at least about 30%, at least about 35%, at least about 40%, at least about 50%, at least about 60%, at least about 70%, at least about 80%, at least about 90%, or more, compared to the risk of developing an infection with said arenavirus(es) in the absence of administering the said active ingredients.

In some embodiments, administering the active ingredients defined herein, according to any embodiment, reduces the symptoms of an infection or the symptoms of the disease related to the infection as disclosed herein in the subject with a New World arenavirus, especially selected from the group of a Machupo virus (MACV), a Sabia virus (SABV), a Chapare virus (CHAPV), a Junin virus (JUNV) and a Guanarito virus (GTOV), by at least about 10%, at least about 20%, at least about 25%, at least about 30%, at least about 35%, at least about 40%, at least about 50%, at least about 60%, at least about 70%, at least about 80%, at least about 90%, or more, compared to the manifestation of the symptoms of an infection with the said arenavirus(es) in the absence of administering the said active ingredients.

In the context of therapeutic uses, especially for immune response elicitation or vaccination purpose, described herein, it is to be understood that the active ingredients to be administered, can be administered in a single multivalent immunogenic composition or in admixture. According to particular embodiment for therapeutic use, especially for immune response elicitation or vaccination purpose, the active ingredients are administered as separate doses of active ingredients. Any active ingredient in this administration scheme can, however, be found within a separate “composition”: in that context a composition can contain at least two ingredients, which can be one or more active ingredient(s), i.e., the different recombinant live attenuated Mopeia viruses (MOPV) described herein—up to five in total, or more—and one or more of, if appropriate, an adjuvant, an immunostimulant component, a pharmaceutically acceptable carrier, a delivery vehicle, an excipient, a preservative, or any combination thereof, within their respective compositions, as described herein.

According to a particular embodiment, administration of the active ingredients or the multivalent immunogenic composition described in any embodiment disclosed in present description, to a human subject, enables the elicitation of a protective immune response that is a cellular (T cell-mediated immune response) and/or a humoral (antibody-mediated immune response) response against a New World arenavirus, in particular against one or more of New World arenavirus(es) selected from the group of a Machupo virus (MACV), a Sabia virus (SABV), a Chapare virus (CHAPV), a Junin virus (JUNV) and a Guanarito virus (GTOV).

For instance, the examples provided herein demonstrate that cellular protective immune response has been induced after immunization with MOPEVAC_{MAC} and challenge with MACV (see FIG. 12), through transcriptomic analysis of PBMCs.

The examples provided herein also demonstrate the elicitation of a humoral protective immune response (see FIG. 1C, 3B and 5A, B and C), through the level of neutralizing antibodies obtained. Of note, the intensity of the humoral response obtained is demonstrated by the fact that said humoral response was less intense with MOPEVAC_{LAS} and the obtained immunity was not sterilizing, whereas the obtained immunity is sterilizing with MOPEVAC_{MAC} and the pentavalent immunogenic composition described herein and in the Examples. This could not be expected since the vector scaffold in both cases, is the same. Furthermore, in the case of a multivalent immunogenic composition, a risk of bad cross-neutralization between closely related virus is generally present. For instance, deleterious cross-neutralization between closely related viruses, notably in the context of dengue viruses, has been reported. This phenomenon was therefore feared by the inventors of present invention. However, obtained data and present application demonstrate that such a deleterious effect of bad cross-neutralization was not present, and, in addition, that an increase of the neutralizing titer, which is very specific for the virus used for infection challenge, could be observed. This indicates that the immune memory is, surprisingly, reactivated in a very specific manner.

According to a particular embodiment, administration of the active ingredients or the multivalent immunogenic composition described in any embodiment disclosed in present description, to a human subject, enables the elicitation of an immune response that is a prophylactic immune response against a New World arenavirus infection, especially a New World arenavirus as described in any embodiment disclosed herein, or against a New World arenavirus disease. Examples of New World arenavirus infections, New World arenavirus infection symptoms and New World arenavirus diseases are given for instance in South American Hemorrhagic Fevers: A summary for clinicians deFrank, 2021, International journal of infectious diseases, https://doi.org/10.1016/j.ijid.2021.02.046 (12). New World arenavirus disease can include so-called Hemorrhagic Fevers, whose symptoms may include fever, malaise, headache, myalgia, arthralgia, oral enanthem, odynophagia, cough, nausea, vomiting, diarrhea, abdominal pain, gingival bleeding, dehydration, hemorrhagic symptoms, seizures, neurological symptoms (confusion, tremors, lethargy and coma), organ dysfunction, and/or bleeding, that can lead to death, although asymptomatic infections with New World arenavirus can arise.

According to a particular embodiment, administration of the active ingredients or the multivalent immunogenic composition described in any embodiment disclosed in present description, to a human subject, enables the elicitation neutralizing antibodies against a New World arenavirus, in particular selected among: a Machupo virus (MACV), a Sabia virus (SABV), a Chapare virus (CHAPV), a Junin virus (JUNV) and a Guanarito virus (GTOV) or several of said viruses, optionally against a New World arenavirus as described in any embodiment disclosed herein or part of present description.

A neutralizing antibody (NAb) is an antibody that defends a host from an infectious particle by neutralizing any biological effect it can have on the host. Neutralization renders the particle no longer infectious or pathogenic. Neutralizing antibodies are part of the humoral response of the adaptive immune system against viruses. Through specific binding to an antigen of said virus, neutralizing antibodies prevent the virus from interacting with its host cells it might infect and destroy. Immunity due to neutralizing antibodies may be sterilizing immunity, when the immune system eliminates the infectious particle before any infection takes place. The Example section of present application demonstrates that sterilizing immunity could be achieved.

According to a particular embodiment, administration of the active ingredients or the multivalent immunogenic composition described in any embodiment disclosed in present description, to a human subject, enables to achieve sterilizing immunity in the treated host, in particular after further challenge with a New World arenavirus, in particular selected among: a Machupo virus (MACV), a Sabia virus (SABV), a Chapare virus (CHAPV), a Junin virus (JUNV) and a Guanarito virus (GTOV) or several of said viruses, or any New World arenavirus as described in any embodiment disclosed in present description.

According to a particular embodiment, administration of the active ingredients or the multivalent immunogenic composition described in any embodiment disclosed in present description, to a human subject, achieves a cross-neutralization between any one of the New World arenavirus selected from the group of a Machupo virus (MACV), a Sabia virus (SABV), a Chapare virus (CHAPV), a Junin virus (JUNV) and a Guanarito virus (GTOV) or several of said viruses taken according to any possible combination thereof (including 2, 3, 4 of them), in particular achieves a cross-neutralization against another New World arenavirus, especially a pathogenic New World arenavirus.

Indeed, as shown in the experimental section herein, a cross-neutralization was observed (FIG. 2E) after vaccination with MOPEVAC_{MAC}. In addition, administration of MOPEVAC_{NEW} demonstrated that the protection achieved was free of deleterious effects (such as those due to a bad cross-neutralization, for example).

According to particular aspect, the protection conferred avoids any destructive cross-reactivity, i.e., the situation where immune response to one arenavirus can interfere with or lower the immune response to a different arenavirus, amongst the arenaviruses described herein. To the contrary, the experimental section demonstrates that, surprisingly and strikingly, no loss of efficacy arose when the effects of vaccination with MOPEVAC_{MAC} and MOPEVAC_{NEW} are compared.

The invention also concerns the active ingredients or immunogenic compositions described in any embodiment described herein, for use in an administration scheme in a human host in need thereof, where the active ingredients or immunogenic compositions described in any embodiment described herein are administered to a human individual in need thereof according to a prime immunization regimen or according to a prime-boost immunization regimen.

It is observed that the cellular response observed for the vaccine described herein, especially given the experimental data provided in present application, is advantageously consistent with a single administration regimen, which comes as an advantage. In this context, it is also surprising that a pertinent antibody-based response can be observed on the basis of such a single administration regimen.

Of note, surprisingly, the experimental results provided herein show that the cellular immune response obtained with MOPEVAC_{MAC} and MOPEVAC_{NEW} is induced by a mechanism that is different from the mechanism at stake with MOPEVAC_{LASV} vaccination, where cytotoxic cellular immune response was reported. However, the transcriptomic experiments reported herein clearly evidence a T-cell immune response.

According to a particular embodiment using a prime boost immunization regimen, said prime-boost immunization regimen may be in particular a homologous prime boost regimen wherein the active ingredient(s) or multivalent immunogenic composition of the prime administration and those of the boost administration is (are) the same. According to a particular embodiment, the dosage of the composition administered as a prime composition and the dosage of the composition administered as a boost composition, are different. According to a particular embodiment, the dosage of the composition administered as a prime composition and the dosage of the composition administered as a boost composition, are the same.

According to another particular embodiment using a prime boost immunization regimen, said prime-boost immunization regimen is implemented so that the active ingredient(s) or multivalent immunogenic composition of the prime administration and those of the boost administration is (are) different. According to a particular embodiment, the dosage of the composition administered as a prime composition and the dosage of the composition administered as a boost composition, are different. According to a particular embodiment, the dosage of the composition administered as a prime composition and the dosage of the composition administered as a boost composition, are the same.

A suitable dose of the active ingredient(s) of the invention, in a single and optionally separate composition, or associated in plurality within a composition, to be administered may be in the range of 1.10² and 1.10⁷ ffu (Focus-forming units), or between 1.10³ and 1.10⁶ ffu, or in any range where the boundaries are selected from 1.10², 1.10³, 1.10⁴, 1.10⁵ and 1.10⁶ for the lower range, and selected from 1.10³, 1.10⁴, 1.10⁵, 1.10⁶ and 1.10⁷ for the higher range, as measured by virus titration (see description of the unit, provided in the present description above).

According to a particular embodiment, the dose of the active ingredient(s) of the invention, in a single and optionally separate composition, or associated in plurality within a composition, to be administered, is as low as 1.10⁴, 1.10³ or 1.10² ffu.

The invention also relates to a combination of active ingredients comprising at least the five different recombinant live attenuated Mopeia viruses (MOPV) specifically described herein or in any part of present description, or a multivalent immunogenic composition comprising the said active ingredients as described in any embodiment or part of present description, for use in the treatment of a mammalian host, especially a human host, which has been infected with a New World arenavirus, in particular a New World arenavirus selected from the group of a Machupo virus (MACV), a Sabia virus (SABV), a Chapare virus (CHAPV), a Junin virus (JUNV) and a Guanarito virus (GTOV),
wherein the active ingredients are administered as a single composition or as separate active ingredients, especially in the same administration step, and
wherein optionally the active ingredients or multivalent immunogenic composition are, within their respective compositions, either free of or associated with adjuvant(s), especially adjuvant(s) of the immune response and/or immunostimulant component(s),
and optionally wherein the active ingredients or multivalent immunogenic composition are, within their respective compositions, associated with pharmaceutically acceptable carrier(s), delivery vehicle(s), excipient(s), preservative(s), or any combination thereof.

According to these embodiment(s), therapeutic treatment against New World arenaviruses infection(s), symptom(s) or disease(s) caused by New World arenaviruses infection(s) is sought. Reference is made to the description made above regarding symptom(s) and disease(s) referred to. Differently said, the invention also encompasses a method of inducing a therapeutic immune response against a New World arenavirus in a subject infected with a New World arenavirus. Such a method may comprise administering an effective amount of active ingredients as defined herein, such as in the form of an immunogenic composition as defined herein, to a subject infected with a New World arenavirus.

By “therapeutic treatment”, it is meant that administration results in improving the clinical condition of a subject who has been infected with a New World arenavirus as defined in any embodiment herein, who suffers from symptom(s) or disease(s) caused by New World arenaviruses infection(s) as defined herein (or may be asymptomatic). Such treatment aims at improving the clinical status of the infected subject, especially human subject, by diminishing the viral load caused by the infection(s) and/or eliminating or lowering or alleviating the symptoms associated with the condition(s) defined herein and/or in a particular embodiment, restoring to health.

According to a particular embodiment, it is meant by “treating the infection” or “therapeutic treatment”, the fact of abolishing, or preventing, or decreasing the mortality associated with the infection(s) by arenaviruses, especially New World arenaviruses, in particular selected from the group of a Machupo virus (MACV), a Sabia virus (SABV), a Chapare virus (CHAPV), a Junin virus (JUNV) and a Guanarito virus (GTOV), in an extent that the odds of survival to the infection(s) are increased. According to a particular embodiment, a treatment according to the invention comes with 40%, or 50%, or 60%, or 70% reduction of the mortality rate for the treated subject.

According to a particular embodiment, it is also meant by “treating the infection” or “therapeutic treatment”, protecting the subject from more severe consequences, on its health status, of the treated disease, compared to the consequences that would arise in the absence of treatment. This includes eliminating or lowering or alleviating the symptoms associated with the disease(s).

All features described herein, in any embodiment of present description, with respect to a combination of active ingredients comprising at least the five (especially five) different recombinant live attenuated Mopeia viruses (MOPV) discussed in present description, or a multivalent immunogenic composition or vaccine or therapeutically effective composition comprising the said active ingredients, also apply and can be relied upon in the context of therapeutic treatment, i.e., for example dosages, formulation, administration regimen, non-exhaustively.

The invention also relates method of preparing a recombinant live attenuated Mopeia virus (MOPV) in a eukaryotic host cell, said recombinant live attenuated Mopeia virus (MOPV) comprising an heterologous nucleic acid encoding a New World arenavirus GPC from an arenavirus selected among: Machupo virus (MACV), Sabia virus (SABV), Chapare virus (CHAPV), Junin virus (JUNV) and Guanarito virus (GTOV), wherein the method comprises the steps of:
transfecting the eukaryotic host cell with plasmids wherein:
   a first plasmid that comprises a polynucleotide which is an expression cassette encoding the L segment antigenomic transcript of a Mopeia vRNA (L vRNA segment expression cassette);
   a second plasmid that comprises a polynucleotide which is an expression cassette encoding a chimeric S segment antigenomic transcript of a Mopeia vRNA, in particular a S segment that is deleted for the ORF of the glycoprotein precursor (GPC) of the Mopeia virus, wherein the polynucleotide comprises (i) the ORF of the GPC protein of a New World arenavirus selected among: Machupo virus (MACV), Sabia virus (SABV), Chapare virus (CHAPV), Junin virus (JUNV) and Guanarito virus (GTOV), and (ii) the ORF of a nucleoprotein (NP) protein which is mutated by amino acid residue substitution(s) in the wild type NP of the Mopeia virus to have attenuated exonuclease activity;
   an expression cassette for the L protein of the Mopeia virus wherein said cassette is either present as an insert in the second plasmid or is contained in a third plasmid;
   an expression cassette for the NP protein of the Mopeia virus wherein said cassette is either as an insert in the first plasmid or is contained in a fourth plasmid;
allowing ribonucleoproteins of the recombinant Mopeia virus to form and expression of the New world arenavirus GPC gene to assemble into recombinant live attenuated viral particles; and,
recovering recombinant live attenuated Mopeia virus expressing the GPC of a New World arenavirus selected among: Machupo virus (MACV), Sabia virus (SABV), Chapare virus (CHAPV), Junin virus (JUNV) and Guanarito virus (GTOV).

Depending on the eukaryotic host cells used the formed recombinant live attenuated Mopeia virus may be recovered after budding from the cell membrane.

In some embodiments, the supernatant of the eukaryotic cells expressing the recombinant live attenuated Mopeia virus is used in an additional step of amplification by adding the supernatant to VeroE6 cells.

In a particular embodiment, the expression cassette for the NP protein of the Mopeia virus contained in a fourth plasmid, contains a non-mutated NP protein.

In another particular embodiment, the expression cassette for the NP protein of the Mopeia virus found either as an insert in the first plasmid or contained in a fourth plasmid, contains a NP protein which is mutated by amino acid residue substitution(s) in the wild type NP of the Mopeia virus to have attenuated exonuclease activity.

The invention also relates to a recombinant live attenuated Mopeia virus (MOPV) comprising a GPC protein of a New World arenavirus selected among: Machupo virus (MACV), Sabia virus (SABV), Chapare virus (CHAPV), Junin virus (JUNV) and Guanarito virus (GTOV), obtainable or obtained from the method described in the paragraph above. The invention also relates to a cell comprising such a recombinant live attenuated Mopeia virus (MOPV).

In order to rescue recombinant live attenuated MOPV as a vector carrying a polynucleotide expressing a glycoprotein precursor of the envelope protein (GPC) of a New World arenavirus, a reverse genetic system as disclosed in the art (notably a system paralleling the disclosure of section “B. Reverse Genetic System for MOPV” of WO2017/068190) is used. Accordingly, a eukaryotic cell is used as a helper cell to express the recombinant MOPV particles. Such eukaryotic cell is transformed, especially transfected, with a plurality of polynucleotides encompassing:
a first plasmid that comprises a polynucleotide which is an expression cassette encoding the L segment antigenomic transcript of a Mopeia vRNA (L VRNA segment expression cassette);
a second plasmid that comprises a polynucleotide which is an expression cassette encoding a chimeric S segment antigenomic transcript of a Mopeia vRNA, in particular a S segment that is deleted for the ORF of the glycoprotein precursor (GPC) of the Mopeia virus, wherein the polynucleotide comprises (i) the ORF of the GPC protein of a New World arenavirus selected among: Machupo virus (MACV), Sabia virus (SABV), Chapare virus (CHAPV), Junin virus (JUNV) and Guanarito virus (GTOV), and (ii) the ORF of a nucleoprotein (NP) protein which is mutated by amino acid residue substitution(s) in the wild type NP of the Mopeia virus to have attenuated exonuclease activity;
an expression cassette for the L protein of the Mopeia virus wherein said cassette is either present as an insert in the second plasmid or is contained in a third plasmid;
an expression cassette for the NP protein of the Mopeia virus wherein said cassette is either as an insert in the first plasmid or is contained in a fourth plasmid;

In a particular embodiment, the first, second, and when used, the third and fourth polynucleotides are DNA or cDNA.

In a particular embodiment, the expression cassette for the NP protein of the Mopeia virus contained in a fourth plasmid, contains a non-mutated NP protein, i.e., a wild type NP protein. Although not mandatory, this may favor the rescue. Of note, in this case the expression of the wild type NP protein is strictly limited to the said fourth plasmid.

In another particular embodiment, the expression cassette for the NP protein of the Mopeia virus found either as an insert in the first plasmid or contained in fourth plasmid, contains a NP protein which is mutated by amino acid residue substitution(s) in the wild type NP of the Mopeia virus to have attenuated exonuclease activity.

In some embodiments, for the production of the recombinant live attenuated MOPV according to the invention by reverse genetic system, the polynucleotides encoding the L and S segment of the genome of the MOPV strain AN21366 (GenBank accession numbers JN561684.1 and JN561685.1: SEQ ID NO: 2 and SEQ ID NO: 3) are used to derive the recombinant virus particles expressing a GPC characteristic of a New World arenavirus by reverse genetics.

In a particular embodiment, the MOPV strain encoding the antigenomic transcript for the L and the S segments to provide the first, and optionally the third and the fourth polynucleotides is the MOPV strain AN21366 (GenBank accession numbers JN561684.1 and JN561685.1: SEQ ID NO: 2 and SEQ ID NO: 3). In a particular embodiment, the second polynucleotide encodes a modified sequence of the S antigenomic transcript of the MOPV strain AN21366 and the modifications with respect to said transcript encompass or consist of:
(i) mutation of the sequence of the S segment encoding the nucleoprotein protein (NP) in order to express a nucleoprotein with impaired or abolished, especially with attenuated, exonuclease activity; in a particular embodiment the expressed mutated NP comprises the mutation of amino acid residues as disclosed herein; and,
(ii) deletion of the sequence of the S segment encoding the GPC protein of MOPV strain AN21366 and replacement by the sequence (ORF) encoding the GPC protein of the New World arenavirus as disclosed herein.

In some embodiments, the first, second, and when used, the third and/fourth polynucleotides are independently of each other mutated with respect to their functionally corresponding sequence in the MOPV strain from which they originate so that when aligned the polynucleotide used in the invention has at least 80%, at least 85%, at least 90%, at least 91%, at least 92%, at least 93%, at least 94%, at least 95%, at least 96%, at least 97%, at least 98%, at least 99% identity with the corresponding sequence of the considered MOPV, in particular with the corresponding sequence in the MOPV strain derived from Mopeia AN21366 strain (GenBank accession numbers JN561684.1 and JN561685.1: SEQ ID NO: 2 and SEQ ID NO: 3).

Software tools for carrying out identity percentage calculation are commonly known and readily accessible to the skilled person: they can in particular be freely accessible over the internet. The literature provides details regarding available tools. In particular, identity percentages can conventionally be calculated through local or global, sequence alignment algorithms and their available computerized implementations. In a particular embodiment, identity percentages are calculated over the entire length of the compared sequences. Global alignments, which attempt to align every residue in every sequence, are most useful when the sequences in the query set are similar and of roughly equal size. Computerized implementations of the algorithms used are generally associated with default parameters in the literature, which can be used for running on or the other of such algorithm(s). The skilled person can readily adapt the same taking into account its objective or the sequences comparison made.

In an embodiment, the first and/or the second and/or when used herein the third polynucleotides and/or fourth polynucleotide comprise respectively the sequence of SEQ ID NO: 14 (L Segment) cloned in the plasmid pRF108 for the first polynucleotide, the modified sequence of any of SEQ ID NO: 15, 16, 17, 18 and 19 for the second polynucleotide for the Guanarito, the Carvallo, the Chapare, the Sabia and the Junin viruses, respectively (S Segments, which differ depending upon the virus), the sequence of SEQ ID NO: 20 corresponding to the sequence LpoI Mopeia for the third polynucleotide (in particular cloned in the plasmid pTM1), the sequence of SEQ ID NO: 21 corresponding to the sequence NP Mopeia for the fourth polynucleotide (in particular cloned in the plasmid pTM1). SEQ ID NO: 14 to 21 are the sequences of the polynucleotides that have been used in the experiments reported in the Experimental Section. Annotated versions of theses sequences are provided herein (see below).

For the purpose of preparing rescued recombinant live attenuated Mopeia virus, the first, second, and when used herein the third and fourth polynucleotides comprise transcription and expression control of sequences such as promoter and terminator sequences. When the NP expression cassette and the L protein expression cassette are contained in the respectively first and second polynucleotides their expression is driven by expression control sequences that are different from the expression control sequence contained in the L vRNA segment expression cassette respectively in the S vRNA segment expression cassette.

In an embodiment, the complete transcription of viral segments to provide the sequences of the first and second polynucleotides comprising respectively the L and S sequences of the MOPV in antigenomic orientation, is obtained starting from viral RNA extracts and the obtained cDNA is cloned into a plasmid that drives the correct transcription under the control of the murine RNA polymerase I. For both correct transcription and replication of the viral segments, an extra non templated-G base, may be included at the beginning of the cloned sequences (Carnec, X. et al. Lassa virus nucleoprotein mutants generated by reverse genetics induce a robust type I interferon response in human dendritic cells and macrophages. J Virol 85, 12093-7 (2011)).

In an embodiment, each of the first, the second, and when used the third and fourth polynucleotides is provided on a plasmid suitable for transfection of the eukaryotic cell. All plasmids may be sequenced and where necessary corrected by site directed mutagenesis to match the consensus sequence of the wild type MOPV strain such as the AN21366 MOPV strain, except for purposely-introduced mutations to discriminate into the sequence encoding the NP of MOPV.

In an embodiment, a plasmid is used for generation of S and L antigenomic transcripts that start with a nontemplated G required for efficient transcription and replication of arenaviruses. The murine Poll promoter and terminator may be used to control the transcription of the of S and L antigenomic transcripts. pRF108 plasmids (Flick R., Pettersson R. F. 2001. Reverse genetics system for Uukuniemi virus (Bunyaviridae): RNA polymerase I-catalyzed expression of chimeric viral RNAs. J. Virol. 75:43-1655) containing the murine Pol I promoter and terminator (pPolI) may be used to express the transcripts of the S and L segments of the recombinant MOPV.

In a particular embodiment, the third and the fourth polynucleotides are cloned each on a pTM1 plasmid (Elroy-Stein O., Fuerst T. R., Moss B. 1989. Cap-independent translation of mRNA conferred by encephalomyocarditis virus 5′ sequence improves the performance of the vaccinia virus/bacteriophage T7 hybrid expression system. Proc. Natl. Acad. Sci. U.S.A. 86:26-6130), similarly to what is disclosed in Carnec et al. 2018 J Virol. 2018 May 29; 92(12): PMID: 29593043, describing the reverse genetics of the Mopeia virus. In Carnec et al., 2018, the expression cassette is identical to the expression cassette used in the experimentations reported herein, i.e., and expression cassette with a T7-IRESemcv promoter and a MCS-T7 terminator.

In order to enable their transcription and expression in the recombinant eukaryotic cell, the third and fourth polynucleotides comprise transcription regulatory sequences suitable to enable expression of the polypeptides that they respectively encode. In a particular embodiment, the transcription regulatory sequences for the third and fourth polynucleotide comprise a T7 promoter and terminator.

In a particular embodiment, the eukaryotic cells used for the rescue of the recombinant live attenuated Mopeia virus express the T7 RNA polymerase. The third and fourth plasmids require expression of T7 RNA polymerase to transcribe the NP and LpoI genes coded by the sequences contained in these plasmids.

In another particular embodiment, the eukaryotic cells transformed with the first, second, and optionally third and optionally fourth polynucleotides are further capable of expressing a RNA polymerase such as the T7 RNA polymerase. In particular, the eukaryotic cells used for the rescue of the recombinant live attenuated Mopeia virus constitutively express the T7 RNA polymerase of T7 bacteriophage.

The preparation of plasmids for the rescue of the recombinant live attenuated MOPV is also disclosed and illustrated in WO2017/068190, in particular in the Material and Methods of the Examples and in Example 1 of WO2017/068190. The strategy used to prepare the plasmid derived from the S segment of MOPV with the GPC gene of the JUNV virus is also identical to the strategy disclosed in WO2017/068190.

In some embodiments, the eukaryotic cells of interest for rescuing the recombinant live attenuated MOPV are BHKT7/9 cells (cell line that expresses the T7 RNA polymerase). These cells are maintained in culture as described in Carnec, X. et al. Lassa virus nucleoprotein mutants generated by reverse genetics induce a robust type I interferon response in human dendritic cells and macrophages. J Virol 85, 12093-7 (2011).

In some embodiments, the plasmids encoding the first, second, third and fourth polynucleotides are transfected in the host eukaryotic cells with a 1:1:1:1 ratio.

In a particular embodiment of the process to rescue recombinant live attenuated MOPV encoding the GPC of a New World arenavirus, an amplification step is carried out after production of the virus in the host cells. Vero cells may be used to enable amplification and recovery of virus stock. In particular, Vero cells are grown in Glutamax Dulbecco Modified Eagle's Medium (DMEM—Life Technologies) supplemented with 5% FCS and 0.5% Penicillin-Streptomycin. The invention also relates to a method of preparing recombinant live attenuated Mopeia virus in a eukaryotic host cell, as further described hereafter.

The invention also relates to a method of preparing a multivalent, in particular pentavalent, immunogenic composition comprising recombinant live attenuated Mopeia viruses (MOPV) expressing a GPC protein of a New World arenavirus selected among: Machupo virus (MACV), Sabia virus (SABV), Chapare virus (CHAPV), Junin virus (JUNV) and Guanarito virus (GTOV), the method comprising the steps of:
a. Preparing recombinant live attenuated Mopeia viruses (MOPV) according to the method described according to any embodiment disclosed herein; and,
b. Associating the recovered recombinant live attenuated Mopeia viruses wherein each of the recombinant MOPV expresses a GPC of a New World arenavirus selected from the group of Machupo virus (MACV), Sabia virus (SABV), Chapare virus (CHAPV), Junin virus (JUNV) and Guanarito virus (GTOV) to provide a multivalent immunogenic composition or vaccine wherein collectively all of said GPC are expressed and wherein the quantitative proportion of each valence of MOPV in the composition is identical.

According to a particular embodiment, such a method of preparing a multivalent, in particular pentavalent, immunogenic composition provides a multivalent immunogenic composition having any one of the features independently described in any section of present description, or combinations of such sections.

The invention also relates to a method for inducing an immunogenic response (e.g., prophylactic or therapeutic immunogenic response) against a New World arenavirus in a subject, especially a New World arenavirus as defined herein, comprising administering to said subject the active ingredient(s) or multivalent immunogenic composition(s) as defined in any embodiment herein, including combinations of features from different embodiments. Optionally, an active ingredient or a multivalent immunogenic composition, especially when formulated for use as a vaccine or a therapeutic, can be administered in combination with an adjuvant or an immunostimulant component, wherein the adjuvant or immunostimulant component is administered before, concomitantly with, or after administration of the active ingredient(s) or multivalent immunogenic composition. Optionally, any one of: pharmaceutically acceptable carrier(s), delivery vehicle(s), excipient(s), preservative(s), or any combination thereof can be present, based on the same description for this feature as the description provided throughout present description.

Of note, instant description makes use of the “for use” wording for defining therapeutic, especially selected among: immunogenic, prophylactic, vaccine and therapeutic (i.e., treatment of a mammal host, especially human host infected with a New World arenavirus) applications. Throughout the description, a wording using the expression “method of” can be alternatively be used without the intended meaning being different.

The invention also relates to the use of an active ingredient or multivalent immunogenic composition, or a plurality of active ingredients or multivalent immunogenic composition(s) according to any one of the embodiments or possible combinations described herein, for the preparation (or manufacture) of a medicament having the immunogenic, prophylactic, or vaccine effect(s) described herein. Reference is made to any one of the embodiments described herein, and all combinations thereof, with respect to purpose(s) of said medicament/vaccine.

Other examples and features of the invention will be apparent when reading the examples and the Figures, which illustrate the experiments conducted by the inventors, in complement to the features and definitions given in the present description.

### LEGEND OF THE FIGURES

FIG. 1 Immunization of cynomolgus monkeys with MOPEVAC_{MACV} and antibody response. a, Schematic view of the experiment. Long bars represent each week. The days of vaccination, sampling, challenge or necropsy are indicated with colored arrows. b, Bi-segmented organization of MOPEVAC_{MACV} genome. The S segment is mutated in the exonucleasic region of the nucleoprotein (NP) and the glycoprotein precursor (GPc) of MACV replaces the GPc of Mopeia virus (MOPV). The L segment is the one of wild type MOPV (L: polymerase, Z: zinc finger matrix protein, UTR: untranslated regions). c, Antibody response during the immunization period. IgG titer is the last dilution of plasma sample that is still positive in ELISA experiment. Each dot represents the mean of the group (n=4) and the error bar indicate the standard error. Neutralization titer corresponds to the last dilution of plasma that still neutralize more of 50% of the dose of Machupo virus (MACV) used. Each dot represents an individual value.

FIG. 2 Post challenge monitoring of vaccinated and control monkeys and antibody response. Unvaccinated controls are represented in red, animals vaccinated with a single injection are in dark blue and animals vaccinated twice are in light blue. The lines connect the dots for each individual. a, At each sampling time, the clinical score and the body weight were evaluated. We then calculated the weight loss, considering the baseline at the day of challenge. b, Plasma was used to measure biochemical parameters. ALT: alanine aminotransferase, AST: Aspartate aminotransferase. c, Viral RNA was quantified by RT-qPCR at each sampling time. We performed the quantification of infectious viruses on positive samples. Negative values are represented at the threshold of detection. d, IgG and neutralization titers were assayed. The representation is like in FIG. 1c. e, To evaluate cross-neutralization samples from the immunization period were assayed using MOPEVAC viruses that carry the different GPc genes. The arrow indicates the date of the boost injection.

FIG. 3 MOPEVAC_{NEW} experiment to evaluate its ability to protect against all pathogenic New World (NW) arenaviruses. a, Schematic representation of the protocol. Monkeys were vaccinated or not with a prime boost strategy (n=6) and then infected with either MACV or GTOV (four groups n=3). The representation is like in FIG. 1a. b, Apparition of neutralizing antibodies was measured during the immunization period and evaluated for each pathogenic NW arenavirus. The representation is like in FIG. 2e. c, IgG titers were calculated from ELISA assay on plasma samples during the immunization period. The arrow indicates the second injection of the vaccine.

FIG. 4 Challenge of vaccinated or not NHP with GTOV and MACV. Unvaccinated NHP were challenged with GTOV (gray, n=3) or MACV (red, n=3). Vaccinated NHP received the same challenge with GTOV (orange, n=3) or MACV (blue, n=3) a, The clinical score and the loss of weight were evaluated at each sampling time like in FIG. 2a. b, Viral RNA was quantified by RT-qPCR at each sampling time. We performed the quantification of infectious viruses on positive samples. Negative values are represented at the threshold of detection.

FIG. 5 Antibody response after challenge. The colors used as the same as in FIG. 4. a, IgG titers were evaluated by ELISA and represent the last dilution of plasma that is still positive. The dots indicate the mean of the group and the error bar the calculated standard error (available if n=3). Vaccinated NHP are n=3 during the whole experiment, unvaccinated GTOV are n=3 until day 12 and n=2 from day 16 to day 29 and unvaccinated MACV are n=3 until day 12, n=1 for the other dots. b, Neutralization titers were defined against wild type viruses at days 0, 12 and the date of necropsy. The experiment was performed like for FIG. 1c. c, MOPEVAC viruses were used to evaluate the neutralization titers. The experiment was performed with the same protocol.

FIG. 6 Recording of the body temperature after challenge. Recording systems were implanted in the NHP to evaluate the body temperature all along the protocol. Some were defective, we thus obtained data for seven NHP: the three controls, three prime only vaccinated animals and one prime boost. For some animals, the record was stopped unintentionally for a small period for 5 animals, this is clearly visible in the graphs.

FIG. 7 Hematological parameters and viral loads in the organs at the day of necropsy. a, Cell counts and hemoglobin concentrations were measured at each sampling time on whole blood using a hematological analyzer. b, Viral RNA was quantified by RT-qPCR from crushed organs or cells. The positive samples were evaluated for infectious virus titers.

FIG. 8 Body temperature before and after challenge in MOPEVAC_{NEW} experiment. Intraperitoneal implants recorded the body temperature all along the experiment. One point every 15 min was used in the graphs. a, Post immunization period in vaccinated NHP. They all received the same vaccine but the color indicate the virus further used for challenge. b, Vaccinated and control animals body temperature after challenge.

FIG. 9 Viral loads in organs and immune-preserved compartments. a, Viral RNA was quantified by RT-qPCR from crushed organs or cells. The positive samples were evaluated for infectious virus titers. b, Viral RNA was quantified from cerebrospinal fluid (CSF) and eye vitreous humor and infectious virus titration was also performed.

FIG. 10 Hematological and biochemical parameters after challenge in the MOPEVAC_{NEW} experiment. a, Cell counts and hemoglobin concentrations were performed at each sampling time after challenge using a hematological analyzer. b, Biochemical parameters were assayed on heparin-lithium plasmas at each sampling time using a veterinarian analyzer. C-reactive protein (CRP), alanine aminotransferase (ALT), aspartate aminotransferase (AST) and plasmatic albumin are represented here.

FIG. 11A: Transcriptomic analysis performed with PBMC from MOPVAC_{LASV} immunized animals. The genes associated with the innate immune response are represented. B: The same representation than in A for MOPVAC_{MACV} vaccinated animals. C: IFNγ producing T cells were measured after stimulation of whole blood with LASV overlapping peptides of the NP or GPC protein. The mean titers of IgG specific antibodies against LASV and neutralizing antibodies are indicated.

FIG. 12 Immune responses induced after immunization with MOPEVAC_{MAC} and challenge with MACV. a, Transcriptomic analysis of PBMCs. For each pathway, the changes in gene expression after immunization are indicated by a color that represents the mean of scaled normalized counts (n=4). Box plots represent the evolution of the mean of the pathway and stars indicate the significance of the difference from day 0 (***p<0.001, **p<0.01, *p<0.05). b, GPC- and NP-specific responses of CD4 and CD8 T cells after whole blood stimulation was evaluated by measuring IFNγ synthesis. The difference from the non-stimulated condition is represented and a bar indicates the standard error (Vacc: n=4, Ctrl: n=3). c, Transcriptomic analysis of PBMCs obtained after challenge. Data were analyzed and are represented as in a (n=3).

FIG. 13 a, Homogeneity of replication capacities and virus stability. Vero E6 cells were infected at moi=0.0001 and cell supernatants were collected every day until day 7 and titrated (left graph). Viral titers of the different MOPEVAC at each passage from passage two to ten (right graph). The mean of two experiments is represented for both graphs. b, Sequence stability after multiple passages. Evolution of vaccine candidate genomes during serial passages in VeroE6 cells is shown. The consensus genome sequences of the five vaccine candidates were determined at passages 2, 5, and 10 and compared to the initial sequences (P2). The changes in codon sequences were indicated as well as the position of the mutation in the genome. The amino acid changes are indicated for non-synonymous mutations whereas synonymous mutations are colored in green. The passage after which the mutation has been detected is indicated by the presence of a colored box. c, Detection of IgG directed against all viruses included in the MOPEVAC vaccine. IgG in plasma samples were tested on 293T cells expressing GPC from NWAs, MOPV and control cells without GPC expression. The percentage of cells that were recognized by IgG from plasma samples at day 82 post-immunization is represented for each animal. All animal indicated Vacc received the same MOPEVAC vaccine and all Ctrl animal received only the excipient. The indication MACV ou GTOV indicates only the virus used for challenge after the immunization period.

### MATERIAL AND METHODS

### Studies Design

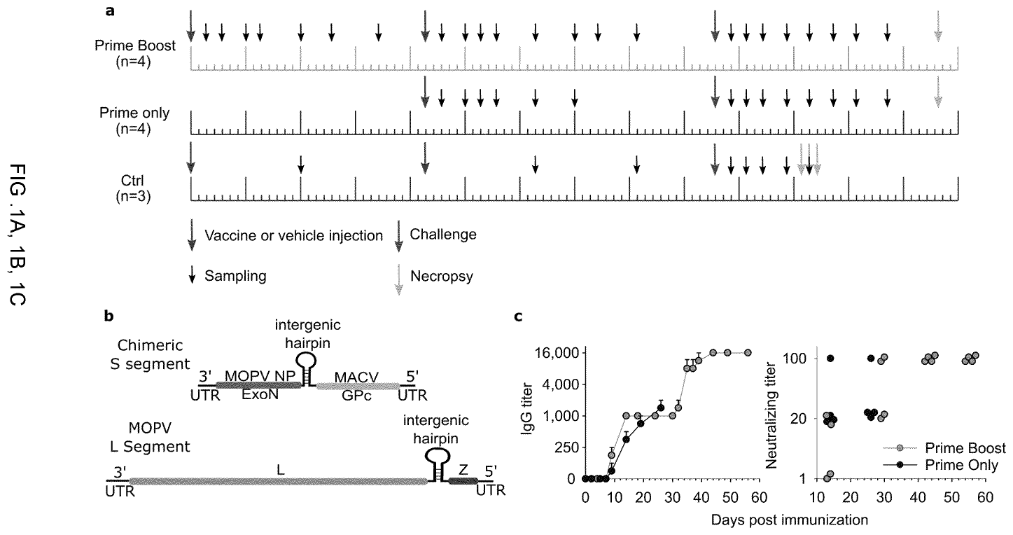

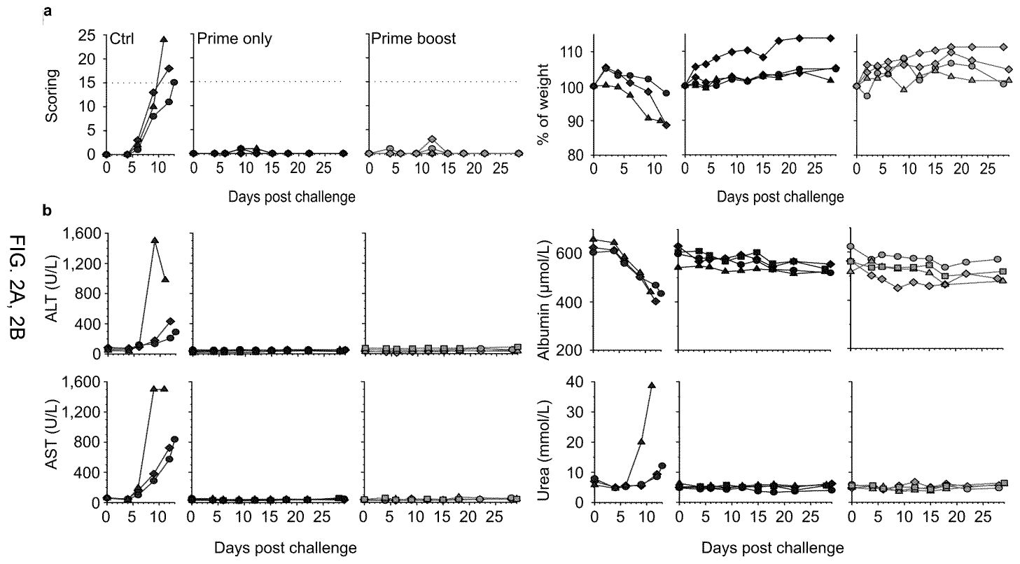

We assayed the efficiency of MOPEVAC_{MACV} vaccine on eleven cynomolgus monkeys (Macaca fascicularis) aged of 2.5 years and that weighted 2.3 to 3.9 kg. They were previously implanted with intraperitoneal systems that allowed to record and visualize in real time the body temperature. Unfortunately, most of them were deficient and we did not obtain the data during the whole immunization period. The protocol was first performed in a BSL2 facility (SILABE, France). Three animals were injected with vehicle and were unvaccinated controls, and two groups of four animals were vaccinated once or twice respectively. Vaccine or vehicle injection were performed intramuscularly at days 0 and 30 using 2.6×10⁶ ffu/ml of MOPEVAC_{MACV}. The group that received a single dose of vaccine was immunized at day 30. Blood samples, urine, oral and nasal swabs were taken periodically (FIG. 1A). The immunization protocol was approved by the ethical committee “Comité Régional d'Ethique en Matière d'Expérimentation Animale de Strasbourg” and registered with the number APAFIS #18970-2019020616112503 v8 (2019 Jul. 23).

Animals were reimplanted at the end of the immunization period using a new subcutaneous system to record the body temperature, this device did not offer the possibility to visualize the data in real time. The animals experienced some difficulties in the cicatrisation process and some of them lost the implant throught the scar and some had to be retired to fight against bacterial infection. The animals were moved to the BSL4 laboratory (P4 Jean Merieux-INSERM, Lyon, France) at day 56 post immunization. After ten days of acclimation they were all infected subcutaneously with 3 000 ffu of MACV (strain Caravallo). Samples were taken every two days until day 6, every three days from day 6 to day 18, and on day 22. The end of protocol was planned at days 28-29. Finally, we could obtain the full record of body temperature for 7 animals (3 controls, 3 of the group vaccinated with one shot and 1 for the two shots group).

Each day, the animals were evaluated and a clinical score was calculated based on behavior, body temperature, dehydration, loss of weight, clinical signs and reactivity. A clinical score of 15 was defined as a limit in the protocol and the animal was euthanized. The BSL4 procedure was approved by the Comité d'éthique pour l'expérimentation animale en neurosciences (Lyon, France) and registered with the number APAFIS #18397_2019011010351235_v4 (2019/03/15).

The second experiment was conducted in the same labs and with equivalent protocols and procedures.

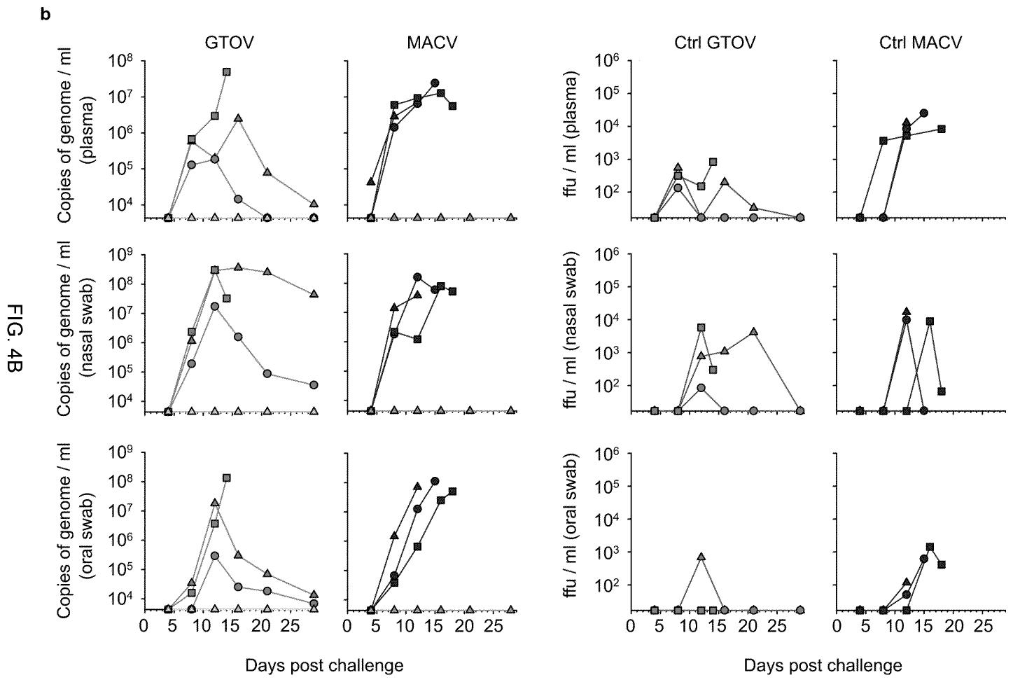

This time, the body temperature was efficiently recorded during the whole procedure using intraperitoneal loggers. Twelve cynomolgus monkeys were included, aged of three years and that weight 2.5 to 3.4 kg. Six received the vehicle and six were vaccinated with MOPEVAC_{NEW}. The immunization was performed at days 0 ant 56. The animals were moved to the BSL4 laboratory at day 89. After a period of acclimation of ten days, they were challenged with 4500 ffu of MACV or 3000 ffu of GTOV. Each virus was inoculated two six animals: three vaccinated and three unvaccinated (FIG. 4A). The protocol was approved by the same ethical committees, APAFIS #18970-2019020616112503 v8 (2019 Jul. 23) for the immunization protocol and APAFIS #28798_2020122311384240_v2 (2021 Feb. 11) for the BSL4 procedure. During the protocol, we observed important and unexpected weight loss for some animals. The score calculation did not provide additional points for weight loss beyond 10%. The veterinary decided that more than 20% of weight loss should be an ethical endpoint.

### Viruses

The MOPEVAC platform previously described ¹ consists in a Mopeia virus that carries the GPC of the virus of interest in place of its own GPC and that is mutated in NP gene to abolish the exonucleasic function. The attenuated virus obtained was produced on VeroE6 cells and in DMEM 2% FCS. MOPEVAC_{MACV} was then concentrated by centrifugation on filter tubes with a 1 000 kDa cutoff. A vehicle solution was prepared with uninfected VeroE6 supernatant in the same conditions and was used in the control animals in place of the vaccine injection.

MOPEVAC_{NEW} is a mix of equivalent quantities of infectious particles of MOPEVAC_{MACV, GTOV, SABV, JUNV, CHAPV,} i.e. is a pentavalent composition wherein each recombinant. It was produced in the same conditions except for the concentration method. The cell supernatant was precipitated with PEG solution (Abcam). After an overnight incubation at +4° C. with gentle agitation, this was centrifuged for 3 h at 4696 g and the pellet was resuspended in DMEM 2% FCS.

MACV, strain Carvallo (GenBank accession number AY619643—SEQ ID NO: 9), GTOV, strain INH95551 (GenBank accession number AY129247-SEQ ID NO: 13) and JUNV, strain P2045 (GenBank accession number DQ854733-SEQ ID NO: 12) were produced on VeroE6 cells in DMEM 2% FCS. The clarified cell supernatant was diluted in PBS for the inoculation of the virus in the animals. The same viruses were used for further experiments on biological samples from the experiments. The CHAV strain used is the CHAV strain 810419 (GenBank accession number NC_010562—SEQ ID NO: 11) and the SABV strain used is the SABV strain SPH114202 (GenBank accession number NC_006317-SEQ ID NO: 10).

### ELISA

Viral specific IgG detection was performed on plasma samples. Briefly, antigens were coated on a polysorp 96 microwells plate, diluted 1/500 or 1/1 000 in PBS. After an overnight incubation at +4° C., they were blocked for 1 h with PBS 2.5% BSA. Plasma samples were added to the wells for 1 h at 37° C. diluted from 1/250 to 1/16 000 in PBS, 2.5% BSA and 0.5% Tween 20 before a final incubation with anti-monkey HRP (Sigma). The attachment of the conjugated antibody was revealed with TMB and stopped with orthophosphoric acid. Between each step, plates were washed three times with PBS 0.5% Tween 20. Optical density was finally measured. Antibody titers correspond to the last dilution that is still positive.

### Quantitative RNA Analysis

RNA was prepared from liquid samples using QIamp viral RNA mini kit (Qiagen), or from cells or tissues using RNeasy mini kit (QIagen). Quantitative RT-qPCR was performed using SensiFAST Probe No-ROX One-Step kit (Bioline) on a LightCycler480 device (Roche). A standard RNA was used for quantification and we were able to detect 4 copies/μl of RNA. We performed a test sensitivity experiment using different matrix. We obtained a limit of quantifiable material of 6 ffu/ml in plasma and oral/nasal swabs for GTOV, 25 ffu/ml and 625 ffu/ml in plasma and swabs respectively for MACV. However, we were able to detect the material until 5 ffu/ml in plasma and 125 ffu/ml in swabs for MACV samples.

### Virus Titration

The samples that were positive in RT-qPCR were evaluated for the presence of infectious particles. For the organs, a piece was diluted in DMEM 2% at 10 mg/100 μl and put in a tube with three metal beads. It was crushed for 10 min à 30 beats per second. The solution obtained was centrifuged for 3 min at 1500 rpm to pellet the debris. The supernatant was used as the liquid samples for titration.

Samples were serially diluted in DMEM 2% FCS and incubated on VeroE6 cells plates for 1 h at 37° C. A medium supplemented with carboxymethylcellulose was added and the plates were incubated for 1 week.

Cells were then fixed for 20 min with 4% formaldehyde. For focus forming units count, plates were permeabilized for 5 min with 0.5% triton, stained for 1 h with anti-viral antibody and for one more hour with HRP conjugated secondary antibody. The reaction was finally revealed with NBT/BCIP (Thermo Scientific). Focus forming units were counted. For plaque forming units count, cells were colored with cristal violet solution (Sigma-Aldrich) diluted ½ in PBS for 10 min and washed with water. Plaques were counted.

MACV and JUNV were revealed using anti-Z MACV, MOPEVAC was stained with anti-Z MOPV. These antibodies were produced in order from rabbit (Agrobio). For SABV virus, we used an anti-monkey MACV obtained from the USAMRIID. The secondary antibodies were all coupled with HRP (Sigma). We did not get reactive antibodies for GTOV but the virus was lytic and we used cristal violet to reveal cell lysis. The threshold of detection was 17 ffu/ml for liquid samples and 0.5 ffu/mg for organs

### Virus Titration—Viral Titer Calculation

Day −1: Plate Vero E6 cells in 12-wells plates, 2, 5.10⁵ cells per well in DMEM 5% FCS. Incubate overnight at 37° C., 5% CO₂.

Day 0: Samples to be titrated are serially diluted in DMEM 2% FCS, currently using a dilution range from 10⁻¹ to 10⁻⁶.

Medium is removed from the cells and 300 μl per well of viral dilution is added to the cells for 1 h at 37° C., 5% CO₂.

After 1 h incubation at 37° C., 5% CO₂, DMEM 5% SVF diluted ½ in 2% carboxymethylcellulose solution is added to the cells, 1 ml per well.

The plates are incubated for 7 days at 37° C., 5% CO₂.

Day 7: Medium is carefully removed from the wells and cells are fixed using 4% formaldehyde in PBS for 20 min at room temperature.

Cells are washed two times in PBS and permeabilized in 0.5% triton X-100 in PBS for 5 min.

Cells are washed three times before staining with an antibody anti-Z protein of Mopeia virus from rabbit for 1 h at 37° C.

Cells are washed three times before adding an antibody anti-rabbit coupled with phosphatase alkaline.

After 1 h of incubation at 37° C., cells are washed three times and NBT/BCIP, a substrate of phosphatase alkaline, is added to the wells (350 μL/well). Wells are rinsed with water to stop the reaction.

The viral titer is calculated by counting the dots in the adequate dilution of sample. Each dot corresponding to an infectious particle in the sample.

### Seroneutralisation

Seroneutralisation experiments were conducted with wild type viruses or MOPEVAC viruses as explained in each relevant figure. Plasma samples were serially diluted in cell culture medium and a single viral dilution was added in the wells. After 1h incubation (37° C./5% CO₂) the mix of plasma and virus was added on cells. The infection was performed for 1h and media supplemented with carboxymethylcellulose was added. The cells were incubated for 1 week before immunostaining of infected cells or cristal violet coloration (cf virus titration). The neutralizing titer was the last dilution that allowed more than 50% of reduction of viral plaques in comparison with a condition without plasma.

### Hematological and Biochemical Analyses

Hematological parameters were analyzed on a MS9-5s (Melet Schloesing Laboratories) and biochemical analyses were performed on plasma from heparin lithium blood tubes using a Pentra C200 analyzer (Horiba).

### Polynucleotides Used

### First Polynucleotide: MOPEIA L Segment 7272 pb in Antigenomic Orientation—Accession Number JN561685—SED ID NO: 14

5′NC region: 1-57

RNA dependant RNA polymerase “Lpolymerase” ORF: 58-6771

Intergenic Region (IGR): 6772-6881

Z matrix protein ORF: 6882-7193

3′ NC region: 7194-7272

GCGCACCGAGGATCCTAGGCATTGTGACTCTCTCTTCTGAGGAACAAGTGTGTGGTGATGGAGGAGTTGCTGTCCGAGAGCAAAGA
TCTTGTGAGCAGGTACCTCTTAGAGGATGAGAGGCTTTCAAAACAGAAGCTAGCCTTCCTAGTTCAGACAGAGCCGAGAATGCTAT
TAATTGAGGGCCTTAAGTTGTTGTCACTCTGTATTGAGATAGACAGTTGCAAAGCCAATGGTTGTGAGCACAACTCAGAAGACCTT
TCTGTTGAAATCCTTCTGCAGAGACAAGGTGTTCTGTGCCCTGGTCTACCATTTGTGGTGCCAGATGGATTCAAATTCAGTGGGAA
CACATTAATATTACTGGAATGCTTTGTCAGGACTTCACCAATAAACTTTGAGCAAAAGTATAAAGAAGATACCATTAAGCTAGAGT
CTTTAAAGCCTGACTTATCATCTGTTGACATCATTCTGTTGCCCTTGATAGACGGCAGAACCAATTTCTACACTGATTTGTTTCCA
GAATGGGCCAATGAGAGGTTTAGGCATATCCTATTTAGCTTACTTGAATTCTCGCAGCAGTCATCAAAAATGTTTGAAGAATCTGA
GTACTCAAGGCTTTGTGAATCTTTAACAAAAGCAGGAGTGAGAACATCGGGCATTGAGAGCCTCAATGTTTTGACAGATTCTCGAT
CTGATCATTATGAGAGGGTGTTGGAGCTCTGCCACAGAGGCATCAATAACAAAATGTCCATTCTTGATGTGAAAAAAGAGATTGTA
TCAGAATTTCATGCTTTTAGGAATAAGCTTAAAGAGGGTGAGATAGAAAGACAATTTGTCAGGACAGATAGGCGACAGCTCTTGAG
AGATTTCAACAACCTTTATATTGACAGAGAGGGGGACACACCCTCAGAGATTGATCCTCTAAAAGAGAGGTTTGTAAAATCCTCGC
CTATGGTAACAGCGCTCTATGGTGACTACGACCGTTATAGGCAAGAGGGAGTTGATCGAGACAGCTGCTTGCAGAATCACTTCCAA
AGCTCTGTGCCTGGATGGAAGTCACTGTTGAATAAAATAAAATCACTAAAGTTATTGAACACCAGAAGAAAACTAATGCTGACTTT
TGACGCAATCATCCTTTTGGCCCACTTAAAGGATCTTAAATGTCACGGCGAGCTGTTAGGATCAGAATGGCTTGGCTCCTCATTCT
TAAGTGTGAACGACAGATTGGTGTCACTACAAGAAACACAAAAGGACCTTAAAAAGTGGATTGAAAGGAGAATGGTGAGTGCAATG
AAGAAGAAGGGAGGTGTAGGAACTCTGTGTCAGAGATCTGAGCTTATATTCTTTGACATCATAAACAAACTCCTCACAAAGGCCAA
AGAGGCATTATCCTCTGCCAGTTTGTGCTTTAGAGATTATGTTAAAGAGGAAGATATACTGGAGGAGGACAGTTACGAGAGGCTTA
TGTTAATGGAGAAAAGAGGGATTCAGCCAACAATGAGCTATGAGAAAGAAGAGGGGAATCAATTCCCCTACCCTCTTATTGAGTTG
GAAGCTGATTCCATAGAAGACCTGAGAAGACTATCTAGCATCTCTTTGGCATTGGTGAATTCAATGAAGACATCATCAGTAGCCAA
AGTGAGGCAAAATGAGTATGGTGCTGCAAGGTACAAACGTGTACGTTGTAAGGAAGCTTTTAATCAAAGCTTTATCATGGGAAGCG
GGAATTTCAACTTAATTTATCAGAAAACAGGAGAGTGCTCAAAATGTTATGCCATTAACAATCCTGAGAAGGGGGAGATTTGTTCA
TTCTATGCAGATCCAAAAAGGTTTTTTCCTGCAATTTTCTCACACTGTGTTATCTATGAGACTATCAACACCATGATGAGTTGGTT
GTCTGAATGTATAGAACTCAGAGATCAACAAAAAACTTTAAAATTATTGCTCAAAATCACCATGATCCTCATACTTGTGAACCCTA
GCAAAAGAGCACAGAAGTTCTTGCAAGGTCTGCGATACTTCATAATGGCCTTTGTTTCAGACTTCCACCATAAGCAGTTAATGGAA
AAGTTGAGGGAGGATCTCATAACAGAGCCGGAGCACCTCTTGTATAGTGTGGTGAGGAGCATTCTCAACATCATCCTGGGTGAAGG
GGTGAGCACTATGTTGACTAATAGATTTAAGTTTGTGTTAAACCTATCATACATGTGTCATTTTATAACTAAGGAAACTCCAGATA
GGTTGACAGATCAGATTAAGTGCTTTGAGAAGTATTTGGAGCCCAAGTTGGAGTTTGACAGTATTAACATCAACCCATCTGAAGAG
GGGGATGAAGATGAAAGGATGCTGCTGCTTGAATCAGCAAACAAATTTTTATCCAAAGAAACCAGTATGAGTAACAACAGAATATC
TTATAAAGTTCCTGGTGTGTCAAGAAAATTCTTCTCAATGATGACGTCTTCTTTTAACAATGGCTCTCTTTTCAAGAAAGGAGATG
ACCTAAGTGGGTTTAAAGATCCATTAGTTACTGCTGGGTGTGCAACAGCTCTTGACCTTGCAAGCAACAAAAGTGTGGTTGTGAAT
AAGTATACTGACGGAGAGAGGATACTTTATTACGATCATGATAAACTAGTGGCTGCTTCTGTTTGCCAGCTATCAGAGGTATTCCA
GAGGAAAACTAAATACCTCTTGAGTAAGGAGGATTATGATTATAAGGTGCAAAAGGCCATTAGTGACCTTGTTGTGGGGAAGAAGT
CAGGTTCCTCAAATCCCAATTCACAAGGGGCTCCTGACGAATTAGATGAGTTATTCTTGGATAGTTGTGCACTTGACTGTCTAGAG
GATGTGAAGAAATCTGTTGATGTCGTCCTTGAGAAGTATAGATATGACAGGAAGTTCCCTGTGGGAAATGGGTCAGAGGAGAAGTC
CTTGACAGACTTGAGGAAGGTTTTAGGTACTGAAGATGTGGGCTGTGTTTACTACAGACTGATCCAGGCAGAGATAGCACACCACA
TGGTGGAAGATTTTGATGAGTCACTACTACCTGGAGATGCTTATGAGATGATCTGCAAAGGCTTTTTTAAGGATTTGGAGTTAAGA
TCAAAGTATTTCTATTTGGATTCCTTGGACTCTTGCCCAATAACATGCATCACCCAAGCTGTCTCCACCAGAACATTCAATGACCA
GCAGTTTTTTCAGTGCTTCAAGTCACTACTTCTTCAGATGAATGCAGGGAAATTGGCTGGAAAATACAGCCATTACAAAAACAAAT
GCCTAAACTTCAAGATTGATAGAGAAAGGCTGATGAATGATGTTAGGATCAGTGAAAGAGAGAGCAATTCTGAGGCATTAGGTAAA
GCACTGTCATTGACAAATTGTACAACTGCAGTTCTAAAGAACCTATGTTTTTACAGTCAAGAATCCCCACAGTCATACACATCCTT
GGGTCCTGATACTGGAAGGCTCAAGTTTTCCTTATCTTACAAAGAACAAGTTGGAGGGAACAGGGAACTTTATATAGGTGACCTGA
GGACAAAAATGTTCACACGCCTAATTGAGGATTATTTTGAGGCACTAACTAAGCAATATAGAGGGAGCTGTCTTAATAATGAAAAG
GAATTCCACAATGCCATTCTAGCCATGAAATTGAATGTTTCACTAGGTCAGGTCTCTTATAGCCTCGATCACAGCAAGTGGGGGCC
TATGATGTCCCCTTTTCTTTTCCTGGTGTTTCTTCAAAATTTGCGATGGGAGACAAGAGATGATATAGAGGACATAAAAAGTAAGG
ATTACGTGTCCACTTTGCTGTCGTGGCACATTCACAAGTTAATTGAGGTACCTTTCAATGTTGTGAATGCAATGATGAGATCTTAT
CTTAAGTCTAGGTTAGGTTTGAAAAAATCACTCCACCAAACGTCAACAGAAGCTTTCTTCTTTGAATACTTTAAACAAAACAGAAT
ACCATCACATCTCAGCTCAATAATTGACATGGGGCAAGGGATCTTGCACAATGCTTCTGACTTCTACGGTCTAGTGAGTGAGAGAT
TCATAAATTATTGCATTAAGTGTCTATTTGAAGATGAAGTTGATTCATATACCTCTAGTGATGATCAAATATCACTATTTGGCAAG
GATCTTTCAGATTTACTCTCAAATGAGCCTGAGGAATTCCAAGCCATTCTAGAATTTCACTATTTCCTAAGTGATCAATTGAATAA
ATTCATCAGTCCAAAGAGTGTTATTGGTTCATTTGTTGCTGAGTTCAAATCAAGGTTTTATGTCTGGGGTGATGAAGTTCCATTGT
TAACGAAATTCGTGGCTGCCGCCCTCCACAACGTTAAGTGTAAGGAGCCACATCAATTAGCTGAAACTATTGACACTATCATTGAT
CAGTCAGTGGCCAATGGTGTGCCTGTCACACTATGTAACGCTATTCAGGAGAGAACACTGAATCTACTTAGATATGCACAATATCC
CATTGATCCTTTCTTGTTGTTTTTGGATTCTGATGTTAAAGATTGGGTTGATGGCAATAGGGGCTATAGGATTATGAGGAACATTG
AGGCAATCCTACCAGAAAGCACTCAGAAAGTTAGGAAGGTCCTAAGGACAGTTTTTAATAAGCTGAAATTAGGAGAGCTTCATGAA
GAATTCACAGCCATCTACTTGTCAGGAGACCCCGCAGATTCCTTCAAGAAACTTACCAGCCTTGTTGGTGATGACACCCTCTCAGA
AGAGGATTTATCGGTGTGTTGGCTTAATTTGACAACTCATCACCCTTTAAAGATGGTCATGAGACAGAAGGTCATTTACACAGGTG
CTGTTGAACTCGGGGAAGAAAAACTGCCTACCTTGGTGAAAACATTGCAAAGCAAGTTATCCTCTAATTTCACAAGAGGGGCACAA
AAGTTGCTCTGTGAAGCCGTCAACAAAAGTGCCTTTCAGAGTGGGATAGCATCAGGTTTCATAGGTCTTTGCAAGACACTAGGTAG
CAAATGTGTTCGATTCTCAGATAGGTCCACCGCCTATATAAAATCATTAGTTTCAAGACTGTCAGCATTGGATTCTGTTTCCAGCT
TGAAAGTTAAGGGCGTCGATCTTTGGATCTTGGGTAAGGAGCACACAAAGGCAGCTGAGGAAGCGTTAGGTTTCTTGAGACCTGTC
CTTTGGGATTACTTCTGCATAGCCTTATCTACATCACTTGAGCTGGGTTCCTGGGTGTTGGGTGAACCCAAAGTGAAGGAGAAAAC
ATCCTCAATTCCCTTCAAGCCATGTGACTATTTCCCAATGAAGCCCACTACCACAAAACTCTTGGAAGACAAGGTGGGGTTTAACC
ATATTATTCACTCATTCAGAAGACTTTACCCATCTCTATTTGAGAGACACCTCTTGCCCTTCATGAGTGACCTAGCATCAACGAAA
ATGAGGTGGACACCAAGGATTAAGTTTCTTGATCTTTGTGTGGTTCTAGATGTGAATTGTGAGGCAATGTCATTAATTTCTCATGT
TGTCAAGTGGAAGAGAGAAGAGCATTATGTGGTTCTGTCTTCAGATTTAGCAATAGCACATGAGAGGTCTCATCTCCCAATCACGG
ATGAAAGGGTGGTGACCACTTATGATGTGGTACAAAATTTCCTGAGACAAATCTACTTTGAGTCCTTCATCAGACCATTCGTTGCA
ACAAGCAGGACTTTAGGTTCTTTTACTTGGTTTCCACATAGATCTTCAATTCCTGAGTCGGAAGGGCTTGACAACCTCGGCCCCTT
TTCTTCTTTTATAGAAAAGGTTATTTATAAGGGTGTTGAAAGACCCATGTACAGGCATGATCTTTACTCAGGTTATGCTTGGCTGG
ACTTTGAATGTGCACCAGCAATTCTAAACTTAGGACAGCTCATAGCATCAGGATTAACCGAGCAGCACGTCTTTGAGTCGGTAAGT
GAGCTGCTTGAAGCTTTTGCCGACCTCAGTGTTGGGAGCGTTCAAATTTCTGTCACAGTAAATTTTCAGGTGAGAAGTCAGGGTGA
ATCATTGAAAGAGAAATTTAGTCTCCACCTCCTTTTCAAAGGGGTGGTGTTGGAAGGTGGATTATTCAAGCCTCATTCCCTTGATG
TAACTTACAGTGGTAGTGTTCAAAGATCCGCAATTAAAGATTGCTGGAGAGTTGCACAGACATCTACATGGTTTAAAAGGGAAACC
ACATCAATTTGGTTGCTGTCCACTGAAAATATTTGTGACTACTTGAGGGATAGTTCCCCCATTCCTGATGTGATACCCTTGTCCGT
CTTATTGAATGAGGAGATCCTGGACCTGGAGGAACATGATTTCACGCATATAGGGCCTGAGCATGTTGAAATCCCCTTAGTTGTTG
ACTCAGGATACCTTATTGAAGGGACCAGGAAACTCCTGCCCTTCAACCCCAACATCCATGACCAGGATCTTAATGTTTTTATTGGT
GAGCTAATGGAGGATCATTCCGAAATCTTGGAGAGATCTTTGAGCAAGATGCTGAGATCCAGAATGGACCAAGGACTACACTGGCT
ACAACTTGATATTATAGGGGTTGTGGGACGATGCATGCCTGAAGGCTACGAAAACTTCCTTACTAGAGTGTTCTCCGGAATTGACT
TCTGGGCAGATTTTAAAGGCTATAGTCTCTGCTACAGTAGATCGCAGGCTTCACTGATGATCCAGTCTTCAGAGGGGAAGTTTAGA
TTAAGAGGGAGGCTGTGCAGGCCCCTCTTTGAAGAGGTGGGGCCTCCCCTCGACATTGAGTAGGTGCTCCGAGAACAGCTCGCGCA
TCTCCCCCCGGGGGGAGCCCCGGCGGGGGGTCCCCCCGGGGGGGAGGGGAGGGGTGTTTGGGAGGGGCTTCGGGGCGGAGTTCTGC
CTCAGGGGCTGTAGGGTGGGGGATTTTGGGATGGTGGGATTTCTGGTGGTGCTGTCGGCTGGGTCTGGAGCTCTAGTCTGAAGGGT
AGTTTGTGTTTGCAGATGGGGCACCTATCTGACACTGTGTGTAAAAGTGTAAGACAGTTCAAACATAGGTAGTGATCGTTGCACCT
GACTAACCCCCTCCTTTCGAACCAGCAGCTCTTGCAGAATTCAGGGCCAGTTCCCCTGGCATCTGGTATTACTGGATGCCTTGGTT
CTGTCTCCTTGAGGAGATTTGTGCTGGGCTGTCCCTTGGACTGCGTTTTCCCCATGACTCTTGCCGGATGCGGCTTTGAGTGCAGA
ATGAAGTATCAGCAAAGATCCACAAAATTGCCTAGGATCCCCGGTGCG

### Second Polynucleotide for Guanarito Virus: MOPEVAC S GPC Guanarito (INH-95551)—SEQ ID NO: 15

5′NC region: 1-69 pb

Nucleoprotein “NP” ORF: 70-1782 pb, including possible mutated residues: D390A (1237-1239 pb), E392 (1243-1245 pb), G393 (1246-1248), H430A (1357-1359), D467A (1468-1470), H529 (1654-1656) D534 (1669-1671)

Intergenic Region (IGR): 1783-1905 pb

GPC Guanarito (INH-95551) ORF: 1906-3345 pb

3′ NC region: 3346-3398 pb

GCGCACAGTGGATCCTAGGCTAATTGATTGCGCCTTTGGAATCAGCTCTGCACCGAAAAGCCTCCAACAATGTCCAATTCAAAGGA
GGTGAAGTCCTTCTTGTGGACACAGAGCCTGAGGAGAGAACTCTCAGGGTACTGCTCCAACATAAAGATCCAAGTCATCAAGGATG
CTCAAGCACTTCTTCATGGGCTGGACTTCTCTGAAGTTGCCAATGTTCAAAGGTTGATGAGAAAGGAGAAGAGGGATGACTCTGAC
CTGAAAAGATTGAGGGACCTAAACCAGGCAGTGAACAATCTAGTTGAGTTAAAGTCAGTCCAACAGAAGAATGTTTTGAGAGTGGG
GACACTAACCTCTGATGACCTCCTCGTCCTTGCTGCCGACCTGGACAGACTCAAAGCAAAAGTCATCAGAGGTGAGAGGCCTCTTG
CTGCTGGAGTCTATATGGGCAACCTAACAGCTCAGCAGCTAGAACAGAGGAGGGTTTTGTTACAGATGGTCGGAATGGGTGGCGGG
TTCCGGGCAGGAAACACTCTCGGAGATGGCATTGTTAGAGTGTGGGATGTTCGAAACCCAGAGCTTTTAAACAATCAGTTTGGGAC
AATGCCAAGCCTGACGATTGCTTGCATGTGCAAACAAGGGCAGGCAGATCTGAATGATGTGATCCAATCGTTGTCAGACTTGGGGC
TTGTGTACACTGCAAAGTATCCAAACATGTCTGACTTAGACAAACTCTCTCAGACCCACCCAATCTTGGGGATCATTGAGCCCAAG
AAAAGTGCCATAAACATATCAGGGTACAATTTTAGCCTGTCAGCTGCGGTGAAAGCTGGTGCTTGTCTAATAGACGGCGGAAACAT
GCTGGAGACCATCAAAGTAACAAAATCCAATTTGGAAGGAATTTTGAAGGCTGCCTTGAAAGTCAAGCGTTCTTTGGGAATGTTTG
TCTCTGACACGCCAGGGGAAAGGAACCCTTATGAAAATCTCCTCTACAAACTATGTCTTTCTGGTGAGGGTTGGCCTTACATAGCA
TCAAGAACATCGATCGTCGGCAGGGCTTGGGATAACACAACTGTTGATCTGAGTGGTGATGTGCAACAGAATGCAAAGCCTGACAA
AGGTAACTCCAACAGACTCGCTCAGGCCCAAGGCATGCCTGCTGGTTTGACCTACTCTCAGACAATGGAACTCAAAGACAGCATGT
TGCAATTGGATCCAAATGCTAAGACATGGATTGCCATAGCAGCGAGACCTGAAGACCCCGTGGAGATAGCTATCTATCAACCTAAT
AATGGTCAGTATATTCATTTTTACAGGGAACCAACAGACATTAAACAATTCAAACAAGACTCCAAAGCCTCTCATGGCATTGACAT
CCAAGACCTATTCTCAGTTCAGCCGGGGTTGACAAGTGCTGTAATTGAGAGCCTGCCAAAGAACATGGTCTTGTCGTGTCAAGGTG
CTGATGCCATCAGAAAGCTTCTTGACTCCCAGAACAGGAGGGACATAAAACTGATTGATGTGTCCATGCAGAAAGACGATGCAAGA
AAATTTGAGGATAAGATCTGGGATGAATACAAACACCTTTGTAGAATGCATACGGGGATTGTAACGCAAAAGAAGAAGAGAGGTGG
CAAAGAAGAGGTGACACCACACTGTGCATTGCTGGCTTGTCTCATGTTTGAAGCAGCAGTCATAGGGAGTCCACAAATTCCAACCC
CCAGACCAGTCTTGAGTAGAGACCTGGTGTTTAGAACAGGTCCTCCCAGAGTTGTCCTGTAAGAAGGGACACCTCCACGACCCACC
GCCTCTGGCGGTGGGTCGTGGAGGCCATCAAGGAACAGTCACTCCAGGCCCCCGAGACCCACCGCCGAAGGCGGTGGGTCTCGGGG
GTGCCCCTGTTCTTCAATGCTTTCTCTTCCAGGTTACTTGTTTTCCAAGTTTTTGAAACCTGCCACACCTGCAAGCACCATTTCTA
TCGAGCCTGTGAGGCAAAGGGCACGGGTCACCCTGTATATGTCTATGTGTTGGGAACCCAACCAGGTGTAAAAAGAGACTTGTGGT
GAAAAAGATTGCACTCCAGAAACATAGATCAACCAGTGTTAGGGGAGTCTTCCCCTGCCTATCTTGGTATTCTTTGCTTAACATCT
CTGCTATTAGGTGATCACTCTCCAAGATCCACTCATTTCTAAAATCACTCTCATTCAAATAGGAACCATTACTGACCAGCCAACAC
CGAGGCAGTGAGTGCTCACCAGATTTCGTGTGGTTTATGTACCAGAATCTTGTGTAGTTGCAATATGGGACCTTCAAAATCTCCTT
CAGTTTGTTTCTCATCAATAAATTATCAGATATTAGACTATTTATTGAGTGAGTCAACATGTTGACAGCAGTTTTAGTTTGGTTGT
TCAGTTTTTCAATGGCATTCTTATTGAAATCAAACAGCCTCAACATGTCACAGAACTCAGAATCATGATTCAAATTACACTTGGCG
ACAGCTGTGTTGCCAAAGCACTTCAAATCCCCAGCAACCAACATCCACCTTTCAAGACAGTAACCACCTGGCATGTCATTACCCTT
TGGGTCAGAAAGTGACCAACTAAAAAATGCCAGTGGTTTTCTTCCAACAAGGTAACCTGCATTTTTAACAAGAAATCTCAGTGTAT
TCACATGACTATTTGAGCATTGACCTTCCCAGGTAGTGTTCCTCATAACCACAGACATTTTTGAATAGCTATCATTAGTTATGTAG
TTCCCGAATAAATGTCGGATGCCTACCTCAATCTTGCTCCTATAGTGATGGGTCTCAGGACTATCTGTTAAATTCAATTGGAATGT
AAATTCAGCATTAGTTGTCTTTTTAGGCTCACAGCAGATTCTTGGATCCCTCAAAAAGTCATGTCCCAATCCCTTAATTGTCCACC
CAAGAATCCAATCAAACTCTGGAGAGCCTTTTATACATTGAGTGTAGTTGTCGAGTATGGTTGGACTGGACGAACAGAGGACAGGG
TGCACATCCACATACTCAACTGATAATGCCTGATTACTGTTATGAGAAAGCCTAATTAGACTGTAAGAGTTGTTGACCCTACATAG
TGAAGGTAATTCATGGAAGACACCTCCTAGCTTAACTGTAAATGACTCAAAATTTGTATGATGACCAACTTTGAAAGAGCATGACC
TTCCCGCCAGAACCAAGAACACAAACAGCTGAAGAATTCCTGATTTCCAAACATTCACAATGCCCTTTATAATAGCAAGCAGTGTA
ACCACAGCCAAGGCTACATTGAGTGCTTCTTCAAAGAAGATCGGTATGTCCTGAAAGAAACTGATTAACTGTCCCATCTCCGAGAT
GCTCTAGAAATGCGCAACCAAAAAGCCTAGGATCCCCGGTGCGC

Second Polynucleotide for Carvallo virus: MOPEVAC S GPC Machupo (Carvallo)—SEQ ID NO: 16

5′NC region: 1-69 pb

Nucleoprotein “NP” ORF: 70-1782 pb, including possible mutated residues: D390A (1237-1239 pb), E392 (1243-1245 pb), G393 (1246-1248), H430A (1357-1359), D467A (1468-1470), H529 (1654-1656) D534 (1669-1671)

Intergenic Region (IGR): 1783-1905 pb

GPC ORF: 1906-3396 pb

3′ NC region: 3396-3449 pb

GCGCACAGTGGATCCTAGGCTAATTGATTGCGCCTTTGGAATCAGCTCTGCACCGAAAAGCCTCCAACAATGTCCAATTCAAAGGA
GGTGAAGTCCTTCTTGTGGACACAGAGCCTGAGGAGAGAACTCTCAGGGTACTGCTCCAACATAAAGATCCAAGTCATCAAGGATG
CTCAAGCACTTCTTCATGGGCTGGACTTCTCTGAAGTTGCCAATGTTCAAAGGTTGATGAGAAAGGAGAAGAGGGATGACTCTGAC
CTGAAAAGATTGAGGGACCTAAACCAGGCAGTGAACAATCTAGTTGAGTTAAAGTCAGTCCAACAGAAGAATGTTTTGAGAGTGGG
GACACTAACCTCTGATGACCTCCTCGTCCTTGCTGCCGACCTGGACAGACTCAAAGCAAAAGTCATCAGAGGTGAGAGGCCTCTTG
CTGCTGGAGTCTATATGGGCAACCTAACAGCTCAGCAGCTAGAACAGAGGAGGGTTTTGTTACAGATGGTCGGAATGGGTGGCGGG
TTCCGGGCAGGAAACACTCTCGGAGATGGCATTGTTAGAGTGTGGGATGTTCGAAACCCAGAGCTTTTAAACAATCAGTTTGGGAC
AATGCCAAGCCTGACGATTGCTTGCATGTGCAAACAAGGGCAGGCAGATCTGAATGATGTGATCCAATCGTTGTCAGACTTGGGGC
TTGTGTACACTGCAAAGTATCCAAACATGTCTGACTTAGACAAACTCTCTCAGACCCACCCAATCTTGGGGATCATTGAGCCCAAG
AAAAGTGCCATAAACATATCAGGGTACAATTTTAGCCTGTCAGCTGCGGTGAAAGCTGGTGCTTGTCTAATAGACGGCGGAAACAT
GCTGGAGACCATCAAAGTAACAAAATCCAATTTGGAAGGAATTTTGAAGGCTGCCTTGAAAGTCAAGCGTTCTTTGGGAATGTTTG
TCTCTGACACGCCAGGGGAAAGGAACCCTTATGAAAATCTCCTCTACAAACTATGTCTTTCTGGTGAGGGTTGGCCTTACATAGCA
TCAAGAACATCGATCGTCGGCAGGGCTTGGGATAACACAACTGTTGATCTGAGTGGTGATGTGCAACAGAATGCAAAGCCTGACAA
AGGTAACTCCAACAGACTCGCTCAGGCCCAAGGCATGCCTGCTGGTTTGACCTACTCTCAGACAATGGAACTCAAAGACAGCATGT
TGCAATTGGATCCAAATGCTAAGACATGGATTGCCATAGCAGCGAGACCTGAAGACCCCGTGGAGATAGCTATCTATCAACCTAAT
AATGGTCAGTATATTCATTTTTACAGGGAACCAACAGACATTAAACAATTCAAACAAGACTCCAAAGCCTCTCATGGCATTGACAT
CCAAGACCTATTCTCAGTTCAGCCGGGGTTGACAAGTGCTGTAATTGAGAGCCTGCCAAAGAACATGGTCTTGTCGTGTCAAGGTG
CTGATGCCATCAGAAAGCTTCTTGACTCCCAGAACAGGAGGGACATAAAACTGATTGATGTGTCCATGCAGAAAGACGATGCAAGA
AAATTTGAGGATAAGATCTGGGATGAATACAAACACCTTTGTAGAATGCATACGGGGATTGTAACGCAAAAGAAGAAGAGAGGTGG
CAAAGAAGAGGTGACACCACACTGTGCATTGCTGGCTTGTCTCATGTTTGAAGCAGCAGTCATAGGGAGTCCACAAATTCCAACCC
CCAGACCAGTCTTGAGTAGAGACCTGGTGTTTAGAACAGGTCCTCCCAGAGTTGTCCTGTAAGAAGGGACACCTCCACGACCCACC
GCCTCTGGCGGTGGGTCGTGGAGGCCATCAAGGAACAGTCACTCCAGGCCCCCGAGACCCACCGCCGAAGGCGGTGGGTCTCGGGG
GTGCCCCTGTTCTTCAATGTCTTTTGTGCCAGATGGTGGGTTTCTTCAATCTGGGATATTTGCCACATCTACAACCTCCGAAGCTG
TCCAGCTTATGAGGCAAAGGACAGGCTTCGCCTTTGAGGTGTCGATGGGTGGGTATTCCGACTAGATGAAGGAACAATGATGCTGT
GAAGAAAATTGTGCTCCAGAAACAAATATCAACTAATGTGATTGGGGTTTTGCCTTGCCTTTCAGCATATTCCTTACTTAACATCT
CTGAGATGAGGTGATCACTCTCTAAAATCCAGTCATTCCTGAATTCAGAAGTGTTAAGATAACTTCCATTCCTTATCAACCAACAT
CTTGGAAGAGTGTGCTGCCCTGTCAGGGTATGATTGACATACCAAAACTTTGTGTAATTACAATAAGGGATGCTCATTAGCTCTTT
AATTTTATTCTTCATTAACAAATTATCTGAGATTAAGGCATTCACTGTCTGGCTTAGAAGATTGATTTCTTTCTTTGATTCATCAT
TGAGGGTCTTTATTGCATTCTTGTTATAGTCGAATAGCCTCAGCATATCACAGAACTCTGAGTCATGATTTTGATTACATTTAGCA
ACAGCAGTGTTTCCGAAACATTTCATTTTGGCTGCTATCAACATCCATTCCTCTAGACAATAACCTCCTGGCATGTCCTTTCCTGA
GGAGTCTGTCAGAGACCATGAGAAAAATGCTTTCAAAGACCTCTCAAAGTTGAGATGTGTCTTACTTCTCACAAGGAAATGAAGGG
TGTTCACATGGTCAAACTGACATTTGGAAAGATGATTCACGCCACAGTAGTCAAAGGAACTGGGGTCACCACACTGATTGATGGTC
AGGTAGCACACTTTCCCTTCCTCACACGGGTCATGGAAGCCCCTGAAGAGATGCCTCATACCATTTCTTATCTTCTTTCCATACAC
TCTGGCATCATCAGCTTTGCTGATGTTGAATTGAATGTTAGATCCCTCCTTCTTTGTCTTGTTTCTACATAGCATTGGAGGATCCA
TAAGCCAGTCATGTCCCAAAGCATTTGAGAACCAATGAATAGCCCAGCTTGAGTCAGACTTGTTAAGACAATTTCCAAGGTCTTCT
GGTTCATAGATTGATACATCATACTCCTTCATGAGGACTGAAATATCAGAAACACGAATCAGGAAGGTGTTCACACCTCCCCTCAT
ATAATAAAAACTATTGTTAAGCATGCAGAGAGATGGGAGCTCATTTGAATGGTTAGCTAAAAGTCTCTGCATGGTAAGGGTGACTG
ACTGGAACTCAGTGTGTAGGCCTATTTTGAATGTGCCATCCGAGCAGGACCTCCCTGCTAGGAGGAGAAAGAAGATGAACTGGAAG
AGACCACTTTTGTAAAGGTTAATGATGCCTTTGATGACAGCTATGAGACTAACAGCCACTAAAGCGATGTTCAGAGCTTCCTGTAG
AAAAACAGGAATCTCCTGAAAGAAGCTGATAAGCTGCCCCATCTCCGAGATGCTCTAGAAATGCGCAACCAAAAAGCCTAGGATCC
CCGGTGCGC

### Second Polynucleotide for Chapare Virus: MOPEVAC S GPC Chapare—SEQ ID NO: 17

5′NC region: 1-69 pb

Nucleoprotein “NP” ORF: 70-1782 pb, including possible mutated residues: D390A (1237-1239 pb), E392 (1243-1245 pb), G393 (1246-1248), H430A (1357-1359), D467A (1468-1470), H529 (1654-1656) D534 (1669-1671)

Intergenic Region (IGR): 1783-1908 pb

GPC ORF: 1909-3363 pb

3′ NC region: 3364-3416 pb

GCGCACAGTGGATCCTAGGCTAATTGATTGCGCCTTTGGAATCAGCTCTGCACCGAAAAGCCTCCAACAATGTCCAATTCAAAGGA
GGTGAAGTCCTTCTTGTGGACACAGAGCCTGAGGAGAGAACTCTCAGGGTACTGCTCCAACATAAAGATCCAAGTCATCAAGGATG
CTCAAGCACTTCTTCATGGGCTGGACTTCTCTGAAGTTGCCAATGTTCAAAGGTTGATGAGAAAGGAGAAGAGGGATGACTCTGAC
CTGAAAAGATTGAGGGACCTAAACCAGGCAGTGAACAATCTAGTTGAGTTAAAGTCAGTCCAACAGAAGAATGTTTTGAGAGTGGG
GACACTAACCTCTGATGACCTCCTCGTCCTTGCTGCCGACCTGGACAGACTCAAAGCAAAAGTCATCAGAGGTGAGAGGCCTCTTG
CTGCTGGAGTCTATATGGGCAACCTAACAGCTCAGCAGCTAGAACAGAGGAGGGTTTTGTTACAGATGGTCGGAATGGGTGGCGGG
TTCCGGGCAGGAAACACTCTCGGAGATGGCATTGTTAGAGTGTGGGATGTTCGAAACCCAGAGCTTTTAAACAATCAGTTTGGGAC
AATGCCAAGCCTGACGATTGCTTGCATGTGCAAACAAGGGCAGGCAGATCTGAATGATGTGATCCAATCGTTGTCAGACTTGGGGC
TTGTGTACACTGCAAAGTATCCAAACATGTCTGACTTAGACAAACTCTCTCAGACCCACCCAATCTTGGGGATCATTGAGCCCAAG
AAAAGTGCCATAAACATATCAGGGTACAATTTTAGCCTGTCAGCTGCGGTGAAAGCTGGTGCTTGTCTAATAGACGGCGGAAACAT
GCTGGAGACCATCAAAGTAACAAAATCCAATTTGGAAGGAATTTTGAAGGCTGCCTTGAAAGTCAAGCGTTCTTTGGGAATGTTTG
TCTCTGACACGCCAGGGGAAAGGAACCCTTATGAAAATCTCCTCTACAAACTATGTCTTTCTGGTGAGGGTTGGCCTTACATAGCA
TCAAGAACATCGATCGTCGGCAGGGCTTGGGATAACACAACTGTTGATCTGAGTGGTGATGTGCAACAGAATGCAAAGCCTGACAA
AGGTAACTCCAACAGACTCGCTCAGGCCCAAGGCATGCCTGCTGGTTTGACCTACTCTCAGACAATGGAACTCAAAGACAGCATGT
TGCAATTGGATCCAAATGCTAAGACATGGATTGCCATAGCAGCGAGACCTGAAGACCCCGTGGAGATAGCTATCTATCAACCTAAT
AATGGTCAGTATATTCATTTTTACAGGGAACCAACAGACATTAAACAATTCAAACAAGACTCCAAAGCCTCTCATGGCATTGACAT
CCAAGACCTATTCTCAGTTCAGCCGGGGTTGACAAGTGCTGTAATTGAGAGCCTGCCAAAGAACATGGTCTTGTCGTGTCAAGGTG
CTGATGCCATCAGAAAGCTTCTTGACTCCCAGAACAGGAGGGACATAAAACTGATTGATGTGTCCATGCAGAAAGACGATGCAAGA
AAATTTGAGGATAAGATCTGGGATGAATACAAACACCTTTGTAGAATGCATACGGGGATTGTAACGCAAAAGAAGAAGAGAGGTGG
CAAAGAAGAGGTGACACCACACTGTGCATTGCTGGCTTGTCTCATGTTTGAAGCAGCAGTCATAGGGAGTCCACAAATTCCAACCC
CCAGACCAGTCTTGAGTAGAGACCTGGTGTTTAGAACAGGTCCTCCCAGAGTTGTCCTGTAAGAAGGGACACCTCCACGACCCACC
GCCTCTGGCGGTGGGTCGTGGAGGCCATCAAGGAACAGTCACTCCAGGCCCCCGAGACCCACCGCCGAAGGCGGTGGGTCTCGGGG
GTGCCCCTGTTCTTCATCAGTGTTTCCTGTGCCAGGTTGTTGGTTTCTTGAGTTCTGGATATCTGCCACACCTGCATCCACCATTG
CTGTTGAGCTTGTGAGGCAGTGGGCAAGGTTCTCCCTGAATGTGTCTGTGAGTTGGGAAGCCTACTAGATGGAGAAACAATGTTGT
TGTGAAGAACAGTGTGCTCCAAAAGCAGATGTCAACAAGTGTTATTGGGGTTTTCCCTTGTCTGTCAAAATACTCTTTGTTCAACA
TTTCGGACAACAAGTGGTCACTCTCAAGAATCCAATCATTTCTAAATTCACTTTCATTGAGGTAGCTGTTGTTTTTAACCATCCAA
CAGCGTGGAAGTGAATGCTCTCCTGTGATGGTGTGGTTGACATACCAGAATTTTGTGTAGTTGCAGTAGGGAGTGTCCAATAACTC
TTTTAATCTATTCTTCATCAAAAGATTGTCAGAAATTAAAGCATTAATGGAATGTGTTAATAGATTGACTTTGTTCTTTGTGTTGT
CATTCAATGACTCAATTGCTTTTTTGTTAAATTCAAACAGTTTCAACATGTCACAGAACTCAGAGTCATGATTTAAGTTACACTTT
GCAAGAGCAGTGTTGCCAAAACATTTCAGGTCAGATGTGACTAACATCCACCTTTCCAAACAATAACCTCCAGGCATGTCGTTGCC
AGCAGCATCTGTGATCGTCCAAGTGAACACCCCTTGCAATCTTCTTAATTTAACAGCATGCCCAGCATTAGCCATCATCAAATGCA
ACGTGTTGACATGATTGGACTTGCACTGATTCACCCAAGTCTGATTTCTGATAACTATGAACTGCTTAGGTGTTTTATCAAACATC
AATTTTGTGCCAAATAGTATGGCCAGCCTTTGGAGCATGGTTTCTTTGAAATCATGAGAACCTGCACTATCGCTCACATTTATCTG
AATGCGTGTTTCATTGACAGATGATGTCACTTTTTCACACAAGAGGGGTGGATAATTTAGAAAATCATATTTCAGAGCTCTCAAAA
TCCACTCCAATAGACCTAGAAGTTCTGATCTATGTTTAATGTCTGGGTGAACACAGTTTGATAGGTTGCCCATTACACGTGGATCA
TGTGGCGAGAGCAAGACAGACATGTCCAGTGCAGCAATCTCTACACACCATGTTGCATTCTCACTTTCACGGATGTAATAAAGAGT
TTTGTTCACTGTGCAAGAAATAGGATGGTCCTCAAAGACCTTTAACATGTTTATTGTGATGTTTTGGAGTTCTGTGGATCTTCCAA
TTTTGAATGAGCAGGACCTTCCAGCTAGAATAAGGAAGACCAGGAGTTGAAACAAACCACTTTTCCACAGATTGACCAACCCTTTT
AGGATAGCAATGAGGCTGACAGCTATTAGAGCAATGTTGATTGCTTCCTGTATGATGTTAGGAATTTCCTGAAAGAAACTCACAAG
TTGACCCATCTCCGAGATGCTCTAGAAATGCGCAACCAAAAAGCCTAGGATCCCCGGTGCGC

### Second Polynucleotide for Sabia Virus: MOPEVAC S GPC Sabia—SEQ ID NO: 18

5′NC region: 1-69 pb

Nucleoprotein “NP” ORF: 70-1782 pb, including possible mutated residues: D390A (1237-1239 pb), E392 (1243-1245 pb), G393 (1246-1248), H430A (1357-1359), D467A (1468-1470), H529 (1654-1656) D534 (1669-1671)

Intergenic Region (IGR): 1783-1905 pb

GPC ORF: 1906-3372 pb

3′ NC region: 3373-3425 pb

GCGCACAGTGGATCCTAGGCTAATTGATTGCGCCTTTGGAATCAGCTCTGCACCGAAAAGCCTCCAACAATGTCCAATTCAAAGGA
GGTGAAGTCCTTCTTGTGGACACAGAGCCTGAGGAGAGAACTCTCAGGGTACTGCTCCAACATAAAGATCCAAGTCATCAAGGATG
CTCAAGCACTTCTTCATGGGCTGGACTTCTCTGAAGTTGCCAATGTTCAAAGGTTGATGAGAAAGGAGAAGAGGGATGACTCTGAC
CTGAAAAGATTGAGGGACCTAAACCAGGCAGTGAACAATCTAGTTGAGTTAAAGTCAGTCCAACAGAAGAATGTTTTGAGAGTGGG
GACACTAACCTCTGATGACCTCCTCGTCCTTGCTGCCGACCTGGACAGACTCAAAGCAAAAGTCATCAGAGGTGAGAGGCCTCTTG
CTGCTGGAGTCTATATGGGCAACCTAACAGCTCAGCAGCTAGAACAGAGGAGGGTTTTGTTACAGATGGTCGGAATGGGTGGCGGG
TTCCGGGCAGGAAACACTCTCGGAGATGGCATTGTTAGAGTGTGGGATGTTCGAAACCCAGAGCTTTTAAACAATCAGTTTGGGAC
AATGCCAAGCCTGACGATTGCTTGCATGTGCAAACAAGGGCAGGCAGATCTGAATGATGTGATCCAATCGTTGTCAGACTTGGGGC
TTGTGTACACTGCAAAGTATCCAAACATGTCTGACTTAGACAAACTCTCTCAGACCCACCCAATCTTGGGGATCATTGAGCCCAAG
AAAAGTGCCATAAACATATCAGGGTACAATTTTAGCCTGTCAGCTGCGGTGAAAGCTGGTGCTTGTCTAATAGACGGCGGAAACAT
GCTGGAGACCATCAAAGTAACAAAATCCAATTTGGAAGGAATTTTGAAGGCTGCCTTGAAAGTCAAGCGTTCTTTGGGAATGTTTG
TCTCTGACACGCCAGGGGAAAGGAACCCTTATGAAAATCTCCTCTACAAACTATGTCTTTCTGGTGAGGGTTGGCCTTACATAGCA
TCAAGAACATCGATCGTCGGCAGGGCTTGGGATAACACAACTGTTGATCTGAGTGGTGATGTGCAACAGAATGCAAAGCCTGACAA
AGGTAACTCCAACAGACTCGCTCAGGCCCAAGGCATGCCTGCTGGTTTGACCTACTCTCAGACAATGGAACTCAAAGACAGCATGT
TGCAATTGGATCCAAATGCTAAGACATGGATTGCCATAGCAGCGAGACCTGAAGACCCCGTGGAGATAGCTATCTATCAACCTAAT
AATGGTCAGTATATTCATTTTTACAGGGAACCAACAGACATTAAACAATTCAAACAAGACTCCAAAGCCTCTCATGGCATTGACAT
CCAAGACCTATTCTCAGTTCAGCCGGGGTTGACAAGTGCTGTAATTGAGAGCCTGCCAAAGAACATGGTCTTGTCGTGTCAAGGTG
CTGATGCCATCAGAAAGCTTCTTGACTCCCAGAACAGGAGGGACATAAAACTGATTGATGTGTCCATGCAGAAAGACGATGCAAGA
AAATTTGAGGATAAGATCTGGGATGAATACAAACACCTTTGTAGAATGCATACGGGGATTGTAACGCAAAAGAAGAAGAGAGGTGG
CAAAGAAGAGGTGACACCACACTGTGCATTGCTGGCTTGTCTCATGTTTGAAGCAGCAGTCATAGGGAGTCCACAAATTCCAACCC
CCAGACCAGTCTTGAGTAGAGACCTGGTGTTTAGAACAGGTCCTCCCAGAGTTGTCCTGTAAGAAGGGACACCTCCACGACCCACC
GCCTCTGGCGGTGGGTCGTGGAGGCCATCAAGGAACAGTCACTCCAGGCCCCCGAGACCCACCGCCGAAGGCGGTGGGTCTCGGGG
GTGCCCCTGTTCTTCAGTGGTTCTTGTGCCAGGTGATTGGCTTTTTTAGTTCAGGGTATTTCCCACATCTACATCCTCCTCTACTG
TTGAGCCTATGGGGTAGTGGGCAGGGTTCACCACGTATGTGTCTATGAGTTGGAAAGCCTACCAGGTGAAGAAACAGTGTTGTTGT
GAAAAACAAAGTGCTCCAGAAACAGATATCCACCAAAGTCAACGGTGTCTTTCCCTGTCTATCTATGTATTCTTTATTGAGCATTT
CAGACAATAAATGATCACTCTCAATAATCCAATCATTTCTAAATTCACTTTCATTCAAGTAGCTATTATTTCTAACAAGCCAGCAC
CGTGGCAATGAGTGTTCCCCTGATGCTGTGTGATTGACATACCAAAATTTGGTGTAATTACAATAAGGAGTGTTCAACAATTCTTT
AAGTCGATTCTTCATCAGTAAGTTGTCAGATATTAATGCATTAATTGAGTGGGTCAGCAAGTTTACCTTGTTTTTTGTATTGTCAT
TCAATGTCTCTATCGCTTTTTTGTTGAACTCAAACAATTTCAACATGTCACAGAATTCCGAATCGTGGTCAAGGTTACATTTTGCT
AGTGCTGTGTTTCCAAAGCACTTAAGATCTGACGTCACTAGCATCCATCTTTCAAGACAATAACCACCAGGCATGTCATTGCCCAC
TGCATCAGTTATTGTCCAGGAGAAAATGCCGAGTGGTCTTCTAGAACCAGAAGAGCGACCAGCATTCGCCAACATTAAATGCATGG
AGTTTACATGATTCATTTCGCATTGATTTTTCCAAGTTGAATTTCTAATCAATAAAAACCTTTTTTTACCTAAATCCTGGATATTT
GAAAATGCAATTCTTGAACCGAATAGAACCCCTAGTCTTTGAAGGATGGTATCTTCAAACCCGTGAGACCCAAACCCCTCAGTGAT
GTTTATCTGCACACGTGTTTCATTCACTGTTGAAGTTTGCTTTTGACACAACGGTGTTGGATCATGATTGAAGTCATACTTTAGGG
CTCTAAAAATCCACTCAAGTAAGCCAACCATCCTGCTTCTGTGCTCAACTGCAGGGTGCACACAGTTTGACAGATTGTTGAGGACT
TGACGATCATGTTCAGCCATGAGCAGGGTAACATCAGTCACAGACACCTCAAGACACCAAGTGGCATTTTTGTTTTCATGGACATA
GTAGGTGGAATGATTCACCATGCAAGATGTGGGGTGGTCCTCGAATACCTTCAACATATCAAACGTTATGTTTTGCAATTCTGTGC
TCCTTCCAATTCTAAAAGAACACGATCTTCCTGCTAGAGTCAAAAAGAATATCAATTGGAAAAGGCCACTCTTCCACAAGTTAATC
ATCCCTTTCAAGGCAGCAATTAAGCTCACTGCTATCAGAGCTATGTTGATAGCCTCATGGATGATATTCGGAACTTCTTCAAAAAA
GCTGAACAATTGACCCATCTCCGAGATGCTCTAGAAATGCGCAACCAAAAAGCCTAGGATCCCCGGTGCGC

### Second Polynucleotide for Junin Virus: MOPEVAC S GPC Junin—SEQ ID NO: 19

5′NC region: 1-69 pb

Nucleoprotein “NP” ORF: 70-1782 pb, including possible mutated residues: D390A (1237-1239 pb), E392 (1243-1245 pb), G393 (1246-1248), H430A (1357-1359), D467A (1468-1470), H529 (1654-1656) D534 (1669-1671)

Intergenic Region (IGR): 1783-1905 pb

GPC ORF: 1906-3363 pb

3′ NC region: 3364-3416 pb

GCGCACAGTGGATCCTAGGCTAATTGATTGCGCCTTTGGAATCAGCTCTGCACCGAAAAGCCTCCAACAATGTCCAATTCAAAGGA
GGTGAAGTCCTTCTTGTGGACACAGAGCCTGAGGAGAGAACTCTCAGGGTACTGCTCCAACATAAAGATCCAAGTCATCAAGGATG
CTCAAGCACTTCTTCATGGGCTGGACTTCTCTGAAGTTGCCAATGTTCAAAGGTTGATGAGAAAGGAGAAGAGGGATGACTCTGAC
CTGAAAAGATTGAGGGACCTAAACCAGGCAGTGAACAATCTAGTTGAGTTAAAGTCAGTCCAACAGAAGAATGTTTTGAGAGTGGG
GACACTAACCTCTGATGACCTCCTCGTCCTTGCTGCCGACCTGGACAGACTCAAAGCAAAAGTCATCAGAGGTGAGAGGCCTCTTG
CTGCTGGAGTCTATATGGGCAACCTAACAGCTCAGCAGCTAGAACAGAGGAGGGTTTTGTTACAGATGGTCGGAATGGGTGGCGGG
TTCCGGGCAGGAAACACTCTCGGAGATGGCATTGTTAGAGTGTGGGATGTTCGAAACCCAGAGCTTTTAAACAATCAGTTTGGGAC
AATGCCAAGCCTGACGATTGCTTGCATGTGCAAACAAGGGCAGGCAGATCTGAATGATGTGATCCAATCGTTGTCAGACTTGGGGC
TTGTGTACACTGCAAAGTATCCAAACATGTCTGACTTAGACAAACTCTCTCAGACCCACCCAATCTTGGGGATCATTGAGCCCAAG
AAAAGTGCCATAAACATATCAGGGTACAATTTTAGCCTGTCAGCTGCGGTGAAAGCTGGTGCTTGTCTAATAGACGGCGGAAACAT
GCTGGAGACCATCAAAGTAACAAAATCCAATTTGGAAGGAATTTTGAAGGCTGCCTTGAAAGTCAAGCGTTCTTTGGGAATGTTTG
TCTCTGACACGCCAGGGGAAAGGAACCCTTATGAAAATCTCCTCTACAAACTATGTCTTTCTGGTGAGGGTTGGCCTTACATAGCA
TCAAGAACATCGATCGTCGGCAGGGCTTGGGATAACACAACTGTTGATCTGAGTGGTGATGTGCAACAGAATGCAAAGCCTGACAA
AGGTAACTCCAACAGACTCGCTCAGGCCCAAGGCATGCCTGCTGGTTTGACCTACTCTCAGACAATGGAACTCAAAGACAGCATGT
TGCAATTGGATCCAAATGCTAAGACATGGATTGCCATAGCAGCGAGACCTGAAGACCCCGTGGAGATAGCTATCTATCAACCTAAT
AATGGTCAGTATATTCATTTTTACAGGGAACCAACAGACATTAAACAATTCAAACAAGACTCCAAAGCCTCTCATGGCATTGACAT
CCAAGACCTATTCTCAGTTCAGCCGGGGTTGACAAGTGCTGTAATTGAGAGCCTGCCAAAGAACATGGTCTTGTCGTGTCAAGGTG
CTGATGCCATCAGAAAGCTTCTTGACTCCCAGAACAGGAGGGACATAAAACTGATTGATGTGTCCATGCAGAAAGACGATGCAAGA
AAATTTGAGGATAAGATCTGGGATGAATACAAACACCTTTGTAGAATGCATACGGGGATTGTAACGCAAAAGAAGAAGAGAGGTGG
CAAAGAAGAGGTGACACCACACTGTGCATTGCTGGCTTGTCTCATGTTTGAAGCAGCAGTCATAGGGAGTCCACAAATTCCAACCC
CCAGACCAGTCTTGAGTAGAGACCTGGTGTTTAGAACAGGTCCTCCCAGAGTTGTCCTGTAAGAAGGGACACCTCCACGACCCACC
GCCTCTGGCGGTGGGTCGTGGAGGCCATCAAGGAACAGTCACTCCAGGCCCCCGAGACCCACCGCCGAAGGCGGTGGGTCTCGGGG
GTGCCCCTGTTCTTCAGTGTCTTCTACGCCAAACTGTTGGTTTCTTTAGATTGGGGTACTTACCACATCTGCAACCACCCAAGCTG
TTCAACCTGTGTGGCAAAGGGCATGCTTCGCCCCTGATGTGTCTGTGGGTAGGTATGCCCACCAAATGAAGGAAGAGTGACGCTGT
GAAAAATACTGTGCTCCAAAAACAGATGTCAACTAAAGTCAAAGGAGTTTTACCCTGTCTGTCCGAATACTCTTTGCTTAGCATTT
CAGAAATTAAGAAGTCACTCTCCAATATCCAGTCATTACGGAAGTCAGAGATGTTCAAATAGCTGTTGTTTTTTATTAACCAGCAC
CTTGGTAATGAGTGTTGTCCTGAAAGTGTGTGGTTGACATACCAAAATTTTGTGTAATTGCAGTAGGGGACACTCATCAGTTCCCT
AATTTTGTTTTTCATCAATAAATTGTCAGATATCAAGGCATTGATTGTCTGCCCCATCAGATTTACTTGTTTCTTTGTTTCATCAT
TTAGGGTTTTGATGGCATTTTTGTTGTAATCAAAAAGCCTCAACATGTCACAGAATTCAGAGTCATGATTCAAATTGCATTTTGCT
ACAGCAGTATTGCCAAAACACTTCATTTTGGCTGCCACGAGCATCCACTCTTCCAGACAATAGCCTCCAGGGGTATCCTTGCCGGA
TGAGTCTGTCAAAGACCAGGAGAAGAATGCTTTCAAGGACCTCCTTGGAAGTTGAATATTTTTGCCTCTTGTAAGGAAATGTAATG
TGTTAACATGGTCGAGTGGACATTGGAGAGGCCAACTGGTGGGTTGTGCCTTCATTAAACACAGTTTGCCATTCAAGCAAGAGTCA
GGATATTCTCTATATAAATGATGCATGCCAGTCTTAAACTTCTTAGCATAGTTTTCATTAACACCAGTCTTGGAGGTGTTGACTTG
AAAGATGAAGCCTTCTGTCTTTGTACGGTTCCTACACAGAAATGGTGGGTCTAGATGCCAATCATGTCCCACAGCATTCATGAACC
ATTGAGACAACCAAATTTGATCATCACTTTTAGAACACCAGCTCATATCTGCCGGATGTTGTATTATAACATCATACTGTGGCAAC
AGTACTTCAATATCATCAAAGCTGATCATAAATGAAGCATTGCCCCCCTTAATGTAAAGATGGCTCTTGTTTAAGGTACACAACAA
AGGTAGGTCATGTGGATTGTTGGAAAAGAGACCCACCATTGAGAAGGACACAGTCTGGAACTCAGTATGCAGTCCGATTTTGAAAG
CTTCTTCTGTGCAGGATCTTCCTGCAAGCGCTAGGAATACAAAGAATTGGAATAAACCACTTTTGTACAAGTTCACTATCCCCTTA
ATGATGGCAATGAGACTGACTGCTACAAGAGCAATGTTCAGAGCCTCCTGCAAAAAAGTTGGTATTTCTTGCATGAAGCTAATGAA
CTGCCCCATCTCCGAGATGCTCTAGAAATGCGCAACCAAAAAGCCTAGGATCCCCGGTGCGC

### Third Polynucleotide: MOPEIA RNA Dependant RNA Polymerase “LpoI”—Accession number AEO89358.1—SEQ ID NO: 20

ATGGAGGAGTTGCTGTCCGAGAGCAAAGATCTTGTGAGCAGGTACCTCTTAGAGGATGAGAGGCTTTCAAAACAGAAGCTAGCCTT
CCTAGTTCAGACAGAGCCGAGAATGCTATTAATTGAGGGCCTTAAGTTGTTGTCACTCTGTATTGAGATAGACAGTTGCAAAGCCA
ATGGTTGTGAGCACAACTCAGAAGACCTTTCTGTTGAAATCCTTCTGCAGAGACAAGGTGTTCTGTGCCCTGGTCTACCATTTGTG
GTGCCAGATGGATTCAAATTCAGTGGGAACACATTAATATTACTGGAATGCTTTGTCAGGACTTCACCAATAAACTTTGAGCAAAA
GTATAAAGAAGATACCATTAAGCTAGAGTCTTTAAAGCCTGACTTATCATCTGTTGACATCATTCTGTTGCCCTTGATAGACGGCA
GAACCAATTTCTACACTGATTTGTTTCCAGAATGGGCCAATGAAAGGTTTAGGCATATCCTATTTAGCTTACTTGAATTCTCGCAG
CAGTCATCAAAAATGTTTGAAGAATCTGAGTACTCAAGGCTTTGTGAATCTTTAACAAAAGCAGGAGTGAGAACATCGGGCATTGA
GAGCCTCAATGTTTTGACAGATTCTCGATCTGATCATTATGAGAGGGTGTTGGAGCTCTGCCACAGAGGCATCAATAACAAAATGT
CCATTCTTGATGTGAAAAAAGAGATTGTATCAGAATTTCATGCTTTTAGGAATAAGCTTAAAGAGGGTGAGATAGAAAGACAATTT
GTCAGGACAGATAGGCGACAGCTCTTGAGAGATTTCAACAACCTTTATATTGACAGAGAGGGGGACACACCCTCAGAGATTGATCC
TCTAAAAGAGAGGTTTGTAAAATCCTCGCCTATGGTAACAGCGCTCTATGGTGACTACGACCGTTATAGGCAAGAGGGAGTTGATC
GAGACAGCTGCTTGCAGAATCACTTCCAAAGCTCTGTGCCTGGATGGAAGTCACTGTTGAATAAAATAAAATCACTAAAGTTATTG
AACACCAGAAGAAAACTAATGCTGACTTTTGACGCAATCATCCTTTTGGCCCACTTAAAGGATCTTAAATGTCACGGCGAGCTGTT
AGGATCAGAATGGCTTGGCTCCTCATTCTTAAGTGTGAACGACAGATTGGTGTCACTACAAGAAACACAAAAGGACCTTAAAAAGT
GGATTGAAAGGAGAATGGTGAGTGCAATGAAGAAGAAGGGAGGTGTAGGAACTCTGTGTCAGAGATCTGAGCTTATATTCTTTGAC
ATCATAAACAAACTCCTCACAAAGGCCAAAGAGGCATTATCCTCTGCCAGTTTGTGCTTTAGAGATTATGTTAAAGAGGAAGATAT
ACTGGAGGAGGACAGTTACGAGAGGCTTATGTTAATGGAGAAAAGAGGGATTCAGCCAACAATGAGCTATGAGAAAGAAGAGGGGA
ATCAATTCCCCTACCCTCTTATTGAGTTGGAAGCTGATTCCATAGAAGACCTGAGAAGACTATCTAGCATCTCTTTGGCATTGGTG
AATTCAATGAAGACATCATCAGTAGCCAAAGTGAGGCAAAATGAGTATGGTGCTGCAAGGTACAAACGTGTACGTTGTAAGGAAGC
TTTTAATCAAAGCTTTATCATGGGAAGCGGGAATTTCAACTTAATTTATCAGAAAACAGGAGAGTGCTCAAAATGTTATGCCATTA
ACAATCCTGAGAAGGGGGAGATTTGTTCATTCTATGCAGATCCAAAAAGGTTTTTTCCTGCAATTTTCTCACACTGTGTTATCTAT
GAGACTATCAACACCATGATGAGTTGGTTGTCTGAATGTATAGAACTCAGAGATCAACAAAAAACTTTAAAATTATTGCTCAAAAT
CACCATGATCCTCATACTTGTGAACCCTAGCAAAAGAGCACAGAAGTTCTTGCAAGGTCTGCGATACTTCATAATGGCCTTTGTTT
CAGACTTCCACCATAAGCAGTTAATGGAAAAGTTGAGGGAGGATCTCATAACAGAGCCGGAGCACCTCTTGTATAGTGTGGTGAGG
AGCATTCTCAACATCATCCTGGGTGAAGGGGTGAGCACTATGTTGACTAATAGATTTAAGTTTGTGTTAAACCTATCATACATGTG
TCATTTTATAACTAAGGAAACTCCAGATAGGTTGACAGATCAGATTAAGTGCTTTGAGAAGTATTTGGAGCCCAAGTTGGAGTTTG
ACAGTATTAACATCAACCCATCTGAAGAGGGGGATGAAGATGAAAGGATGCTGCTGCTTGAATCAGCAAACAAATTTTTATCCAAA
GAAACCAGTATGAGTAACAACAGAATATCTTATAAAGTTCCTGGTGTGTCAAGAAAATTCTTCTCAATGATGACGTCTTCTTTTAA
CAATGGCTCTCTTTTCAAGAAAGGAGATGACCTAAGTGGGTTTAAAGATCCATTAGTTACTGCTGGGTGTGCAACAGCTCTTGACC
TTGCAAGCAACAAAAGTGTGGTTGTGAATAAGTATACTGACGGAGAGAGGATACTTAATTACGATCATGATAAACTAGTGGCTGCT
TCTGTTTGCCAGCTATCAGAGGTATTCCAGAGGAAAACTAAATACCTCTTGAGTAAGGAGGATTATGATTATAAGGTGCAAAAGGC
CATTAGTGACCTTGTTGTGGGGAAGAAGTCAGGTTCCTCAAATCCCAATTCACAAGGGGCTCCTGACGAATTAGATGAGTTATTCT
TGGATAGTTGTGCACTTGACTGTCTAGAGGATGTGAAGAAATCTGTTGATGTCGTCCTTGAGAAGTATAGATATGACAGGAAGTTC
CCTGTGGGAAATGGGTCAGAGGAGAAGTCCTTGACAGACTTGAGGAAGGTTTTAGGTACTGAAGATGTGGGCTGTGTTTACTACAG
ACTGATCCAGGCAGAGATAGCACACCACATGGTGGAAGATTTTGATGAGTCACTACTACCTGGAGATGCTTATGAGATGATCTGCA
AAGGCTTTTTTAAGGATTTGGAGTTAAGGTCAAAGTATTTCTATTTGGATTCCTTGGACTCTTGCCCAATAACATGCATCACCCAA
GCTGTCTCCACCAGAACATTCAATGACCAGCAGTTTTTTCAGTGCTTCAAGTCACTACTTCTTCAGATGAATGCAGGGAAATTGGC
TGGAAAATACAGCCATTACAAAAACAAATGCTTAAACTTCAAGATTGATAGAGAAAGGCTGATGAATGATGTTAGGATCAGTGAAA
GAGAGAGCAATTCTGAGGCATTAGGTAAAGCACTGTCATTGACAAATTGTACAACTGCAGTTCTAAAGAACCTATGTTTTTACAGT
CAAGAATCCCCACAGTCATACACATCCTTGGGTCCTGATACTGGAAGGCTCAAGTTTTCCTTATCTTACAAAGAACAAGTTGGAGG
GAACAGGGAACTTTATATAGGTGACCTGAGGACAAAAATGTTCACACGCCTAATTGAGGATTATTTTGAGGCACTAACTAAGCAAT
ATAGAGGGAGCTGTCTTAATAATGAAAAGGAATTCCACAATGCCATTCTAGCCATGAAATTGAATGTTTCACTAGGTCAGGTCTCT
TATAGCCTCGATCACAGCAAGTGGGGGCCTATGATGTCCCCTTTTCTTTTCCTGGTGTTTCTTCAAAATTTGCGATGGGAGACAGG
AGATGATATAGAGGACATAAAAAGTAAGGATTACGTGTCCACTTTGCTGTCGTGGCACATTCACAAGTTAATTGAGGTACCTTTCA
ATGTTGTGAATGCAATGATGAGATCTTATCTTAAGTCTAGGTTAGGTTTGAAAAAATCACTCCACCAAACGTCAACAGAAGCTTTC
TTCTTTGAATACTTTAAACAAAACAGGATACCATCACATCTCAGCTCAATAATTGACATGGGGCAAGGGATCTTGCACAATGCTTC
TGACTTCTACGGTCTAGTGAGTGAGAGATTCATAAATTATTGCATTAAGTGTCTATTTGAAGATGAAGTTGATTCATATACCTCTA
GTGATGATCAAATATCACTATTTGGCAAGGATCTTTCAGATTTACTCTCAAATGAGCCTGAGGAATTCCAAGCCATTCTAGAATTT
CACTATTTCCTAAGTGATCAATTGAATAAATTCATCAGTCCAAAGAGTGTTATTGGTTCATTTGTTGCTGAGTTCAAATCAAGGTT
TTATGTCTGGGGTGATGAAGTTCCATTGTTAACGAAATTCGTGGCTGCCGCCCTCCACAACGTTAAGTGTAAGGAGCCACATCAAT
TAGCTGAAACTATTGACACTATCATTGATCAGTCAGTGGCCAATGGTGTGCCTGTCACACTATGTAACGCTATTCAGGAGAGAACA
CTGAATCTACTTAGATATGCACAATATCCCATTGATCCTTTCTTGTTGTTTTTGGATTCTGATGTTAAAGATTGGGTTGATGGCAA
TAGGGGCTATAGGATTATGAGGAACATTGAGGCAATCCTACCAGAAAGCACTCAGAAAGTTAGGAAGGTCCTAAGGACAGTTTTTA
ATAAGCTGAAATTAGGAGAGCTTCATGAAGAATTCACAGCCATCTACTTGTCAGGAGACCCCGCAGATTCCTTCAAGAAACTTACC
AGCCTTGTTGGTGATGACACCCTCTCAGAAGAGGATTTATCGGTGTGTTGGCTTAATTTGACAACTCATCACCCTTTAAAGATGGT
CATGAGACAGAAGGTCATTTACACAGGTGCTGTTGAACTCGGGGAAGAAAAACTGCCTACCTTGGTGAAAACATTGCAAAGCAAGT
TATCCTCTAATTTCACAAGAGGGGCACAAAAGTTGCTCTGTGAAGCCGTCAACAAAAGTGCCTTTCAGAGTGGGATAGCATCAGGT
TTCATAGGTCTTTGCAAGACACTAGGTAGCAAATGTGTTCGATTCTCAGATAGGTCCACCGCCTATATAAAATCATTAGTTTCAAG
ACTGTCAGCATTGGATTCTGTTTCCAGCTTGAAAGTTAAGGGCGTCGATCTTTGGATCTTGGGTAAGGAGCACACAAAGGCAGCTG
AGGAAGCGTTAGGTTTCTTGAGACCTGTCCTTTGGGATTACTTCTGCATAGCCTTATCTACATCACTTGAGCTGGGTTCCTGGGTG
TTGGGTGAACCCAAAGTGAAGGAGAAAACATCCTCAATTCCCTTCAAGCCATGTGACTATTTCCCAATGAAGCCCACTACCACAAA
ACTCTTGGAAGACAAGGTGGGGTTTAACCATATTATTCACTCATTCAGAAGACTTTACCCATCTCTATTTGAGAGACACCTCTTGC
CCTTCATGAGTGACCTAGCATCAACGAAAATGAGGTGGACACCAAGGATTAAGTTTCTTGATCTTTGTGTGGTTCTAGATGTGAAT
TGTGAGGCAATGTCATTAATTTCTCATGTTGTCAAGTGGAAGAGAGAAGAGCATTATGTGGTTCTGTCTTCAGATTTAGCAATAGC
ACATGAGAGGTCTCATCTCCCAATCACGGATGAAAGGGTGGTGACCACTTATGATGTGGTACAAAATTTCCTGAGACAAATCTACT
TTGAGTCCTTCATCAGACCATTCGTTGCAACAAGCAGGACTTTAGGTTCTTTTACTTGGTTTCCACATAGATCTTCAATTCCTGAG
TCGGAAGGGCTTGACAACCTCGGCCCCTTTTCTTCTTTTATAGAAAAGGTTATTTATAAGGGTGTTGAAAGACCCATGTACAGGCA
TGATCTTTACTCAGGTTATGCTTGGCTGGATTTTGAATGTGCACCAGCAATTCTAAACTTAGGACAGCTCATAGCATCAGGATTAA
CCGAGCAGCACGTCTTTGAGTCGGTAAGTGAGCTGCTTGAAGCTTTTGCCGACCTCAGTGTTGGGAGCGTTCAAATTTCTGTCACA
GTAAATTTTCAGGTGAGAAGTCAGGGTGAATCATTGAAAGAGAAATTTAGTCTCCACCTCCTTTTCAAAGGGGTGGTGTTGGAAGG
TGGATTATTCAAGCCTCATTCCCTTGATGTAACTTACAGTGGTAGTGTTCAAAGATCCGCAATTAAAGATTGCTGGAGAGTTGCAC
AGACATCTACATGGTTTAAAAGGGAAACCACATCAATTTGGTTGCTGTCCACTGAAAATATTTGTGACTACTTGAGGGATAGTTCC
CCCATTCCTGATGTGATACCCTTGTCCGTCTTATTGAATGAGGAGATCCTGGACCTGGAGGAACATGATTTCACGCATATAGGGCC
TGAGCATGTTGAAATCCCCTTAGTTGTTGACTCAGGATACCTTATTGAAGGGACCAGGAAACTCCTGCCCTTCAACCCCAACATCC
ATGACCAGGATCTTAATGTTTTTATTGGTGAGCTAATGGAGGATCATTCCGAAATCTTGGAGAGATCTTTGAGCAAGATGCTGAGA
TCCAGAATGGACCAAGGACTACACTGGCTACAACTTGATATTATAGGGGTTGTGGGACGATGCATGCCTGAAGGCTACGAAAACTT
CCTTACTAGAGTGTTCTCCGGAATTGACTTCTGGGCAGATTTTAAAGGCTATAGTCTCTGCTACAGTAGATCGCAGGCTTCACTGA
TGATCCAGTCTTCAGAGGGGAAGTTTAGATTAAGAGGGAGGCTGTGCAGGCCCCTCTTTGAAGAGGTGGGGCCTCCCCTCGACATT
GAGTAG

### Fourth Polynucleotide: MOPEIA Nucleoprotein “NP” WT—Accession number AEO89356.1—SEQ ID NO: 21

ATGTCCAATTCAAAGGAGGTGAAGTCCTTCTTGTGGACACAGAGCCTGAGGAGAGAACTCTCAGGGTACTGCTCCAACATAAAGAT
CCAAGTCATCAAGGATGCTCAAGCACTTCTTCATGGGCTGGACTTCTCTGAAGTTGCCAATGTTCAAAGGTTGATGAGAAAGGAGA
AGAGGGATGACTCTGACCTGAAAAGATTGAGGGACCTAAACCAGGCAGTGAACAATCTAGTTGAGTTAAAGTCAGTCCAACAGAAG
AATGTTTTGAGAGTGGGGACACTAACCTCTGATGACCTCCTCGTCCTTGCTGCCGACCTGGACAGACTCAAAGCAAAAGTCATCAG
AGGTGAGAGGCCTCTTGCTGCTGGAGTCTATATGGGCAACCTAACAGCTCAGCAGCTAGAACAGAGGAGGGTTTTGTTACAGATGG
TCGGAATGGGTGGCGGGTTCCGGGCAGGAAACACTCTCGGAGATGGCATTGTTAGAGTGTGGGATGTTCGAAACCCAGAGCTTTTA
AACAATCAGTTTGGGACAATGCCAAGCCTGACGATTGCTTGCATGTGCAAACAAGGGCAGGCAGATCTGAATGATGTGATCCAATC
GTTGTCAGACTTGGGGCTTGTGTACACTGCAAAGTATCCAAACATGTCTGACTTAGACAAACTCTCTCAGACCCACCCAATCTTGG
GGATCATTGAGCCCAAGAAAAGTGCCATAAACATATCAGGGTACAATTTTAGCCTGTCAGCTGCGGTGAAAGCTGGTGCTTGTCTA
ATAGACGGCGGAAACATGCTGGAGACCATCAAAGTAACAAAATCCAATTTGGAAGGAATTTTGAAGGCTGCCTTGAAAGTCAAGCG
TTCTTTGGGAATGTTTGTCTCTGACACGCCAGGGGAAAGGAACCCTTATGAAAATCTCCTCTACAAACTATGTCTTTCTGGAGAGG
GTTGGCCTTACATAGCATCAAGAACATCGATCGTCGGCAGGGCTTGGGATAACACAACTGTTGATCTGAGTGGTGATGTGCAACAG
AATGCAAAGCCTGACAAAGGTAACTCCAACAGACTCGCTCAGGCCCAAGGCATGCCTGCTGGTTTGACCTACTCTCAGACAATGGA
ACTCAAAGACAGCATGTTGCAATTGGATCCAAATGCTAAGACATGGATTGACATAGAAGGGAGACCTGAAGACCCCGTGGAGATAG
CTATCTATCAACCTAATAATGGTCAGTATATTCATTTTTACAGGGAACCAACAGACATTAAACAATTCAAACAAGACTCCAAACAC
TCTCATGGCATTGACATCCAAGACCTATTCTCAGTTCAGCCGGGGTTGACAAGTGCTGTAATTGAGAGCCTGCCAAAGAACATGGT
CTTGTCGTGTCAAGGTGCTGATGACATCAGAAAGCTTCTTGACTCCCAGAACAGGAGGGACATAAAACTGATTGATGTGTCCATGC
AGAAAGACGATGCAAGAAAATTTGAGGATAAGATCTGGGATGAATACAAACACCTTTGTAGAATGCATACGGGGATTGTAACGCAA
AAGAAGAAGAGAGGTGGCAAAGAAGAAGTGACACCACACTGTGCATTGCTGGATTGTCTCATGTTTGAAGCAGCAGTCATAGGGAG
TCCACAAATTCCAACCCCCAGACCAGTCTTGAGTAGAGACCTGGTGTTTAGAACAGGTCCTCCCAGAGTTGTCCTGTAA

### Kinetic of Replication

Supernatants from VeroE6 cells, infected at an moi of 0.0001 with the different MOPEVAC viruses, were collected every day until day 7. Infectious particles were then quantified by virus titration. Samples were serially diluted in DMEM, 2% FCS, added to VeroE6 cells, and the plates incubated for 1 h at 37° C. Medium supplemented with carboxymethylcellulose was added and the plates incubated for one week. Cells were then fixed for 20 min with 4% formaldehyde. To determine the number of focus-forming units, plates were permeabilized for 5 min with 0.5% triton, stained for 1 h with anti-virus antibody, and for an addition hour with HRP-conjugated secondary antibody. The reaction was finally revealed using NBT/BCIP (Thermo Scientific). Focus-forming units were counted.

### Stability Experiments

For stability experiments, Vero E6 cells were used as described above. The stability was tested until passage 10, starting from passage 2. After each passage, viral RNAs harvested in supernatants at day 4 were quantified by RT-qPCR. VeroE6 cells were then infected with the supernatant using ten copies of genome per cell. The infectious titers in supernatants were quantified as described above. The viruses harvested in supernatants at passages 2, 5, and 10 were sequenced on MiniSeq (Illumina) and analyzed using the public platform Galaxy48. Briefly, RNA was extracted from 1 ml of supernatant with the QIAamp Viral RNA Mini Kit (Qiagen) according to manufacturer's instructions. The RNAs were rigorously treated with Turbo DNase (Ambion, Thermofisher) and concentrated by ethanol precipitation. Then, cytoplasmic and mitochondrial ribosomal RNAs were removed using the NEBNext® rRNA depletion kit v2 (human/mouse/rat). The libraries were prepared using the NEBNext® ultra II RNA library prep for illumina® with 6 minutes of RNA fragmentation and 16 cycles of amplification. Finally, quality and the concentration of libraries were determined by using the High Sensitivity D5000 Screentape assay on a TapeStation (Agilent). Sequencing was performed using an illumina Miniseq platform with 150-base paired ends and single indexing for each library. The loading concentration on the flow cell for the sequencing was 1.45 pM from a pool of normalized concentration of 18 libraries. For data analysis, reads were trimmed according to the quality score (99%) and length (reads below 80 bp were removed) and illumina adapter were deleted using trimmomatic V0.38. Trimmed fastq files were then mapped onto the genome of rescued viruses using bowtie2 V2.4.5 and PCR duplicates were removed using MarkDuplicates. Finally, consensus sequences were called by using ivar consensus and variants were checked on Integrative Genomics Viewer.

### IgG Detection on 293T Cells

293T cells were transfected in 12-well plates with phCMV plasmids coding for GPC gene or the empty vector using Lipofectamine 2000 (Invitrogen). After 2 days of incubation, transfected cells were harvested and divided into 96-well plates. Cells were incubated with Live Dead fixable viability dye (Life Technologies) and plasma samples diluted 1/20 in PBS, 2.5% FCS and 2 mM EDTA for 30 min in ice. After two washes in the same buffer, secondary antibody anti-monkey IgG FITC (Southern Biotech) was added to the cells for 30 min at +4° C. Two final washes were performed before fixation with paraformaldehyde 2% and analysis by flow cytometry (Fortessa 4 L, BD). The percentage of cells that have bound anti-GPC antibodies was determined on live cells (Kaluza software for flow cytometry analysis).

### Results

### MOPEVAC_{MACV} Vaccination

Previous experiment demonstrated the ability of a single dose of MOPEVAC_{LASV} to protect cynomolgus monkeys against a lethal challenge with LASV (1). At the end of the immunization period, the antibody response was more sustained than the one obtained with a measles/LASV vaccine. IgG specific of the virus and neutralizing antibodies (Nabs) were more expressed. Humoral response seems to be important in the protection against NW arenaviruses and JUNV Nabs titers seem to be critical in the control of infection in Argentine hemorrhagic fever patients²⁻⁴. We thus hypothesized that MOPEVAC would be an efficient vaccine platform to protect against NW arenaviruses and constructed the MOPEVAC_{MACV}. Eleven cynomolgus monkeys were vaccinated (n=8) or not (n=3) with this virus. Unvaccinated controls received two injections of the vehicle, four animals were vaccinated with a single dose of the vaccine at day 30 and a prime/boost strategy was used in four animals with injections at days 0 and 30. All were challenged with MACV at day 67. Animals were monitored each day and sampled periodically (FIG. 1a) to evaluate their clinical state and to obtain samples to assess hematological, biological, virological and immunological parameters. The MOPEVACMACY consists in a hyper attenuated live Mopeia virus: the exonucleasic function is abolished and it carries the GPC gene of MACV in place of its own GPC (5) (FIG. 1b). The antibody production in response to the vaccine was evaluated by ELISA and seroneutralization (FIG. 1c). We could detect IgG specific of MACV from day 9 post immunization and the antibody titer raised up to 1,000 during the first month. The animals that received a second vaccine injection experienced a rapid and intense reactivation of the antibody production from day 2 after the boost. The IgG titer reached 16,000, the limit in our test. The IgG response was accompanied with the production of Nabs. At day 14 after the first injection of vaccine 6/8 animals presented Nabs at low titers and at day 30 all animals were positive for the presence of Nabs. The second shot of vaccine allowed the increase of the neutralizing titer to 100 in all animals. We thus confirmed that the MOPEVAC_{MACV} was efficient to promote an Ab response.

### Efficiency of the MOPEVAC_{MACV} to Protect NHP

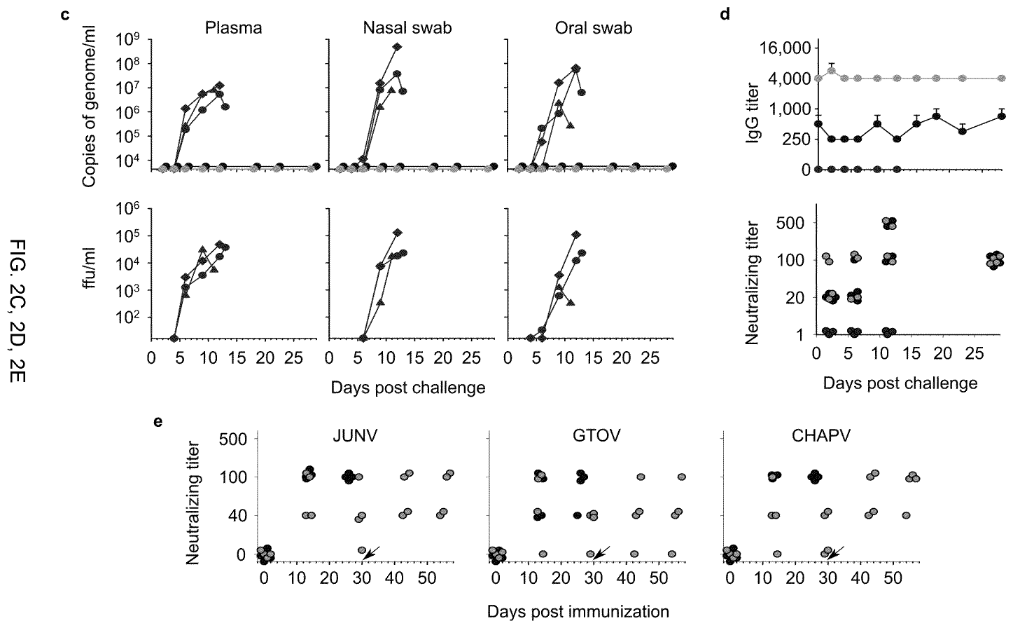

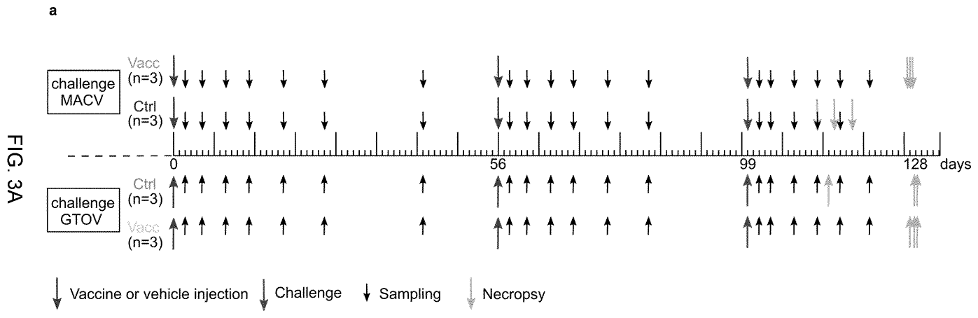

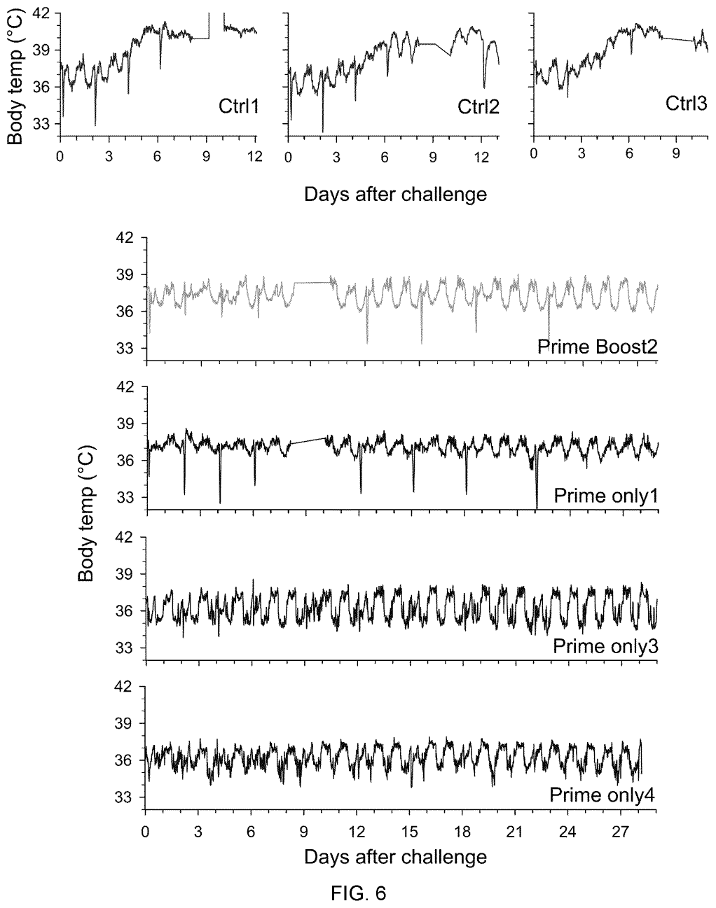

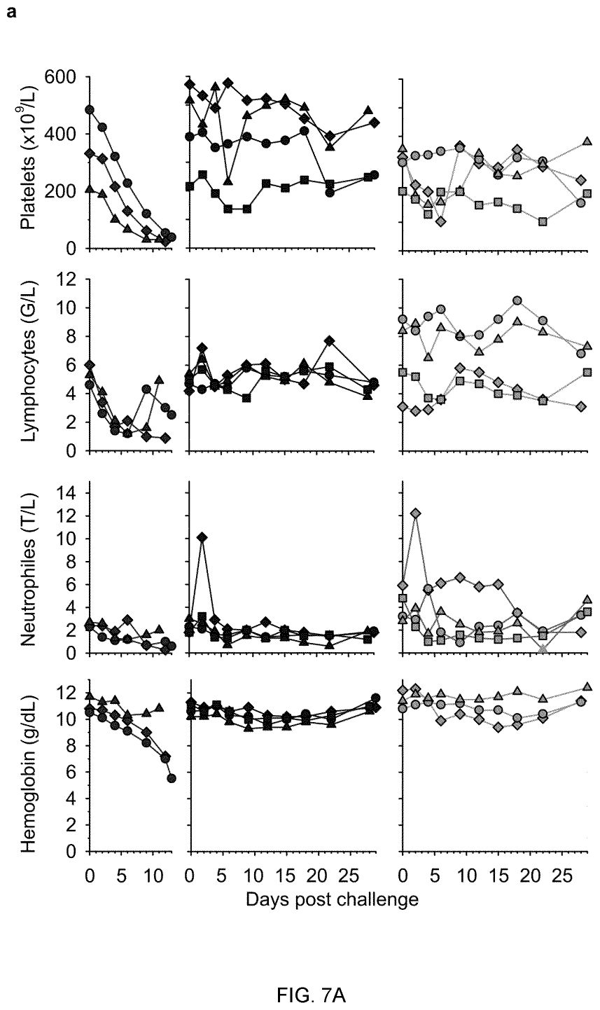

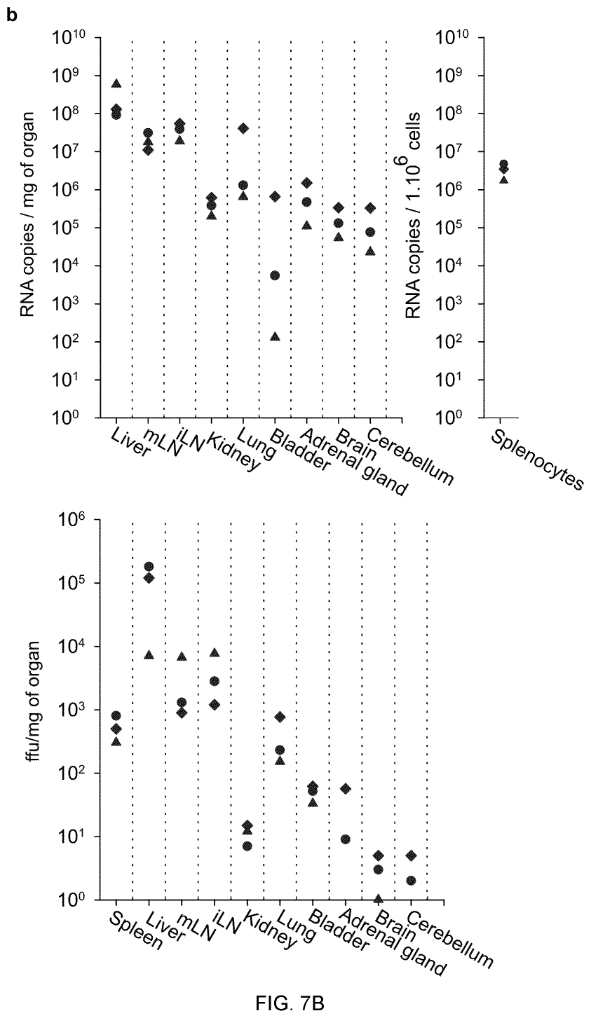

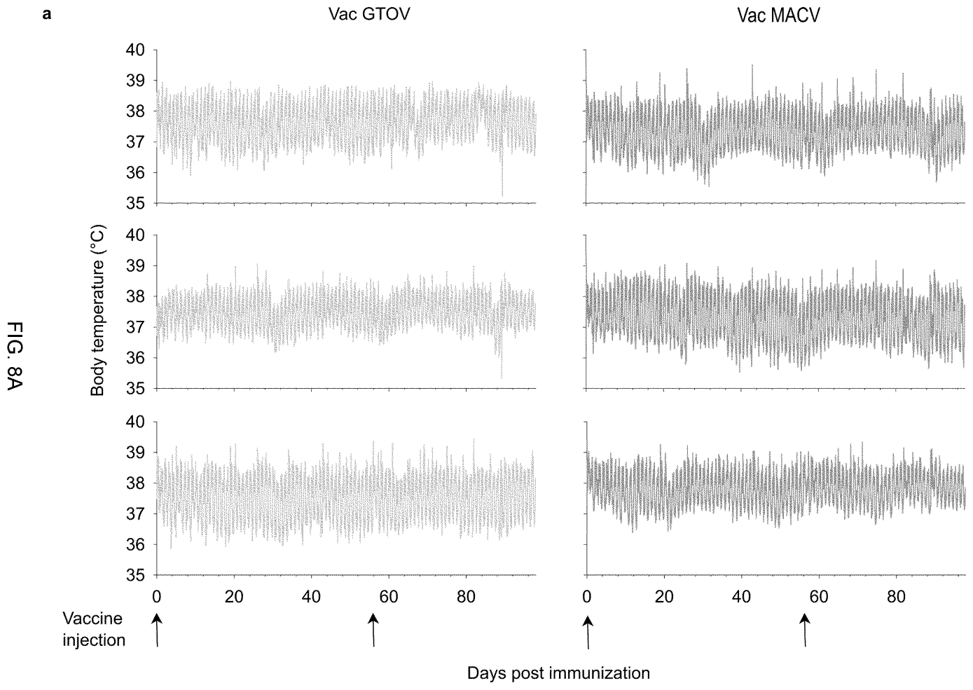

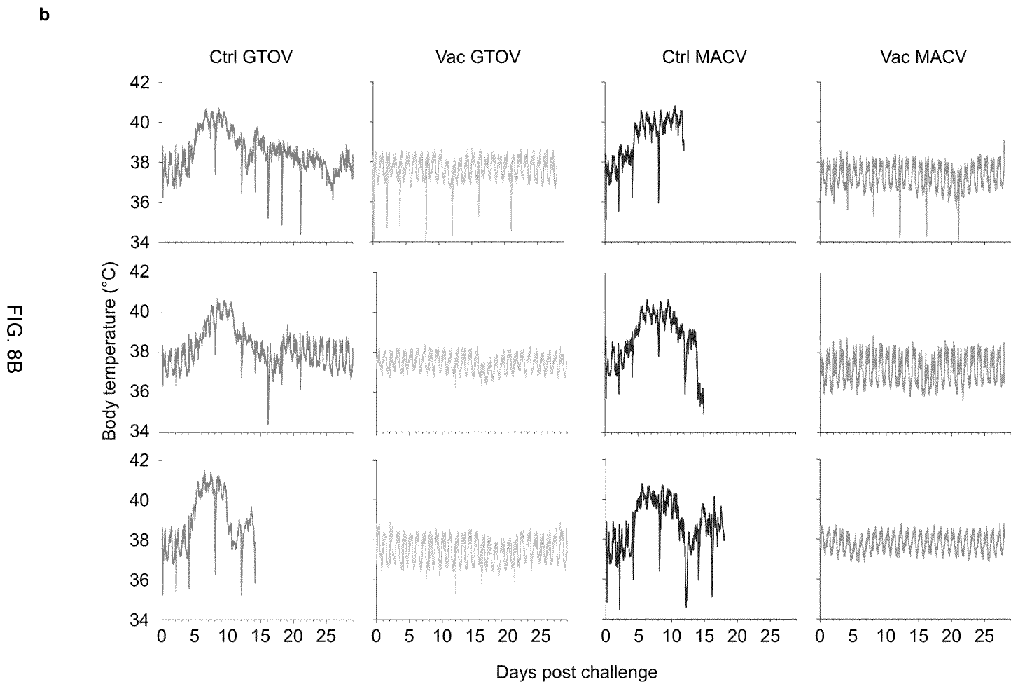

All animals received 3,000 ffu of MACV at the end of the immunization protocol. Each day, a clinical score was calculated depending on their behavior and reactivity, the loss of weight, the dehydration, the clinical and hemorrhagic signs and the rectal body temperature. This score remained nearby 0 during the whole protocol in vaccinated animals with no difference between the groups prime only or prime/boost immunization. Unvaccinated control animals experienced fever from day 4-5 (FIG. 6) and at day 8 they presented balance disorders, dehydration, and attenuated relationship with experimenters. They finally reached the ethical endpoint of the protocol between days 11 and 13 because of absence of reactivity, intense dehydration, and epistaxis. These animals continuously loosed weight from day 4 until the end of the protocol whereas the vaccinated ones did not evolve (FIG. 2a). Hematological evaluation did not highlight any disorder in vaccinated animals while all controls experienced deep lymphopenia and thrombocytopenia. A loss in hemoglobin concentration was also observed in 2/3 animals (FIG. 7a). We also looked at the modification of biochemical parameters, with a particular attention to eventual signs of hepatic or renal dysfunction that occur in hemorrhagic fever infections. Vaccinated animals did not present any modification of the parameters tested while the control ones had increasing levels of alanine aminotransferase (ALT), aspartate aminotransferase (AST), and plasmatic urea (FIG. 2b). They also presented a loss in albumin concentration. This indicates damage in hepatic function but also eventual renal injuries, rhabdomyolysis or dehydration.

These altered parameters were associated with a sustained virus replication. We were able to measure the presence of MACV in samples from day 6 in all plasma samples, including infectious virus. The presence of the virus in oral and nasal swabs was found in all control animals at day 9 and the titers obtained were considerably high considering the low quantity loaded on the swab and then diluted in conservation medium (FIG. 2c). The virus concentration raised until death. A slight drop just occurred for some samples in the very last days before the endpoint. For vaccinated animals, we were not able to detect viral RNA or infectious particles in any of the samples tested.

We also looked at the presence of virus in the organs at the day of euthanasia and we found that in controls the infection was pantropic. Each organ tested was positive in RT-qPCR. Infectious virus was also quantified except for the brain of one animal (FIG. 7b). For the vaccinated NHP, we did not detect any viral RNA in organs, suggesting that no virus persisted. Thus, the vaccination was able to offer a sterilizing immunity to NHP, even with a single dose.

We also looked at the antibody response after challenge. IgG specific of the virus were assayed. The vaccinated animals did not respond to the challenge by a boost of IgG synthesis. The titers remained quite stable all along the protocol. However, the Nabs raised around day 14. Plasma samples were able to neutralize the virus at a dilution between 1/100 and 1/500 (FIG. 2d). At the end of the protocol the titer returned to 100 for all vaccinated animals. We did not observe any heterogeneity in the Ab response depending on the vaccination protocol. In control animals, we did not detect neither specific IgG nor NAbs. The immune response elicited by MOPEVAC_{MACV} was able to protect NHP against a lethal challenge and offered a sterilizing immunity. We were interested to look at broad spectrum Nabs that could offer cross protection for other NW arenavirus infection. We thus assayed plasma samples from the immunization period with MOPEVAC_{JUNV,GTOV, CHAPV}. While all plasma samples presented detectable Nabs at day 26 when assayed with MACV and were able to neutralize at a 1/100 dilution at the end of the prime/boost protocol, they were less neutralizing when assayed with GPC from other viruses (FIG. 2e). Interestingly, the neutralization capability was better for JUNV and CHAPV than for GTOV that is the most phylogenetically distant⁶.

### MOPEVAC_{NEW} Conception and Immunization

Due to the low incidence of most of the pathogenic NW arenaviruses and to counteract the risk of emergence, we constructed MOPEVAC_{NEW}, a pentavalent vaccine. Five MOPEVAC viruses were included, each expressing a different GPC gene: MACV, GTOV, CHAPV, SABV and JUNV GPC. Our goal was to protect against all South American pathogenic arenaviruses and to benefit from a broad protection to protect against possible emerging new virus.

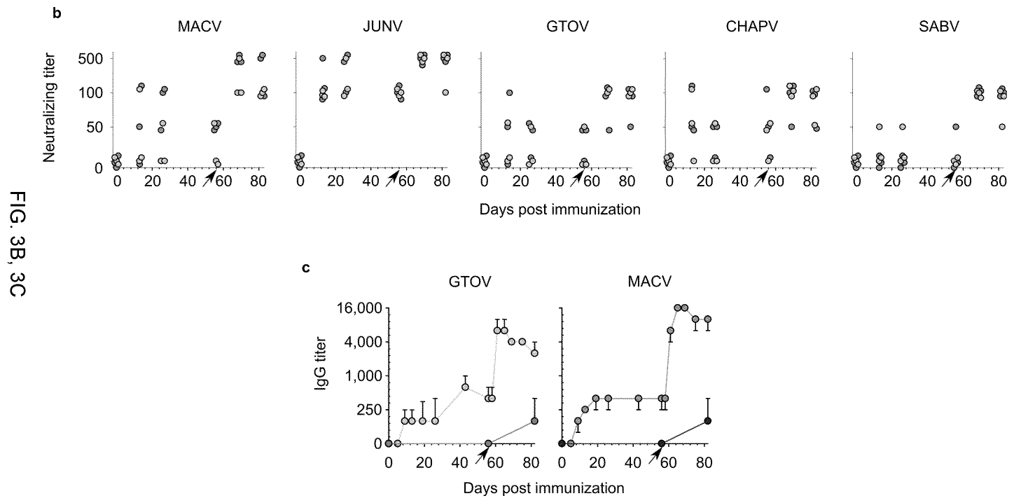

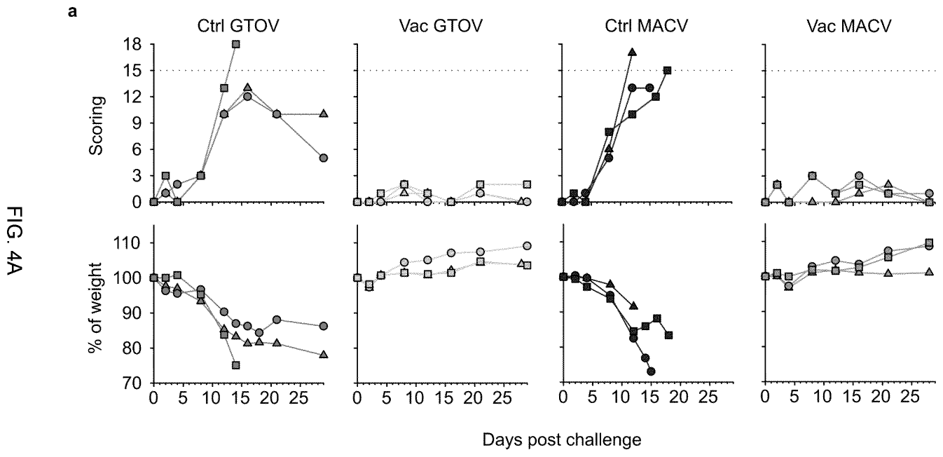

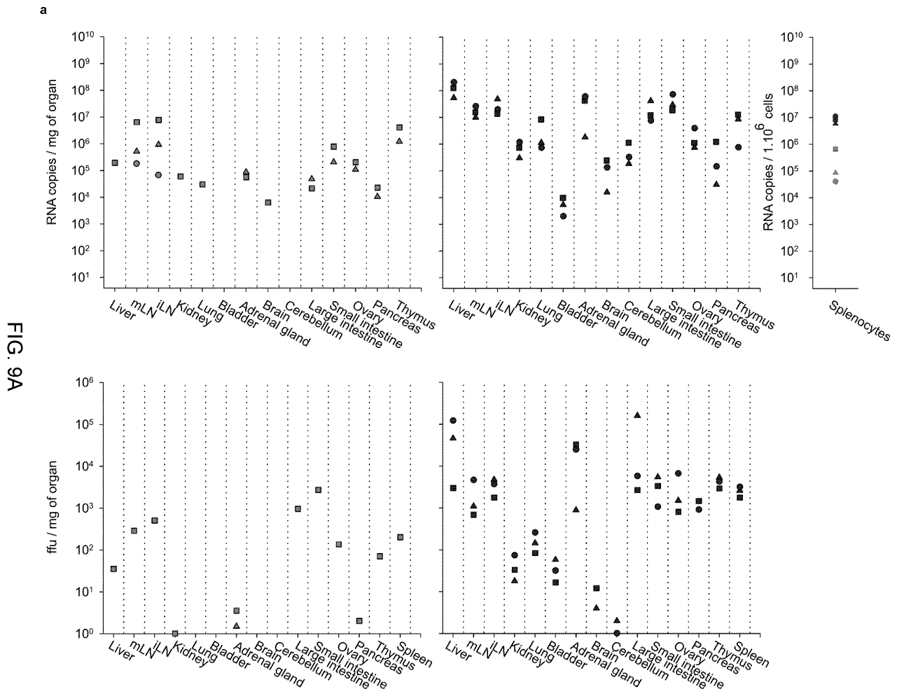

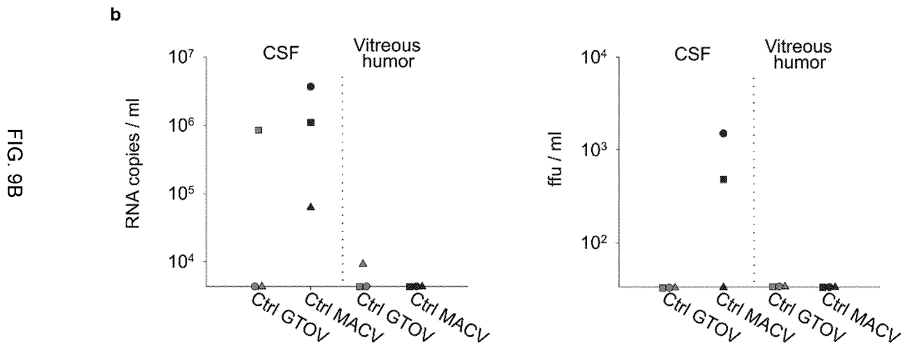

The previous experiment demonstrated the interest of Nabs to offer a sterilizing immunity and the benefit of the prime boost protocol to increase the titers of broadly neutralizing antibodies. We thus vaccinated 6 cynomolgus monkeys with 2.10⁶ ffu of MOPEVAC_{NEW} in a prime boost protocol, with equal doses of each valence. To optimize the immune response, we extended the prime period to 2 months and challenged the animals 1 month after boost (FIG. 3a). 6 animals received only the vehicle at the same time points and will be used as unvaccinated animals. We did not observe any clinical sign during the immunization period and no modification of the body temperature all along the protocol (FIG. 8a). We also looked at the shedding of the vaccine and we were not able to detect any virus in plasma samples, nasal or oral swabs, and urine in the days following injections. The vaccine is thus safe in macaques. We tested plasma samples for their ability to neutralize the different arenaviruses. We observed heterogeneous intensities of response to the immunization (FIG. 3b). We were not able to detect Nabs in all animals at the end of the prime period. However, the cut-off of detection was 2.5 times higher than in first experiment and could explain the lack of early detection of Nabs. The response to JUNV was stronger than to all other viruses with Nab titers that raised to at least 100 as soon as day 13 and reached 500 after boost. Other viruses all allowed the synthesis of Nabs that were eventually detectable after boost in all animals and raised a mean titer of 100.

We tested the IgG specific of the viruses for MACV and GTOV. We observed that, just like the previous experiment, vaccinated animals synthesized IgG in response to the vaccine and that the boost injection allowed a rapid and strong increase in the IgG titers. We also checked for the absence of unspecific response in control animals but we unfortunately measured low Ab levels at the end of the immunization protocol for one animal of each control group. This unspecific result is still not explicated.

### Challenge of NHP Vaccinated or Not with MOPEVAC_{NEW}

Animals were challenged with either MACV or GTOV, 3 vaccinated and 3 control animals each. Their clinical state was evaluated each day, as done during the first experiment. MACV and GTOV infection induced illness in all control animals. The evolution of the disease in MACV infected animals was closely related to the clinical observations of the first experiment. Animals reached the ethical endpoint at days 12, 15 and 18. Despite a clinical score of 13, one of them was euthanized because of a dramatic weight loss (27%). The GTOV infected NHP presented symptoms with two days of delay in comparison with MACV but all the control animals experienced illness with a loss of activity, gastrointestinal symptoms and fever (FIG. 8b). Only one reached the clinical score of 15 and was euthanized at day 14. The two remaining NHP had a clinical score of 13 and 12 respectively at day 15. This score then gradually decreased until the end of the protocol. One of them was less healthy at day 29 with a score of 10 versus 5 and a weight loss of 22% versus 14%. This animal could in fact had been considered at the ethical endpoint at this time (FIG. 4a). The vaccinated NHP did not experience fever or clinical signs whatever the virus used for infection. The low score attributed sometimes was due to diarrhea that was also observed for some animals before the challenge.

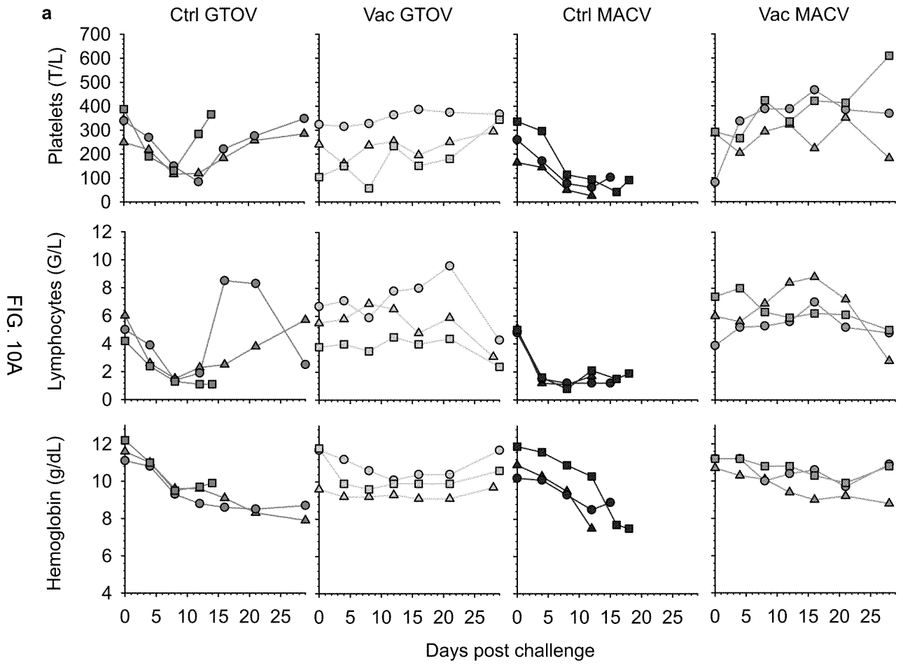

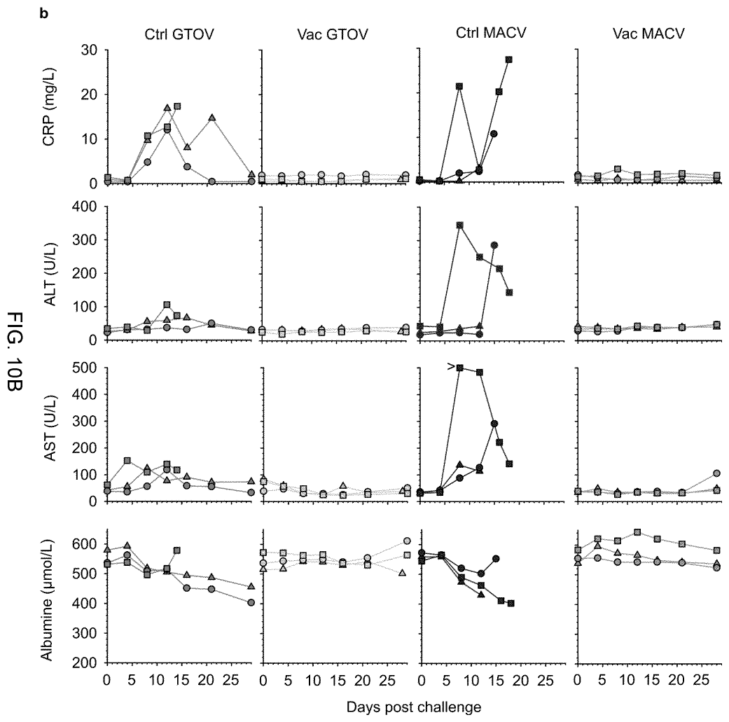

We looked for the presence of the virus at each sampling time. All control animals were found positive for viral RNA and for infectious particles (FIG. 4b). RNA was detected from day 8 in most of the samples. The RNA and infectious titers measured for MACV controls were very similar to those observed in the first experiment. The NHP infected with GTOV that was euthanized at day 14 had RNA concentrations equivalent to those of MACV infected animals. However, the infectious titers in plasma was around tenfold lower during the whole protocol. We also found infectious particles in the nasal samples from day 8 but infectious titer in oral swab was measured just at the detection limit and at day 12 only. The RNA concentration in oral swab at day 14 was nevertheless of 10⁸ copies of genome/ml. The two animals that survived until the end of the procedure did not control virus replication in a similar manner. The one who was the healthiest did not present infectious particles in oral swabs at any time and the virus disappeared from day 12 and 16 in plasma and nasal swabs respectively. The detection of genomic RNA persisted but at quite low titers. The other animal that survived presented elevated levels of viral RNA in the nasal swabs until the end of the protocol. At day 29, we also measured the presence of the viral genome in plasma and oral swab at low titers. We looked for the presence of the virus in the organs. Animals that were euthanized at the peak of the illness were all positive in RT-qPCR in all the organs tested except for the bladder and the cerebellum in GTOV infected animal. The quantity of genome detected was lower in this animal than in MACV infected ones. The pan tropism was not demonstrated like for MACV infection. We did not find infectious particles in all the organs tested, the secondary lymphoid organs, the intestine, the liver, and the ovaries were the most impacted but infectious titers remained lower than for MACV infection (FIG. 9a). All samples from vaccinated animals were also tested and we did not find any viral RNA in any of these samples, attesting that the vaccine offered the sterilizing immunity expected. We also looked at the presence of virus in immune preserved compartments: cerebrospinal fluid (CSF) and vitreous humor (FIG. 9b). We were able to detect viral genome in all LCR samples from animals at the peak of the disease but only two MACV infected animals had infectious particles. The samples from the control GTOV animals at day 29 and from all vaccinated NHP were negative. However, this indicates that the virus crossed the blood brain barrier in acute disease. The vitreous humor was negative for infectious particles in all the samples but one surviving GTOV infected control had viral RNA at low titers in the sample. Interestingly, this animal was the one that did not recover well from the illness.

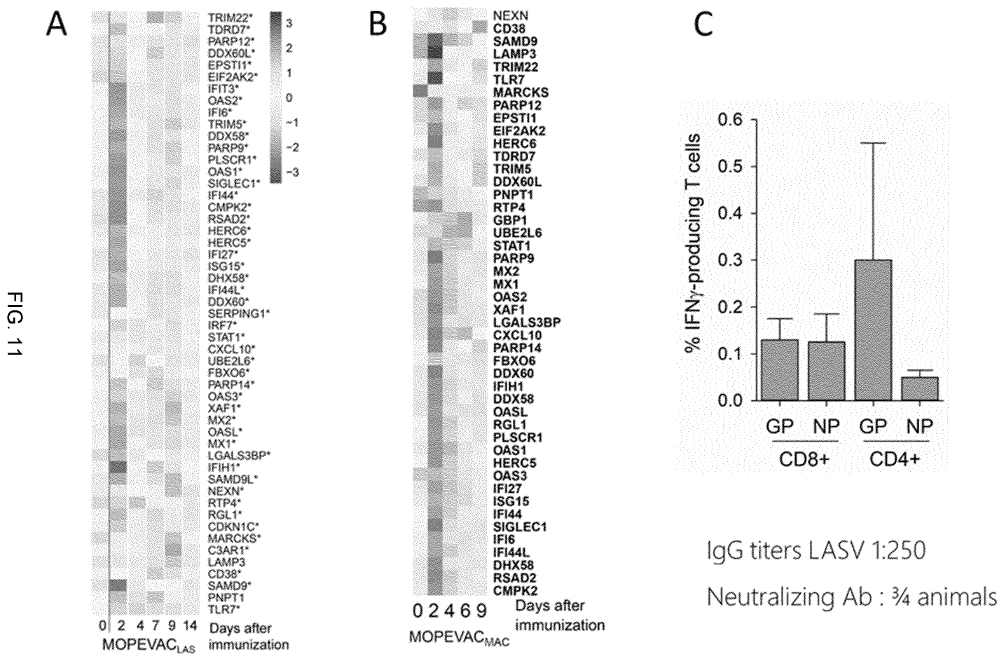

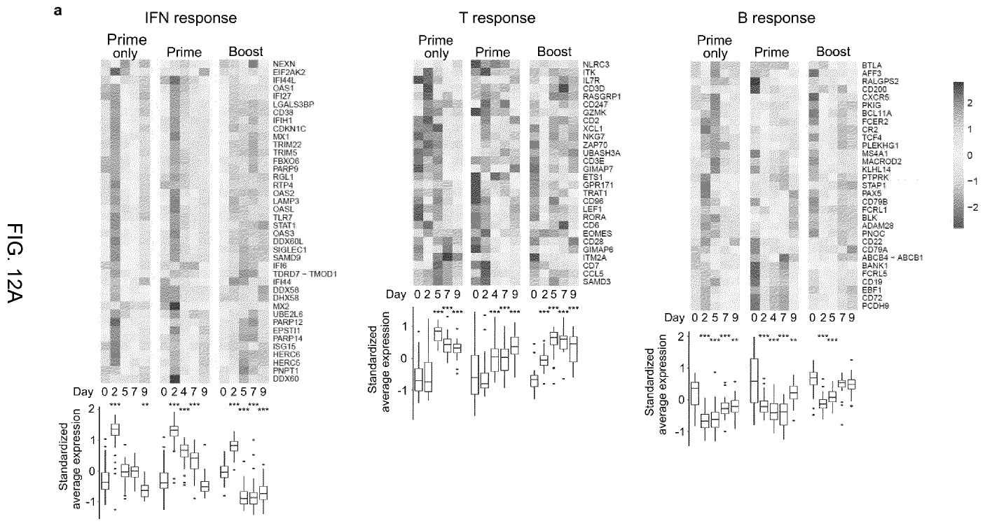

We were interested in the hematological and biochemical parameters during the course of the experiment. The vaccinated animals did not present any significant modification of the blood formula and of the markers of inflammation, hepatic and renal function (FIG. 10 a and b respectively). However, we observed lymphopenia, thrombocytopenia and loss of hemoglobin concentrations in all the control animals. This was more acute in MACV infected than in GTOV infected NHP. The animals that survived from the infection until the end of the protocol recovered from thrombocytopenia but they did not come back to normal concentrations of hemoglobin. One animal had a transient leukocytosis while the other one returned progressively to normal values of lymphocytes count. In a consistent matter, the biochemical parameters were more dysregulated in MACV controls. However, in both groups we observed inflammation with the increase of c-reactive protein (CRP), and a loss of albumin. The elevation of ALT and AST that reflects hepatic damage was restrained in GTOV infected animals but sustained in MACV infected ones.

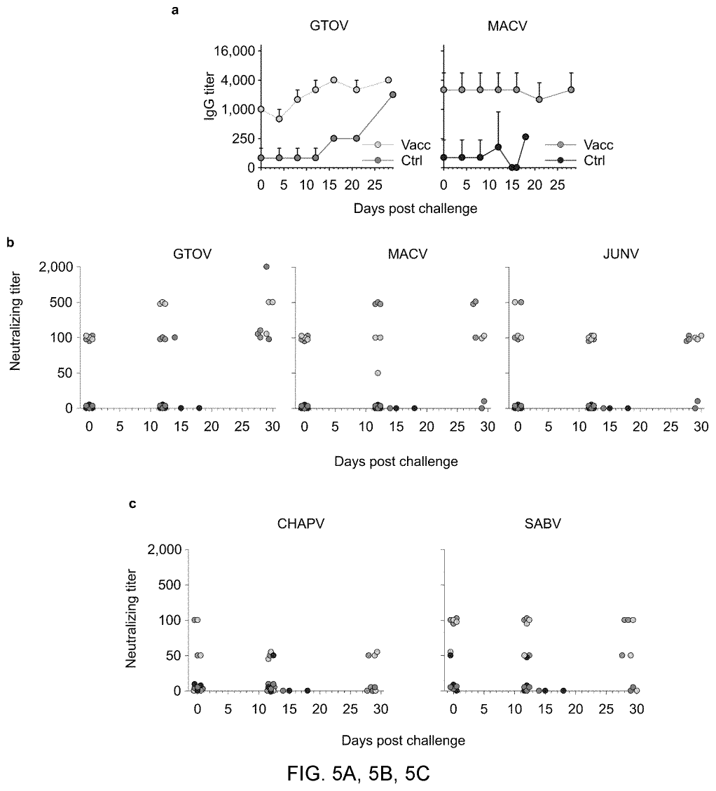

We did not observe antibody response in control animals until day 12. The IgG specific of the viruses remained at the same level during the whole protocol for MACV infected NHP, except for the one that was euthanized at day 18 that presented increased level of antibodies at the endpoint of the experiment (FIG. 5a). The GTOV infected controls that survived presented increasing antibody levels from day 16 with a heterogeneous kinetic as demonstrated by the error bars. In vaccinated animals, we did not observe any boost in the IgG levels for MACV, consistently with the first experiment, but the GTOV infected animals presented an increase in IgG concentrations to finally obtain the same IgG titers as in MACV NHP. Neutralization experiments were performed with wild type virus for GTOV, MACV and JUNV. The neutralizing titers observed at the day of the challenge were consistent with those obtained at the end of the immunization period. Interestingly, we could observe that the vaccinated NHP boosted the production of Nabs and that the neutralization titers were increased specifically for the virus used for infection (FIG. 5b). We did not observe any Nabs response in MACV infected controls but all GTOV infected ones presented significant levels of Nabs. In one animal that survived the titer raised to 2,000, more than in vaccinated animals. For CHAPV and SABV, we used MOPEVAC viruses to perform the neutralization experiment (FIG. 5c). While we observed a diminution in Nabs titers between the end of the immunization period and the challenge, vaccinated animals still presented detectable Nabs titers, except for two animals with MOPEVAC_{CHAPV}. Moreover, the Nabs titers tend to diminish slightly during the experiment period. One of the control animal infected with MACV present an unspecific low Nab titer in this experiment.

### Different Immune Responses Depending on the GPC Gene Expressed by MOPEVAC

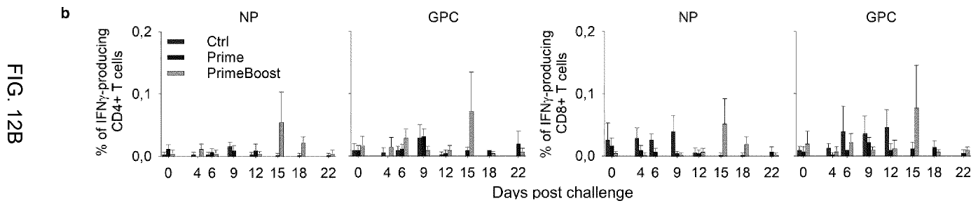

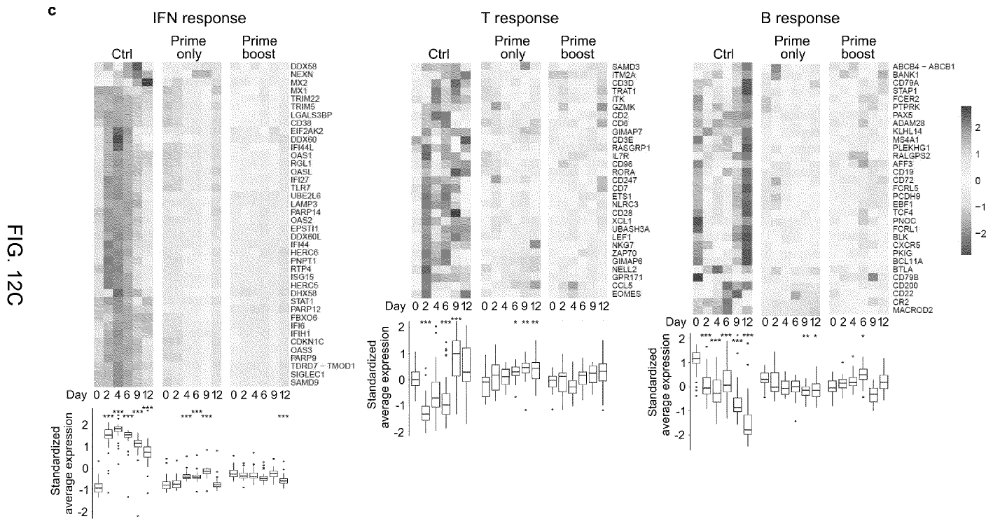

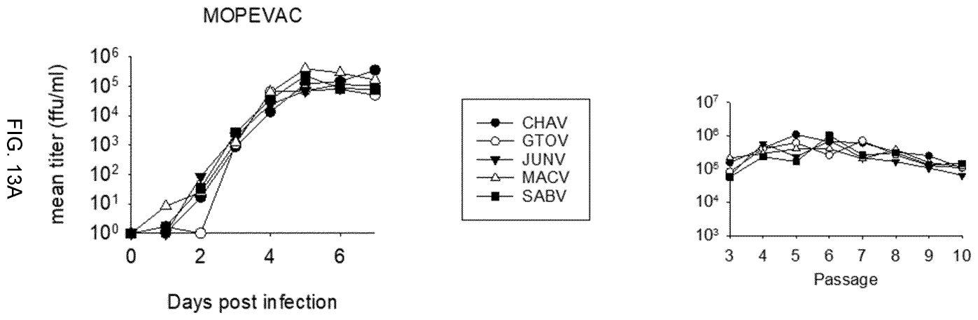

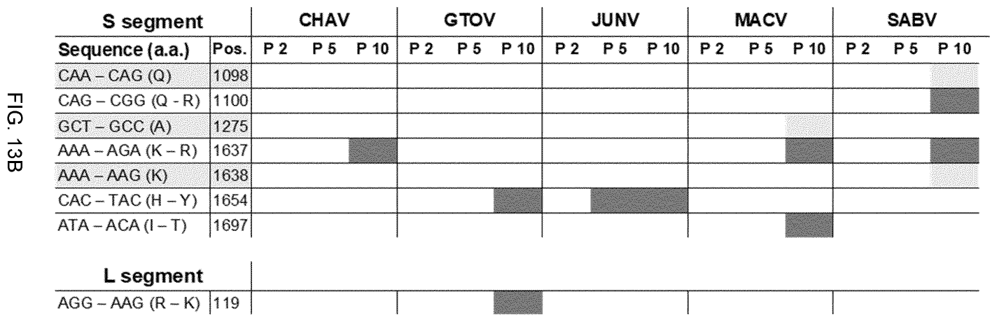

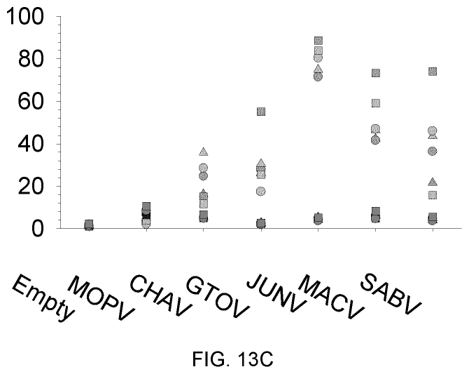

Transcriptomic analyses performed on PBMC after vaccine injection of either MOPEVAC_{LASV} or MOPEVAC_{MACV} resulted in the induction of a strong innate immune response in the very first days after immunization (FIG. 11A, B).

Interestingly, the adaptive immune response described above was not the same as the one observed after a MOPEVAC_{LASV} vaccine injection. Indeed, the immunization with MOPEVAC_{LASV} induced a Th1 cellular response associated with a cytotoxic phenotype in response to GPC and NP antigens. In contrast, we were not able to detect any Th1 T-cell response after MOPEVAC_{MACV} or MOPEVAC_{NEW} vaccination (FIG. 10C).

Moreover, we obtained higher IgG antibody titers after a MOPEVAC_{MACV} immunization than after a MOPEVAC_{LASV} immunization (FIG. 1C). Three over four monkeys had low titers neutralizing antibodies after the LASV vaccine, while all MACV vaccinated monkeys produced neutralizing antibodies at higher titers (1). The protocol and vaccine inoculum used were fully comparable between the two experiments. The differential orientation of the immune response was also reported in the literature (3). These results indicate that a similar vaccine vector, MOPEVAC, induces strikingly different immune responses, according to the GPC contained in the vector.

Actually, the differences in the immune responses observed after MOPEVAC_{LASV} immunization and immunization with New World arenaviruses MOPEVAC constructions clearly illustrate that MOPEVAC_{LASV} induces a cytotoxic T cellular response but with IgG neutralizing antibodies with lower titers than with MOPEVAC_{NEW} immunization. MOPEVAC_{NEW} induces IgG neutralizing antibodies at high levels, higher levels than with cytotoxic T cellular response but with IgG neutralizing antibodies with lower titers than MOPEVAC_{LASV}, but no detectable cytotoxic T cellular response. Therefore, the GPC introduced in the platform vector modifies the orientation of the immune response between Old World GPC/arenaviruses and New World GPC/arenaviruses. Of note, the entry receptor into host cells is different between Old World and New World arenaviruses. Specifically, MOPEVAC_{LAS} virus uses the alphadystroglycan receptor to enter inside the cells it infects (such as LASV does), and New World Arenaviruses use the transferrin receptor in order to enter inside the cells they infect, such as the MACV/JUNV/GTOV/SABV and CHAV MOPEVAC constructs described herein do. Therefore, the constructions described herein use the natural entry receptor of the targeted virus, i.e, the natural entry receptor of New World Arenaviruses. The different orientation of the immune responses between Old World GPC/arenaviruses and New World GPC/arenaviruses shown herein was not straightforward and comes out as a surprise.

### Ongoing Experiments

MOPEVAC_{NEW} has been tested and was efficient to protect against Machupo and Guanarito viruses infections. To complete the preclinical evaluation of MOPEVAC_{NEW} vaccine, further studies are planned. Non-human primate experiments including a total of up to 24 animals will be done to test the efficiency of MOPEVAC_{NEW} to protect against Junin, Sabia, and Chapare viruses that are targeted by the vaccine. Inventors will also experiment the capacity to cross protect: non-human primates will be infected with another virus, such as the Whitewater Arroyo virus, i.e., a closely related arenavirus classified in clade D of New World arenaviruses, in particular a virus not included in the vaccine formulation. These experiments will further assess the efficiency of MOPEVAC_{NEW} against all the viruses targeted and the induced cross protection against other closely related emerging viruses. The experimental design will be the same than the one used in the experiment that validated the vaccine against a challenge with Machupo and Guanarito viruses.

It is also planned to test the long lasting immune memory after vaccination but also the minimum delay to protect against a lethal infection. Animals will be vaccinated with MOPEVAC_{NEW} prior to infection, in particular up to one year before infection, with one of the viruses targeted by the vaccine. A vaccination with one injection and a vaccination with two injections will be tested to compare the efficiency between these two immunization strategies. Another group of animals will be vaccinated in the very last days before challenge, about eight days, to test the efficiency of the vaccine for a quickly induced protection. These results will assess the potency of the vaccine to rapidly protect population in case of emergence of the virus. Production of antibodies, in particular neutralizing antibodies, will also be further analyzed, and the immune responses will be characterized by measurement of soluble factors as well as the presence of the virus used for challenge in the fluids and in the organs of the studied animals.

### Discussion

The animal experiments presented herein provide evidence that the MOPEVAC platform previously assayed in the art for Old World arenaviruses can be used in a multivalent way against New World arenaviruses.

While in the art the use of the MOPEVAC platform, based on the MOPV virus, as a safe vaccine for vaccination against LASV has been shown, both LASV and MOPV pertain to Old World arenaviruses and MOPV is known as a natural vaccine against LASV infection. In present invention, the MOPEVAC platform has been used for vaccination against very divergent viruses where no incent towards the possibility of achieving a protection has ever been reported. It was therefore not straightforward that a protection could be achieved. Furthermore and outstandingly, due to the production of neutralizing antibodies at high titers, a sterilizing immunity that was not achieved against LASV, was obtained.

Given that the plasmas of the immunized animals could neutralize all tested New World arenaviruses, the vaccine injected was fully efficient to protect in vivo against the two tested New World arenaviruses, which are the MACV and GTOV viruses. These viruses are distant viruses: therefore, as the inventors provided evidence of full protection against two distant New World arenaviruses, the devised vaccine could probably protect efficiently against all viruses included in the vaccine. One can also look for the protection from a virus that is not included in the vaccine since protection against different, divergent, New World arenaviruses was obtained. Evidence is in particular provided that the vaccine injected would probably be efficient against New World arenaviruses pertaining to Clade B, but this evidence readily extends to New World arenaviruses pertaining to Clade D (i.e., viruses resulting from a recombination between Clades A and B). Literature describing phylogenetic distances in arenaviruses encompasses Gonzalez JP, Emonet S, Lamballerie X de, Charrel R. Arenaviruses. In: Current topics in microbiology and immunology [Internet]. 2007. p. 253-88. Available from: http://link.springer.com/10.1007/978-3-540-70962-6_11 (13). The distance between tested viruses makes it credible that efficiency of the vaccine against other pathogenic New World arenaviruses, especially all other pathogenic New World arenaviruses can be achieved. These pathogenic New World arenaviruses share the same entry receptor with the tested arenaviruses.

Strinkingly, the inventors did not observe any deleterious effect like antibody-dependant enhancement that could eventually occur because of an inefficient neutralization of short-proximity viruses. On the contrary, the inventors were able to measure equally efficient neutralization against all the viruses tested: these neutralizing antibodies seem to be the key of the protection. Antibody-dependant enhancement is for example described in Rey FA, Stiasny K, Vaney M-C, Dellarole M, Heinz FX. The bright and the dark side of human antibody responses to flaviviruses: lessons for vaccine design. EMBO Rep. 2018 February; 19 (2): 206-24 (14), and was a real problem to be concerned with in the context of a multivalent composition vaccine.

Thus, the invention paves the way to a vaccine able to protect against all New World arenaviruses even those that could have not still emerged. The distance of the tested New World virus from the prototypic Mopeia virus does not alter the efficiency of the vaccine. The orientation of the immune response after vaccination is different depending on the glycoproteins carried by the vector and reproduces the natural immunity observed after infection with the different viruses: cellular response for LASV (Old World arenavirus) and humoral response for New World arenaviruses. The cross-neutralization achieved, notably without any deleterious effect, especially without destructive cross-reactivity, between the different vaccine constructs described herein could strengthen the efficiency of the resulting pentavalent vaccine and offer a protection against new emerging arenaviruses.

This would be highly beneficial with regards to the requirements for development and production of new vaccines. Indeed, whereas interest at the moment to provide prophylaxis against those pathogens may be less obvious except for JUNV that is endemic the recent emergence of huge epidemics have highlighted the importance for preparedness against viruses with high potential to become epidemic, and arenaviruses are considered to be at risk of emergence.

### REFERENCES

1. Mateo, M. et al. Vaccines inducing immunity to Lassa virus glycoprotein and nucleoprotein protect macaques after a single shot. Sci. Transl. Med. 11, (2019).
2. Enria, D. A., Briggiler, A. M., Fernandez, N. J., Levis, S. C. & Maiztegui, J. I. Importance of dose of neutralising antibodies in treatment of Argentine hemorrhagic fever with immune plasma. Lancet (London, England) 2, 255-256 (1984).
3. Mantlo, Paessler & Huang. Differential Immune Responses to Hemorrhagic Fever-Causing Arenaviruses. Vaccines 7, 138 (2019).
4. Maiztegui, J. I., Fernandez, N. J. & de Damilano, A. J. Efficacy of immune plasma in treatment of Argentine haemorrhagic fever and association between treatment and a late neurological syndrome. Lancet (London, England) 2, 1216-1217 (1979).
5. Carnec, X. et al. A Vaccine Platform against Arenaviruses Based on a Recombinant Hyperattenuated Mopeia Virus Expressing Heterologous Glycoproteins. J. Virol. 92, (2018).
6. Nunberg, J. H. & York, J. The curious case of arenavirus entry, and its inhibition. Viruses 4, 83-101 (2012).
7. Urata S, Yasuda J. Molecular Mechanism of Arenavirus Assembly and Budding. Vol. 4, Viruses. 2012.
8. Zong M, Fofana I, Choe H. Human and host species transferrin receptor 1 use by North American arenaviruses. J Virol [Internet]. 2014 Jun. 11. 2014 August; 88(16): 9418-28. Available from: https://pubmed.ncbi.nlm.nih.gov/24920811
9. Li K, Lin X-D, Wang W, Shi M, Guo W-P, Zhang X-H, et al. Isolation and characterization of a novel arenavirus harbored by Rodents and Shrews in Zhejiang province, China. Virology [Internet]. 2015; 476:37-42. Available from: https://www.sciencedirect.com/science/article/pii/S0042682214005340
10. Kenyon RH, Green DE, Eddy GA, Peters CJ. Treatment of junin virus-infected guinea pigs with immune serum: development of late neurological disease. J Med Virol. 1986 November; 20(3): 207-18.
11. Sarute N, Ross SR. New World Arenavirus Biology. Annu Rev Virol. 2017 September; 4(1): 141-58.
12. South American Hemorrhagic Fevers: A summary for clinicians deFrank, 2021, International journal of infectious diseases, https://doi.org/10.1016/j.ijid.2021.02.046
13. Gonzalez JP, Emonet S, Lamballerie X de, Charrel R. Arenaviruses. In: Current topics in microbiology and immunology [Internet]. 2007. p. 253-88. Available from: http://link.springer.com/10.1007/978-3-540-70962-6_11
14. Rey FA, Stiasny K, Vaney M-C, Dellarole M, Heinz FX. The bright and the dark side of human antibody responses to flaviviruses: lessons for vaccine design. EMBO Rep. 2018 February; 19(2):206-24

1-18. (canceled)

19. A multivalent immunogenic composition comprising recombinant live attenuated Mopeia viruses (MOPV), wherein each valence is constituted by a recombinant live attenuated Mopeia virus wherein the expressed nucleoprotein (NP) and glycoprotein precursor (GPC) are encoded by the viral genome wherein:
a. the nucleic acid of the S segment encodes MOPV nucleoprotein (NP) having attenuated exonuclease activity, and
b. the nucleic acid of the S segment is deleted for the ORF of the glycoprotein precursor (GPC) of the Mopeia virus and comprises a heterologous nucleic acid encoding a New World arenavirus glycoprotein precursor (GPC) from one of the following arenaviruses: Machupo virus (MACV), Sabia virus (SABV), Chapare virus (CHAPV), Junin virus (JUNV) and Guanarito virus (GTOV).

20. The multivalent immunogenic composition according to claim 19, wherein:
a. the MOPV nucleoprotein (NP) comprises amino acid substitutions at positions D390, H430 and D467 with respect to SEQ ID NO: 1, or
b. the MOPV nucleoprotein (NP) comprises amino acid substitutions at positions D390, H430 and D467 with respect to SEQ ID NO: 1, and at least one further amino acid substitution at a position selected from E392, G393, H529, and D534 with respect to SEQ ID NO: 1.

21. The multivalent immunogenic composition according to claim 20, wherein:
a. the MOPV nucleoprotein (NP) comprises amino acid substitutions at positions D390, H430 and D467 that are D390A, H430A and D467A substitutions with respect to SEQ ID NO: 1, or
b. the MOPV nucleoprotein (NP) comprises amino acid substitutions at positions D390, H430 and D467 that are D390A, H430A and D467A substitutions with respect to SEQ ID NO: 1, and at least one further amino acid substitution at a position selected from: E392, G393, H529, and D534 that is selected from: E392A, G393A, H529A, and D534A substitution(s) with respect to SEQ ID NO: 1.

22. The multivalent immunogenic composition according to claim 19, which further comprises at least one further recombinant live attenuated Mopeia virus wherein the expressed nucleoprotein (NP) and glycoprotein precursor (GPC) of said virus are encoded by the viral genome wherein:
a. the nucleic acid of the S segment encodes MOPV nucleoprotein (NP) having attenuated exonuclease activity, and
b. the nucleic acid of the S segment is deleted for the ORF of the glycoprotein precursor (GPC) of the Mopeia virus and comprises a heterologous nucleic acid encoding a New World arenavirus glycoprotein precursor (GPC) from one of the following arenaviruses: Amapari virus, Flexal virus, Latino virus, Oliveros virus, Parana virus, Patawa virus, Pichinde virus, Pirital virus, Tacaribe virus, Tamiami virus, and Whitewater Arroyo virus.

23. The multivalent immunogenic composition according to claim 19, wherein the amino acid sequence of the GPC is selected from the group of the sequences SEQ ID NO: 4 for the Machupo virus (MACV), SEQ ID NO: 5 for the Sabia virus (SABV), SEQ ID NO: 6 for the Chapare virus (CHAPV), SEQ ID NO: 7 for the Junin virus (JUNV) and SEQ ID NO: 8 for the Guanarito virus (GTOV), respectively.

24. The multivalent immunogenic composition according to claim 19, which is free of adjuvant(s) of the immune response and/or immunostimulant component(s).

25. The multivalent immunogenic composition according to claim 19, wherein the composition, comprises the five different recombinant live attenuated Mopeia viruses (MOPV).

26. The multivalent immunogenic composition according to claim 25, which is a pentavalent composition where valences comprise the five different recombinant live attenuated Mopeia viruses (MOPV) and all valences are present at an equal dose.

27. The multivalent immunogenic composition according to claim 19, which is dosed between 1.10² and 1.10¹² ffu (Focus-forming units), as measured by virus titration, the dose being given for the total of the cumulated valences of the different recombinant live attenuated Mopeia viruses (MOPV) which are present in the multivalent immunogenic composition.

28. A vaccine or therapeutically effective composition comprising the multivalent immunogenic composition according to claim 19 with any one of:
pharmaceutically acceptable carrier(s), delivery vehicle(s), excipient(s), preservative(s), or any combination thereof.

29. A method for eliciting a protective immune response in a mammalian host, against a New World arenavirus infection, or treating a mammalian host which has been infected with a New World arenavirus,
wherein the immunogenic composition of claim 25 is administered as a single composition or as separate active ingredients to a host in need thereof.

30. The method according to claim 29, wherein the protective immune response is a cellular and/or a humoral response against a New World arenavirus.

31. The method according to claim 29, wherein the elicited immune response is a prophylactic immune response against the New World arenavirus infection or New World arenavirus symptom or disease, or is a therapeutic immune response against the New World arenavirus infection or New World arenavirus symptom or disease.

32. The method according to claim 29, wherein neutralizing antibodies against a New World arenavirus are elicited.

33. The method according to claim 29, wherein administration achieves a cross-neutralization against another New World arenavirus.

34. The method according to claim 29, wherein administration is to a human individual in need thereof according to a prime immunization regimen or according to a prime-boost immunization regimen.

35. A method of preparing a recombinant live attenuated Mopeia virus (MOPV) in a eukaryotic host cell, said recombinant live attenuated Mopeia virus (MOPV) comprising an heterologous nucleic acid encoding a New World arenavirus GPC from an arenavirus selected among: Machupo virus (MACV), Sabia virus (SABV), Chapare virus (CHAPV), Junin virus (JUNV) and Guanarito virus (GTOV), wherein the method comprises the steps of:
transfecting the eukaryotic host cell with plasmids wherein:
a first plasmid that comprises a polynucleotide which is an expression cassette encoding the L segment antigenomic transcript of a Mopeia vRNA (L vRNA segment expression cassette);
a second plasmid that comprises a polynucleotide which is an expression cassette encoding a chimeric S segment antigenomic transcript of a Mopeia vRNA, in particular a S segment that is deleted for the ORF of the glycoprotein precursor (GPC) of the Mopeia virus, wherein the polynucleotide comprises (i) the ORF of the GPC protein of a New World arenavirus selected among: Machupo virus (MACV), Sabia virus (SABV), Chapare virus (CHAPV), Junin virus (JUNV) and Guanarito virus (GTOV), and (ii) the ORF of a nucleoprotein (NP) protein which is mutated by amino acid residue substitution(s) in the wild type NP of the Mopeia virus to have attenuated exonuclease activity;
an expression cassette for the L protein of the Mopeia virus wherein said cassette is either present as an insert in the second plasmid or is contained in a third plasmid;
an expression cassette for the NP protein of the Mopeia virus wherein said cassette is either as an insert in the first plasmid or is contained in fourth plasmid;
allowing ribonucleoproteins of the recombinant Mopeia virus to form and expression of the New world arenavirus GPC gene to assemble into recombinant live attenuated viral particles and;
recovering recombinant live attenuated Mopeia virus expressing the GPC of a New World arenavirus selected among: Machupo virus (MACV), Sabia virus (SABV), Chapare virus (CHAPV), Junin virus (JUNV) and Guanarito virus (GTOV).

36. A method of preparing a multivalent, immunogenic composition comprising recombinant live attenuated Mopeia viruses (MOPV) expressing a GPC protein of a New World arenavirus selected among: Machupo virus (MACV), Sabia virus (SABV), Chapare virus (CHAPV), Junin virus (JUNV) and Guanarito virus (GTOV), the method comprising the steps of:
a. preparing recombinant live attenuated Mopeia viruses (MOPV) according to the method of claim 35, and
b. associating the recovered recombinant live attenuated Mopeia viruses wherein each of the recombinant MOPV expresses a GPC of a New World arenavirus selected from the group of Machupo virus (MACV), Sabia virus (SABV), Chapare virus (CHAPV), Junin virus (JUNV) and Guanarito virus (GTOV to provide a multivalent immunogenic composition or vaccine wherein collectively all of said GPC are expressed and wherein the quantitative proportion of each valence of MOPV in the composition is identical.


SEQUENCE LISTING (21 sequences)

SEQ ID NO: 1 - protein, 570 aa, Mopeia virus
  REGION 1..570: note = MISC_FEATURE - WT nucleoprotein of the MOPV AEO89356.1 nucleoprotein [Mopeia mammarenavirus]
MSNSKEVKSFLWTQSLRRELSGYCSNIKIQVIKDAQALLHGLDFSEVANVQRLMRKEKRDDSDLKRLRDLNQAVNNLVELKSVQQKNVLRVGTLTSDDLLVLAADLDRLKAKVIRGERPLAAGVYMGNLTAQQLEQRRVLLQMVGMGGGFRAGNTLGDGIVRVWDVRNPELLNNQFGTMPSLTIACMCKQGQADLNDVIQSLSDLGLVYTAKYPNMSDLDKLSQTHPILGIIEPKKSAINISGYNFSLSAAVKAGACLIDGGNMLETIKVTKSNLEGILKAALKVKRSLGMFVSDTPGERNPYENLLYKLCLSGEGWPYIASRTSIVGRAWDNTTVDLSGDVQQNAKPDKGNSNRLAQAQGMPAGLTYSQTMELKDSMLQLDPNAKTWIDIEGRPEDPVEIAIYQPNNGQYIHFYREPTDIKQFKQDSKHSHGIDIQDLFSVQPGLTSAVIESLPKNMVLSCQGADDIRKLLDSQNRRDIKLIDVSMQKDDARKFEDKIWDEYKHLCRMHTGIVTQKKKRGGKEEVTPHCALLDCLMFEAAVIGSPQIPTPRPVLSRDLVFRTGPPRVVL

SEQ ID NO: 2 - DNA, 3334 bp, Mopeia virus
  misc_feature 1..3334: note = JN561684.1 Mopeia virus strain AN 21366-BNI segment S glycoprotein precursor (GPC) and nucleoprotein (NP) genes, complete cds
  source 1..3334: mol_type = unassigned DNA
ggagatggggcagatagtcaccttctttcaagaggtgccacacatccttgaagaagtgatgaacattgtgctgatgaccctctcaatcttggccatcctaaagggcatctacaatgtgatgacctgtggaatcatcggtttgataacatttttgttcttgtgtgggagatcatgctcaagcatctataaggacaactatgagttcttctctctcgacctcgacatgtcttcactgaatgcaacgatgcctctctcctgctcaaagaacaactcccatcactacatccaagttgggaatgagacaggcctagagctgacattgacaaacactagcataattgatcataagttttgcaacctgtctgatgctcacaggaggaatctttatgacaaagcacttatgtcaatcttgacaacattccacctaagcattccagattttaaccaatatgaggcaatgtcatgtgattttaacggagggaagatctcaattcaatacaacctgtcccactcaaattatgtggatgccggcaaccactgtggcactattgcaaatggcattatggatgtttttagaagaatgtattggagcacctccctttcggttgcctctgacataagtgggactcaatgcatacagaccgattataagtatttgattattcagaacacatcatgggaggatcattgcatgttctcaagaccctcacccatgggatttttaagccttctgtcacagagaaccaggaatttctacatctcaagaagacttttgggtctttttacatggactttgagtgactcggaagggaacgacatgccaggtggttactgtctcacaaggtctatgctaatagggcttgatctgaaatgctttgggaacactgccattgcgaagtgtaatcaggcacatgatgaagaattctgtgacatgctccgcctttttgattttaataaacaagcaattagcaaactgaggtctgaagttcagcagagcataaatttgataaataaagctgtcaacgccctcatcaatgatcaactggtcatgaggaaccatttgagagatctaatgggtattccttattgcaactactctaagttttggtacttgaatgatactaggacagggagaacatccctcccaaagtgttggcttgtgaccaatgggtcatacctaaatgaaacccagttctcaacagaaattgagcaagaagccaataacatgttcactgacatgttgaggaaggagtatgagaaaaggcagagcacaacacctctggggttagtagatctttttgttttctccactagcttttatttgatctccgtgttcctccacctaatcaaaattccaacacatagacacataaaaggcaagccttgcccgaaaccacacagactcaatcacatggcaatctgttcctgtggcttctataagcaaccaggtctccccacacaatggaaaaggtgaagaacaggggcacccccgagacccaccgccttcggcggtgggtctcgggggcctggagtgactgttccttgatggcctccacgacccaccgccagaggcggtgggtcgtggaggtgtcccttcttacaggacaactctgggaggacctgttctaaacaccaggtctctactcaagactggtctgggggttggaatttgtggactccctatgactgctgcttcaaacatgagacaatccagcaatgcacagtgtggtgtcacttcttctttgccacctctcttcttcttttgcgttacaatccccgtatgcattctacaaaggtgtttgtattcatcccagatcttatcctcaaattttcttgcatcgtctttctgcatggacacatcaatcagttttatgtccctcctgttctgggagtcaagaagctttctgatgtcatcagcaccttgacacgacaagaccatgttctttggcaggctctcaattacagcacttgtcaaccccggctgaactgagaataggtcttggatgtcaatgccatgagagtgtttggagtcttgtttgaattgtttaatgtctgttggttccctgtaaaaatgaatatactgaccattattaggttgatagatagctatctccacggggtcttcaggtctcccttctatgtcaatccatgtcttagcatttggatccaattgcaacatgctgtctttgagttccattgtctgagagtaggtcaaaccagcaggcatgccttgggcctgagcgagtctgttggagttacctttgtcaggctttgcattctgttgcacatcaccactcagatcaacagttgtgttatcccaagccctgccgacgatcgatgttcttgatgctatgtaaggccaaccctctccagaaagacatagtttgtagaggagattttcataagggttcctttcccctggcgtgtcagagacaaacattcccaaagaacgcttgactttcaaggcagccttcaaaattccttccaaattggattttgttactttgatggtctccagcatgtttccgccgtctattagacaagcaccagctttcaccgcagctgacaggctaaaattgtaccctgatatgtttatggcacttttcttgggctcaatgatccccaagattgggtgggtctgagagagtttgtctaagtcagacatgtttggatactttgcagtgtacacaagccccaagtctgacaacgattggatcacatcattcagatctgcctgcccttgtttgcacatgcaagcaatcgtcaggcttggcattgtcccaaactgattgtttaaaagctctgggtttcgaacatcccacactctaacaatgccatctccgagagtgtttcctgcccggaacccgccacccattccgaccatctgtaacaaaaccctcctctgttctagctgctgagctgttaggttgcccatatagactccagcagcaagaggcctctcacctctgatgacttttgctttgagtctgtccaggtcggcagcaaggacgaggaggtcatcagaggttagtgtccccactctcaaaacattcttctgttggactgactttaactcaactagattgttcactgcctggtttaggtccctcaatcttttcaggtcagagtcatccctcttctcctttctcatcaacctttgaacattggcaacttcagagaagtccagcccatgaagaagtgcttgagcatccttgatgacttggatctttatgttggagcagtaccctgagagttctctcctcaggctctgtgtccacaagaaggacttcacctcctttgaattggacattgttggaggcttttcggtgcagag

SEQ ID NO: 3 - DNA, 7271 bp, Mopeia virus
  misc_feature 1..7271: note = JN561685.1 Mopeia virus strain AN 21366-BNI segment L, complete sequence
  source 1..7271: mol_type = unassigned DNA
cgcaccggggatcctaggcaattttgtggatctttgctgatacttcattctgcactcaaagccgcatccggcaagagtcatggggaaaacgcagtccaagggacagcccagcacaaatctcctcaaggagacagaaccaaggcatccagtaataccagatgccaggggaactggccctgaattctgcaagagctgctggttcgaaaggagggggttagtcaggtgcaacgatcactacctatgtttgaactgtcttacacttttacacacagtgtcagataggtgccccatctgcaaacacaaactacccttcagactagagctccagacccagccgacagcaccaccagaaatcccaccatcccaaaatcccccaccctacagcccctgaggcagaactccgccccgaagcccctcccaaacacccctcccctcccccccggggggaccccccgccggggctccccccggggggagatgcgcgagctgttctcggagcacctactcaatgtcgaggggaggccccacctcttcaaagaggggcctgcacagcctccctcttaatctaaacttcccctctgaagactggatcatcagtgaagcctgcgatctactgtagcagagactatagcctttaaaatctgcccagaagtcaattccggagaacactctagtaaggaagttttcgtagccttcaggcatgcatcgtcccacaacccctataatatcaagttgtagccagtgtagtccttggtccattctggatctcagcatcttgctcaaagatctctccaagatttcggaatgatcctccattagctcaccaataaaaacattaagatcctggtcatggatgttggggttgaagggcaggagtttcctggtcccttcaataaggtatcctgagtcaacaactaaggggatttcaacatgctcaggccctatatgcgtgaaatcatgttcctccaggtccaggatctcctcattcaataagacggacaagggtatcacatcaggaatgggggaactatccctcaagtagtcacaaatattttcagtggacagcaaccaaattgatgtggtttcccttttaaaccatgtagatgtctgtgcaactctccagcaatctttaattgcggatctttgaacactaccactgtaagttacatcaagggaatgaggcttgaataatccaccttccaacaccacccctttgaaaaggaggtggagactaaatttctctttcaatgattcaccctgacttctcacctgaaaatttactgtgacagaaatttgaacgctcccaacactgaggtcggcaaaagcttcaagcagctcacttaccgactcaaagacgtgctgctcggttaatcctgatgctatgagctgtcctaagtttagaattgctggtgcacattcaaaatccagccaagcataacctgagtaaagatcatgcctgtacatgggtctttcaacacccttataaataaccttttctataaaagaagaaaaggggccgaggttgtcaagcccttccgactcaggaattgaagatctatgtggaaaccaagtaaaagaacctaaagtcctgcttgttgcaacgaatggtctgatgaaggactcaaagtagatttgtctcaggaaattttgtaccacatcataagtggtcaccaccctttcatccgtgattgggagatgagacctctcatgtgctattgctaaatctgaagacagaaccacataatgctcttctctcttccacttgacaacatgagaaattaatgacattgcctcacaattcacatctagaaccacacaaagatcaagaaacttaatccttggtgtccacctcattttcgttgatgctaggtcactcatgaagggcaagaggtgtctctcaaatagagatgggtaaagtcttctgaatgagtgaataatatggttaaaccccaccttgtcttccaagagttttgtggtagtgggcttcattgggaaatagtcacatggcttgaagggaattgaggatgttttctccttcactttgggttcacccaacacccaggaacccagctcaagtgatgtagataaggctatgcagaagtaatcccaaaggacaggtctcaagaaacctaacgcttcctcagctgcctttgtgtgctccttacccaagatccaaagatcgacgcccttaactttcaagctggaaacagaatccaatgctgacagtcttgaaactaatgattttatataggcggtggacctatctgagaatcgaacacatttgctacctagtgtcttgcaaagacctatgaaacctgatgctatcccactctgaaaggcacttttgttgacggcttcacagagcaacttttgtgcccctcttgtgaaattagaggataacttgctttgcaatgttttcaccaaggtaggcagtttttcttccccgagttcaacagcacctgtgtaaatgaccttctgtctcatgaccatctttaaagggtgatgagttgtcaaattaagccaacacaccgataaatcctcttctgagagggtgtcatcaccaacaaggctggtaagtttcttgaaggaatctgcggggtctcctgacaagtagatggctgtgaattcttcatgaagctctcctaatttcagcttattaaaaactgtccttaggaccttcctaactttctgagtgctttctggtaggattgcctcaatgttcctcataatcctatagcccctattgccatcaacccaatctttaacatcagaatccaaaaacaacaagaaaggatcaatgggatattgtgcatatctaagtagattcagtgttctctcctgaatagcgttacatagtgtgacaggcacaccattggccactgactgatcaatgatagtgtcaatagtttcagctaattgatgtggctccttacacttaacgttgtggagggcggcagccacgaatttcgttaacaatggaacttcatcaccccagacataaaaccttgatttgaactcagcaacaaatgaaccaataacactctttggactgatgaatttattcaattgatcacttaggaaatagtgaaattctagaatggcttggaattcctcaggctcatttgagagtaaatctgaaagatccttgccaaatagtgatatttgatcatcactagaggtatatgaatcaacttcatcttcaaatagacacttaatgcaataatttatgaatctctcactcactagaccgtagaagtcagaagcattgtgcaagatcccttgccccatgtcaattattgagctgagatgtgatggtatcctgttttgtttaaagtattcaaagaagaaagcttctgttgacgtttggtggagtgattttttcaaacctaacctagacttaagataagatctcatcattgcattcacaacattgaaaggtacctcaattaacttgtgaatgtgccacgacagcaaagtggacacgtaatccttactttttatgtcctctatatcatctcctgtctcccatcgcaaattttgaagaaacaccaggaaaagaaaaggggacatcataggcccccacttgctgtgatcgaggctataagagacctgacctagtgaaacattcaatttcatggctagaatggcattgtggaattccttttcattattaagacagctccctctatattgcttagttagtgcctcaaaataatcctcaattaggcgtgtgaacatttttgtcctcaggtcacctatataaagttccctgttccctccaacttgttctttgtaagataaggaaaacttgagccttccagtatcaggacccaaggatgtgtatgactgtggggattcttgactgtaaaaacataggttctttagaactgcagttgtacaatttgtcaatgacagtgctttacctaatgcctcagaattgctctctctttcactgatcctaacatcattcatcagcctttctctatcaatcttgaagtttaagcatttgtttttgtaatggctgtattttccagccaatttccctgcattcatctgaagaagtagtgacttgaagcactgaaaaaactgctggtcattgaatgttctggtggagacagcttgggtgatgcatgttattgggcaagagtccaaggaatccaaatagaaatactttgaccttaactccaaatccttaaaaaagcctttgcagatcatctcataagcatctccaggtagtagtgactcatcaaaatcttccaccatgtggtgtgctatctctgcctggatcagtctgtagtaaacacagcccacatcttcagtacctaaaaccttcctcaagtctgtcaaggacttctcctctgacccatttcccacagggaacttcctgtcatatctatacttctcaaggacgacatcaacagatttcttcacatcctctagacagtcaagtgcacaactatccaagaataactcatctaattcgtcaggagccccttgtgaattgggatttgaggaacctgacttcttccccacaacaaggtcactaatggccttttgcaccttataatcataatcctccttactcaagaggtatttagttttcctctggaatacctctgatagctggcaaacagaagcagccactagtttatcatgatcgtaattaagtatcctctctccgtcagtatacttattcacaaccacacttttgttgcttgcaaggtcaagagctgttgcacacccagcagtaactaatggatctttaaacccacttaggtcatctcctttcttgaaaagagagccattgttaaaagaagacgtcatcattgagaagaattttcttgacacaccaggaactttataagatattctgttgttactcatactggtttctttggataaaaatttgtttgctgattcaagcagcagcatcctttcatcttcatccccctcttcagatgggttgatgttaatactgtcaaactccaacttgggctccaaatacttctcaaagcacttaatctgatctgtcaacctatctggagtttccttagttataaaatgacacatgtatgataggtttaacacaaacttaaatctattagtcaacatagtgctcaccccttcacccaggatgatgttgagaatgctcctcaccacactatacaagaggtgctccggctctgttatgagatcctccctcaacttttccattaactgcttatggtggaagtctgaaacaaaggccattatgaagtatcgcagaccttgcaagaacttctgtgctcttttgctagggttcacaagtatgaggatcatggtgattttgagcaataattttaaagttttttgttgatctctgagttctatacattcagacaaccaactcatcatggtgttgatagtctcatagataacacagtgtgagaaaattgcaggaaaaaacctttttggatctgcatagaatgaacaaatctcccccttctcaggattgttaatggcataacattttgagcactctcctgttttctgataaattaagttgaaattcccgcttcccatgataaagctttgattaaaagcttccttacaacgtacacgtttgtaccttgcagcaccatactcattttgcctcactttggctactgatgatgtcttcattgaattcaccaatgccaaagagatgctagatagtcttctcaggtcttctatggaatcagcttccaactcaataagagggtaggggaattgattcccctcttctttctcatagctcattgttggctgaatccctcttttctccattaacataagcctctcgtaactgtcctcctccagtatatcttcctctttaacataatctctaaagcacaaactggcagaggataatgcctctttggcctttgtgaggagtttgtttatgatgtcaaagaatataagctcagatctctgacacagagttcctacacctcccttcttcttcattgcactcaccattctcctttcaatccactttttaaggtccttttgtgtttcttgtagtgacaccaatctgtcgttcacacttaagaatgaggagccaagccattctgatcctaacagctcgccgtgacatttaagatcctttaagtgggccaaaaggatgattgcgtcaaaagtcagcattagttttcttctggtgttcaataactttagtgattttattttattcaacagtgacttccatccaggcacagagctttggaagtgattctgcaagcagctgtctcgatcaactccctcttgcctataacggtcgtagtcaccatagagcgctgttaccataggcgaggattttacaaacctctcttttagaggatcaatctctgagggtgtgtccccctctctgtcaatataaaggttgttgaaatctctcaagagctgtcgcctatctgtcctgacaaattgtctttctatctcaccctctttaagcttattcctaaaagcatgaaattctgatacaatctcttttttcacatcaagaatggacattttgttattgatgcctctgtggcagagctccaacaccctctcataatgatcagatcgagaatctgtcaaaacattgaggctctcaatgcccgatgttctcactcctgcttttgttaaagattcacaaagccttgagtactcagattcttcaaacatttttgatgactgctgcgagaattcaagtaagctaaataggatatgcctaaacctctcattggcccattctggaaacaaatcagtgtagaaattggttctgccgtctatcaagggcaacagaatgatgtcaacagatgataagtcaggctttaaagactctagcttaatggtatcttctttatacttttgctcaaagtttattggtgaagtcctgacaaagcattccagtaatattaatgtgttcccactgaatttgaatccatctggcaccacaaatggtagaccagggcacagaacaccttgtctctgcagaaggatttcaacagaaaggtcttctgagttgtgctcacaaccattggctttgcaactgtctatctcaatacagagtgacaacaacttaaggccctcaattaatagcattctcggctctgtctgaactaggaaggctagcttctgttttgaaagcctctcatcctctaagaggtacctgctcacaagatctttgctctcggacagcaactcctccatcaccacacacttgttcctcagaagagagagtcacaatgcctaggatcctcggtgcg

SEQ ID NO: 4 - protein, 496 aa, Machupo virus
  REGION 1..496: note = MISC_FEATURE - AAT40451.1 glycoprotein precursor [Machupo mammarenavirus]
MGQLISFFQEIPVFLQEALNIALVAVSLIAVIKGIINLYKSGLFQFIFFLLLAGRSCSDGTFKIGLHTEFQSVTLTMQRLLANHSNELPSLCMLNNSFYYMRGGVNTFLIRVSDISVLMKEYDVSIYEPEDLGNCLNKSDSSWAIHWFSNALGHDWLMDPPMLCRNKTKKEGSNIQFNISKADDARVYGKKIRNGMRHLFRGFHDPCEEGKVCYLTINQCGDPSSFDYCGVNHLSKCQFDHVNTLHFLVRSKTHLNFERSLKAFFSWSLTDSSGKDMPGGYCLEEWMLIAAKMKCFGNTAVAKCNQNHDSEFCDMLRLFDYNKNAIKTLNDESKKEINLLSQTVNALISDNLLMKNKIKELMSIPYCNYTKFWYVNHTLTGQHTLPRCWLIRNGSYLNTSEFRNDWILESDHLISEMLSKEYAERQGKTPITLVDICFWSTIFFTASLFLHLVGIPTHRHLKGEACPLPHKLDSFGGCRCGKYPRLKKPTIWHKRH

SEQ ID NO: 5 - protein, 488 aa, Sabia virus
  REGION 1..488: note = MISC_FEATURE - YP_089665.1 glycoprotein precursor [Brazilian mammarenavirus] (Sabia virus)
MGQLFSFFEEVPNIIHEAINIALIAVSLIAALKGMINLWKSGLFQLIFFLTLAGRSCSFRIGRSTELQNITFDMLKVFEDHPTSCMVNHSTYYVHENKNATWCLEVSVTDVTLLMAEHDRQVLNNLSNCVHPAVEHRSRMVGLLEWIFRALKYDFNHDPTPLCQKQTSTVNETRVQINITEGFGSHGFEDTILQRLGVLFGSRIAFSNIQDLGKKRFLLIRNSTWKNQCEMNHVNSMHLMLANAGRSSGSRRPLGIFSWTITDAVGNDMPGGYCLERWMLVTSDLKCFGNTALAKCNLDHDSEFCDMLKLFEFNKKAIETLNDNTKNKVNLLTHSINALISDNLLMKNRLKELLNTPYCNYTKFWYVNHTASGEHSLPRCWLVRNNSYLNESEFRNDWIIESDHLLSEMLNKEYIDRQGKTPLTLVDICFWSTLFFTTTLFLHLVGFPTHRHIRGEPCPLPHRLNSRGGCRCGKYPELKKPITWHKNH

SEQ ID NO: 6 - protein, 484 aa, Chapare mammarenavirus
  REGION 1..484: note = MISC_FEATURE - YP_001816782.1 glycoprotein precursor [Chapare mammarenavirus]
MGQLVSFFQEIPNIIQEAINIALIAVSLIAILKGLVNLWKSGLFQLLVFLILAGRSCSFKIGRSTELQNITINMLKVFEDHPISCTVNKTLYYIRESENATWCVEIAALDMSVLLSPHDPRVMGNLSNCVHPDIKHRSELLGLLEWILRALKYDFLNYPPLLCEKVTSSVNETRIQINVSDSAGSHDFKETMLQRLAILFGTKLMFDKTPKQFIVIRNQTWVNQCKSNHVNTLHLMMANAGHAVKLRRLQGVFTWTITDAAGNDMPGGYCLERWMLVTSDLKCFGNTALAKCNLNHDSEFCDMLKLFEFNKKAIESLNDNTKNKVNLLTHSINALISDNLLMKNRLKELLDTPYCNYTKFWYVNHTITGEHSLPRCWMVKNNSYLNESEFRNDWILESDHLLSEMLNKEYFDRQGKTPITLVDICFWSTLFFTTTLFLHLVGFPTHRHIQGEPCPLPHKLNSNGGCRCGRYPELKKPTTWHRKH

SEQ ID NO: 7 - protein, 485 aa, Junin virus
  REGION 1..485: note = MISC_FEATURE - ABI51601.1 glycoprotein precursor [Argentinian mammarenavirus] (Junin virus (JUNV))
MGQFISFMQEIPTFLQEALNIALVAVSLIAIIKGIVNLYKSGLFQFFVFLALAGRSCTEEAFKIGLHTEFQTVSFSMVGLFSNNPHDLPLLCTLNKSHLYIKGGNASFMISFDDIAVLLPQYDVIIQHPADMSWCSKSDDQIWLSQWFMNAVGHDWHLDPPFLCRNRTKTEGFIFQVNTSKTGVNENYAKKFKTGMHHLYREYPDSCLNGKLCLMKAQPTSWPLQCPLDHVNTLHFLTRGKNIQLPRRSLKAFFSWSLTDSSGKDTPGGYCLEEWMLVAAKMKCFGNTAVAKCNLNHDSEFCDMLRLFDYNKNAIKTLNDETKKQVNLMGQTINALISDNLLMKNKIRELMSVPYCNYTKFWYVNHTLSGQHSLPRCWLIKNNSYLNISDFRNDWILESDFLISEMLSKEYSDRQGKTPLTLVDICFWSTVFFTASLFLHLVGIPTHRHIRGEACPLPHRLNSLGGCRCGKYPNLKKPTVWRRRH

SEQ ID NO: 8 - protein, 479 aa, Guanarito virus
  REGION 1..479: note = MISC_FEATURE - AAN05423.1 glycoprotein precursor [Guanarito mammarenavirus]
MGQLISFFQDIPIFFEEALNVALAVVTLLAIIKGIVNVWKSGILQLFVFLVLAGRSCSFKVGHHTNFESFTVKLGGVFHELPSLCRVNNSYSLIRLSHNSNQALSVEYVDVHPVLCSSSPTILDNYTQCIKGSPEFDWILGWTIKGLGHDFLRDPRICCEPKKTTNAEFTFQLNLTDSPETHHYRSKIEVGIRHLFGNYITNDSYSKMSVVMRNTTWEGQCSNSHVNTLRFLVKNAGYLVGRKPLAFFSWSLSDPKGNDMPGGYCLERWMLVAGDLKCFGNTAVAKCNLNHDSEFCDMLRLFDFNKNAIEKLNNQTKTAVNMLTHSINSLISDNLLMRNKLKEILKVPYCNYTRFWYINHTKSGEHSLPRCWLVSNGSYLNESDFRNEWILESDHLIAEMLSKEYQDRQGKTPLTLVDLCFWSAIFFTTSLFLHLVGFPTHRHIQGDPCPLPHRLDRNGACRCGRFQKLGKQVTWKRKH

SEQ ID NO: 9 - DNA, 3439 bp, Machupo virus
  misc_feature 1..3439: note = Machupo virus strain Carvallo segment S, complete sequence Genbank access number AY619643
  source 1..3439: mol_type = unassigned DNA
cgcacagtggatcctaggcgattcttgatcgcgcttattagctaaccaatttaatttggtgttgaagtgttgacacgctctctaacacatggggcagcttatcagcttctttcaggagattcctgtttttctacaggaagctctgaacatcgctttagtggctgttagtctcatagctgtcatcaaaggcatcattaacctttacaaaagtggtctcttccagttcatcttctttctcctcctagcagggaggtcctgctcggatggcacattcaaaataggcctacacactgagttccagtcagtcacccttaccatgcagagacttttagctaaccattcaaatgagctcccatctctctgcatgcttaacaatagtttttattatatgaggggaggtgtgaacaccttcctgattcgtgtttctgatatttcagtcctcatgaaggagtatgatgtatcaatctatgaaccagaagaccttggaaattgtcttaacaagtctgactcaagctgggctattcattggttctcaaatgctttgggacatgactggcttatggatcctccaatgctatgtagaaacaagacaaagaaggagggatctaacattcaattcaacatcagcaaagctgatgatgccagagtgtatggaaagaagataagaaatggtatgaggcatctcttcaggggcttccatgacccgtgtgaggaagggaaagtgtgctacctgaccatcaatcagtgtggtgaccccagttcctttgactactgtggcgtgaatcatctttccaaatgtcagtttgaccatgtgaacacccttcatttccttgtgagaagtaagacacatctcaactttgagaggtctttgaaagcatttttctcatggtctctgacagactcctcaggaaaggacatgccaggaggttattgtctagaggaatggatgttgatagcagccaaaatgaaatgtttcggaaacactgctgttgctaaatgtaatcaaaatcatgactcagagttctgtgatatgctgaggctattcgactataacaagaatgcaataaagaccctcaatgatgaatcaaagaaagaaatcaatcttctaagccagacagtgaatgccttaatctcagataatttgttaatgaagaataaaattaaagagctaatgagcatcccttattgtaattacacaaagttttggtatgtcaatcataccctgacagggcagcacactcttccaagatgttggttgataaggaatggaagttatcttaacacttctgaattcaggaatgactggattttagagagtgatcacctcatctcagagatgttaagtaaggaatatgctgaaaggcaaggcaaaaccccaatcacattagttgatatttgtttctggagcacaattttcttcacagcatcattgttccttcatctagtcggaatacccacccatcgacacctcaaaggcgaagcctgtcctttgcctcataagctggacagcttcggaggttgtagatgtggcaaatatcccagattgaagaaacccaccatctggcacaaaagacattaaacacagccaagacccctgccgacccgggcccagcccgggtcggcggggccccccagtccgcggctctgccgcggactggggaggcactgcttacagtgcaaaggctgccttgggtagaaatagaaggctgagaggtaaaagtgccactggttcttcatctaccattttgccatccatcactgattgatacatgatacaatccaacagagcacagtgaggttcaccagaactcaatttgaatggagagtctttttctctctttttcttgttgacaacaactccattatgatgtttgcaaagatgtccaaacttctcccaaatttgctgatcatagagtcttgcttgatcagatgtgagtttcacatctaccagctttaaatccttccgtccatgaatgtctaaaagtttccttatgtcgtcagaaccttgagttgtaatcaccatatcttgtggtagtaaacctattacataacttaatactcctggcattgcatcctcgatgtcttgcatcaagatgccatgtgaatgtctgctgccatttttgaagcccttctcatcatgtggctttctaaaacaatgaatataatgcttgttggctggctgatatagtgccaattccacagggtctgttggaggcccttcaatgtcaagccacagtgtattagtggggtctaacttctttacagcctctttaatcactgcttcttgcatttcagtcaggtttgctagtcttagattctgaccgtttttctcgggttgtctcggtccaacttgaggtttctttgttagatcaacacttgtgttgtcccaagatctcccaagaatttgtgatctggaaccaatgtaaggccacccgtcacctgagagacacaatttatacagaaggttctcataaggatttcgattcccaggtttctcgtctataaacatcccctcttttcgcttgacttgcagagttgatttaatcaagctggagaaattatcaggagtgaccctaattgtttccagcatgttcccaccgtcaagtattgaggcgccagctttcacagcagctgacagactgaagttatagccagagatgttgatggagctctcgtccttagttacgatctgcaagcattcgtgctctagagttagcttgtcaaggtcatttaaattcggatatttaacagtgtataacaggcccagagatgtcaacgcttggaccacactattcatcgtttctccaccctggacagtcatacaagcgattgtgagagctggcatggaaccaaactgattattcagttttgatggatctgagacatcccagattctaaccacaccatttccagcacctctctgctgttggaatcccagggtcttcaagatttctgacctctttgtcagttgcagctgtgacaaattacccatgtacagcccttggggacctgactcagtcctctttatcttgttcttcagtttttccaagtctgatgcaagctccatcaattcatctctccccagatctccaatcttgaatatggtgtttttctggatgcttttcatagacatgagcctatccacttctttgtttaaatccctcagcttgttcagatcctcttcaccccttttgttctttctgagagccctctgcacttgtgaaacctggttgaagtcgatgctgtcagctatgagcttggcatctttcaacacatctgttttcacagtgtgggtgaactgactcaggcctcttctcagtgactgagtccaccgaaagctgggaatttccttggagtgagccatagtggctaataaccaaaggatttgaaataaagctcagtagaagagttagacactttgcctaggatccactgtgcg

SEQ ID NO: 10 - DNA, 3366 bp, Sabia virus
  misc_feature 1..3366: note = NC_006317.1 Sabia virus, complete genome
  source 1..3366: mol_type = unassigned DNA
gcgcaccggggatcctaggcgttttttagtcacgcttaaatctttgattgcgtcaatcatgggtcaattgttcagcttttttgaagaagttccgaatatcatccatgaggctatcaacatagctctgatagcagtgagcttaattgctgccttgaaagggatgattaacttgtggaagagtggccttttccaattgatattctttttgactctagcaggaagatcgtgttcttttagaattggaaggagcacagaattgcaaaacataacgtttgatatgttgaaggtattcgaggaccaccccacatcttgcatggtgaatcattccacctactatgtccatgaaaacaaaaatgccacttggtgtcttgaggtgtctgtgactgatgttaccctgctcatggctgaacatgatcgtcaagtcctcaacaatctgtcaaactgtgtgcaccctgcagttgagcacagaagcaggatggttggcttacttgagtggatttttagagccctaaagtatgacttcaatcatgatccaacaccgttgtgtcaaaagcaaacttcaacagtgaatgaaacacgtgtgcagataaacatcactgaggggtttgggtctcacgggtttgaagataccatccttcaaagactaggggttctattcggttcaagaattgcattttcaaatatccaggatttaggtaaaaaaaggtttttattgattagaaattcaacttggaaaaatcaatgcgaaatgaatcatgtaaactccatgcatttaatgttggcgaatgctggtcgctcttctggttctagaagaccactcggcattttctcctggacaataactgatgcagtgggcaatgacatgcctggtggttattgtcttgaaagatggatgctagtgacgtcagatcttaagtgctttggaaacacagcactagcaaaatgtaaccttgaccacgattcggaattctgtgacatgttgaaattgtttgagttcaacaaaaaagcgatagagacattgaatgacaatacaaaaaacaaggtaaacttgctgacccactcaattaatgcattaatatctgacaacttactgatgaagaatcgacttaaagaattgttgaacactccttattgtaattacaccaaattttggtatgtcaatcacacagcatcaggggaacactcattgccacggtgctggcttgttagaaataatagctacttgaatgaaagtgaatttagaaatgattggattattgagagtgatcatttattgtctgaaatgctcaataaagaatacatagatagacagggaaagacaccgttgactttggtggatatctgtttctggagcactttgtttttcacaacaacactgtttcttcacctggtaggctttccaactcatagacacatacgtggtgaaccctgcccactaccccataggctcaacagtagaggaggatgtagatgtgggaaataccctgaactaaaaaagccaatcacctggcacaagaaccactagaaggacaatcattgtctcccaccgacccgggcaatgcccgggtcggtgtggccccccagtccgcggcaaatgccgcggactggggagcaccaatctagatggtgaatgctgcctccagtgtgaagagcaatgcatcaggcaataaaccttccagtttggtttgaggtggttggcctgctatggctgactgaaacattagacaatcaagtagggcacagtgtggctctgaggactgtgggatgtctttgtctttctttttctttggcacaataactccattgtgtttcttacagaggtgaccaaaatctgaccagattggctcctcaaactttctcgcctgttcagatgccaatttaatgtcgacaatctttaagtctttgcgaccgtgtgtatctactaagcgccttatatcatctgaaccttgggtggtgaggaccatgttttgaggaaggagccctataacatagctgagcaagcctggctgtgcagattctaggtcctttagcaacatcccatgtgaatgcctgctaccatttttgaaccccttctcatcatgtggtttcctataacagaggatataattacctgtgtctggttggtacaatgctaattcaaccggatcattactggtaccctcaatgtcaatccatgttgtcagagttgggtcaatcttacccattgcctctctcacaactgactcttgcaactcagtcaagtgggagagtctaatgttctgacctgccttttctggtgttctcggcccttgttggggctttgtgcttaaatcaacagtggtgttttcccatgacctacccttgatctgggatctggagccaatgtaaggccaaccttctcctgaaaggcagattttgtacagaaggttttcataagggtttctatttccaggtttctcatctacaaacatgccttcctttttctttatggatagggttgtctttatgatctgagaaaagttgtcaggagtgatccttatggtttccagcatgttaccaccatccagaagcgttgcaccagctttaacagctgcagaaagactatagttatatcctgagatgttcaagccgctctcatctttagtcactatttgaagacagtcatgttctgctgtaagtttttctaggtcaatcaggttggggaacttaactgtataaagaagtcccaaagatgttagtgcctgaacaacatcccccatggtattgtcactttgtttagtcatacaagcgattgtcagtgcagggacagatccaaattgattattcagcctgttcggatcagctacgtcccaaatccttacaacccctggagacctcacttgctgctgttgaaaaccaagtttccttaaaagatcagaccttttggtgagctgtgattgggaaaggtttcccaggtacacacctcctgaggctgatctttctgtttgtccaacttttcttttcagtttctccaggtctgatgcaagatccatcagttcagatttgttgagatcaccaagttttaagattgtgtctctttgggaactcttcatgctcatcaggttgtccacttctttatttaggtctctcagtttatcaagatctccatcattccttttagactttctaaggatcctttgaacaagagagacttgattgaaatcaagaccatcaagtatcattttggcatccctcagaacatcggtcttcaccggtgttgtgaactcactgagccctcttctcagggattgagtccatctgaagctggggatttcctttgagttgctcattgtggaaaagacttgctgaccagtaaaggtagacaatttgcctaggatccactgtgcg

SEQ ID NO: 11 - DNA, 3357 bp, Chapare mammarenavirus
  misc_feature 1..3357: note = NC_010562.1 Chapare virus segment S, complete sequence
  source 1..3357: mol_type = unassigned DNA
tcgcacagtggatcctaggcgctttttggtcacgcataaatcgtgagcaagaataaacatgggtcaacttgtgagtttctttcaggaaattcctaacatcatacaggaagcaatcaacattgctctaatagctgtcagcctcattgctatcctaaaagggttggtcaatctgtggaaaagtggtttgtttcaactcctggtcttccttattctagctggaaggtcctgctcattcaaaattggaagatccacagaactccaaaacatcacaataaacatgttaaaggtctttgaggaccatcctatttcttgcacagtgaacaaaactctttattacatccgtgaaagtgagaatgcaacatggtgtgtagagattgctgcactggacatgtctgtcttgctctcgccacatgatccacgtgtaatgggcaacctatcaaactgtgttcacccagacattaaacatagatcagaacttctaggtctattggagtggattttgagagctctgaaatatgattttctaaattatccacccctcttgtgtgaaaaagtgacatcatctgtcaatgaaacacgcattcagataaatgtgagcgatagtgcaggttctcatgatttcaaagaaaccatgctccaaaggctggccatactatttggcacaaaattgatgtttgataaaacacctaagcagttcatagttatcagaaatcagacttgggtgaatcagtgcaagtccaatcatgtcaacacgttgcatttgatgatggctaatgctgggcatgctgttaaattaagaagattgcaaggggtgttcacttggacgatcacagatgctgctggcaacgacatgcctggaggttattgtttggaaaggtggatgttagtcacatctgacctgaaatgttttggcaacactgctcttgcaaagtgtaacttaaatcatgactctgagttctgtgacatgttgaaactgtttgaatttaacaaaaaagcaattgagtcattgaatgacaacacaaagaacaaagtcaatctattaacacattccattaatgctttaatttctgacaatcttttgatgaagaatagattaaaagagttattggacactccctactgcaactacacaaaattctggtatgtcaaccacaccatcacaggagagcattcacttccacgctgttggatggttaaaaacaacagctacctcaatgaaagtgaatttagaaatgattggattcttgagagtgaccacttgttgtccgaaatgttgaacaaagagtattttgacagacaagggaaaaccccaataacacttgttgacatctgcttttggagcacactgttcttcacaacaacattgtttctccatctagtaggcttcccaactcacagacacattcagggagaaccttgcccactgcctcacaagctcaacagcaatggtggatgcaggtgtggcagatatccagaactcaagaaaccaacaacctggcacaggaaacactgaacttgagtggctcccaacccaccgtcccgggcatagcccgggacggtgcggcctcccagtccgcggcaaatgccgcggactgggagggcatgggcctatatggcgaatgccggcttcattgtaaacagcagcacttctggtagaagactctgcagtttgatctgtggtggctgcccatctatcaccgactggaacatcagacagtcaagcagggcgcaatgtggttctgttgattgtgaggggtctttgtcttttttcttttttggcaccacaaccccattgtgcttcttacagaggtggccgaaatcagaccagacctgctcctcatagtttctggcttgatcagaacttagtttcacatccacgaccttgagatctttgcgaccatgtgtgtctaaaagtcttttgatgtcatcagagccttgggcggttaggaccatgtcttgtggtaacaaccctatgatgtagctaaggagtccaggttgggccgattccaagtcctttagtagcattccatgtgaatgtctacttccattcttgaatcctttctcatcatgaggttttctgtaacagagtatgtaattacctgattccggttgatataatgctagctcaacagggtcattgctggttccttcaatgtcaatccatgttgtgtgggaaggattaattttgctcatcgcctctctaacaactgactcttggatctctgtcagatgagagagtctcacatttaaccccactttctcaggtgctttcggtccttgtgttggcttcaagctaagatcaacagttgtattttcccaagacctacctttcacttgagacctggagcctatataaggccaaccttcacctgataggcacaatttatataataggttttcataggggttcctgtttcctggtctctcatcgataaacatattttctcttttctttacacccaaaactgtcttgataatggttgagaaattgtctggagtcacctttattgtttctaacatgtttcccccatccagaaaagtggccccggccttaacagctgctgaaaggctgaaattgtatcctgatatattgattgcactttcatccttagtgatgatctgaaggcaatcatgttcggcagccaacttttccagatcagctaggtttgggaacttcacagtgtaaaggagacctaatgatgttaatgcttggacaacatttcccattgtatcgcccccctgaacggtcatgcaggctattgtcagtgcaggaactgagccgaactggttgttcagtctggtgggatcagccacgtcccaaattctaacaacacctgtggatctcacctgtggttgttgaaatcctagtctcctcaaaagatcagttcttttggataactgtgattggctaagattcccaaggtagaccccacctacaggagatctctctgtctgtccaactttctttttcaacttttctagatcagatgcaagatccatgagctcagatttgttgagatcaccaagcttcaggatggtgttcttctgagcacttttcatggccatcaggttgtccacttctctattgaggtctcttagcttgtccaaatctccatcggttctttttgtttttctaagaactctctgaacaagtgcaacttggctaaaatcgagaccatctgcaatcattttagcatcttttagcacgtctgacttcacaggaattgtgaaactgctcaattctcgtcttaaagactgagtccagcggaaacttggaatctcttttgagttgctcatgatggaaaggctttctgataagaaattgcttaccaaaaatgcctaggatccactgtgcga

SEQ ID NO: 12 - DNA, 3409 bp, Junin virus
  misc_feature 1..3409: note = DQ854733.1 Junin virus strain P2045 segment S, complete sequence
  source 1..3409: mol_type = unassigned DNA
cgcacggaggatcctaggcgattttggttacgctataattgtgactgtttactgtttggacaacatcaaaacatctattgcacaatggggcaattcattagcttcatgcaagaaataccaacctttttgcaggaggctctgaacattgctcttgttgcagtcagtctcattgccatcattaaggggatagtgaacttgtacaaaagtggtttattccaattctttgtattcctagcgcttgcaggaagatcctgcacagaagaagctttcaaaatcggactgcacactgagttccagactgtgtccttctcaatggtgggtctcttttccaacaatccacatgacctacctttgttgtgtaccttaaacaagagccatctttacattaaggggggcaatgcttcatttatgatcagctttgatgatattgcagtgttgttaccacagtatgatgttataatacaacatccggcagatatgagctggtgttctaaaagtgatgatcaaatttggttgtctcaatggttcatgaatgctgtgggacatgattggcatctagacccaccatttctgtgtaggaaccgtacaaagacagaaggcttcatctttcaagtcaacacctccaagactggtgttaatgaaaactatgctaagaagtttaagactggcatgcatcatttatatagagaatatcctgactcttgcttgaatggcaaactgtgtttaatgaaggcacaacccaccagttggcctctccaatgtccactcgaccatgttaacacattacatttccttacaagaggcaaaaacattcaacttccaaggaggtccttgaaagcattcttctcctggtctttgacagactcatccggcaaggatacccctggaggctattgtctggaagaatggatgctcgtagcagccaaaatgaagtgttttggcaatactgctgtagcaaaatgcaatttgaatcatgactctgaattctgtgacatgttgaggctttttgattacaacaaaaatgccatcaaaaccctaaatgatgaaacaaagaaacaagtaaatctgatggggcagacaatcaatgccttgatatctgacaatttattgatgaaaaacaaaattagggaactgatgagtgtcccctactgcaattacacaaaattttggtatgtcaaccacacactttcaggacaacactcattaccaaggtgctggttaataaaaaacaacagctatttgaacatctctgacttccgtaatgactggatattggagagtgacttcttaatttctgaaatgctaagcaaagagtattcggacagacagggtaaaactcctttgactttagttgacatctgtttttggagcacagtatttttcacagcgtcactcttccttcatttggtgggcatacctacccacagacacatcaggggcgaagcatgccctttgccacacaggttgaacagcttgggtggttgcagatgtggtaagtaccccaatctaaagaaaccaacagtttggcgtagaagacactaagacctcctgagggtccccaccagcccgggcactgcccgggctggtgtggccccccagtccgcggcctggccgcggactggggaggcactgcttacagtgcataggctgccttcgggaggaacagcaagctcgatggcaatagaggtgtaagttcctcctcatagagcttcccatccaacactgactgaaacattatgcagtctagcagagcacagtgtggttcactggaggccaacttgaagggagtgtccttatctctctttttcttattgacaaccactccattgtgatatttgcataggtgaccatatttctcccagacctgttgatcaaactgcctggcttgttcagatgtaagcctaacatcaaccagtttaagatctcttcttccatggaggtcaaacagcttcctgatgtcatcggatccctgagtagtcacaaccatgtctggaggcagtaagccgatcacataactaagaactcctggcattgcatcttctatgtccttcattaagatgccgtgagagtgtctgctaccatttttaaaccctttctcatcatgtggttttctgaagcagtgaatgtactgcttacctgcaggttggaacaatgccatctcaacagggtcagtggctggtccttcaatgtcgagccaaagggtattggtggggtcgagtttccccactgcctctctgatgacagcttcttgtatctctgtcaagttagccaatctcaaattctgaccgtttttttccggttgtctaggaccagcaactggtttccttgtcagatcaatacttgtgttgtcccatgacctgcctgtgatttgggatctagaaccaatataaggccaaccatcgccagaaagacaaagtttgtacaaaaggttttcataaggatttctattgcctggtttctcatcaataaacatgccttctcttcgtttaacctgaatggttgattttatgagggaagagaagttttctggggtgactctgattgtttccaacatgtttccaccatcaagaattgatgctccagcctttactgcagctgagagactgaagttgtaaccagaaatgttgatggagctttcatccttagtcacaatctgaaggcagtcatgttcctgagtcagtctgtcaaggtcacttaagtttggatacttcacagtgtatagtagcccaagagaggttaaagcttgtatgacactgttcattgtctcacctccttgaacagtcatgcatgcaattgtcaatgcaggaacagagccaaactgattgtttagttttgaagggtctttaacatcccatatcctcaccacaccacttcctccattcccttgctgttgaaatcccagtgttctcaatatctctgatcttttagcaagttgtgactgggacaaattacccatgtaaaccccttgagagcctgtctctgctcttcttatcttgttttttaatttctcaaggtcagacgccaactccatcagttcatccctccccagatctcccaccttgaaaactgtgtttcgctgaacactcctcatggacataagtctgtcaacctctttgttcaggtccctcaacttattgaggtcttcttccccccttttggtctttctgagtgcccgctgcacctgtgccacttggttgaagtcaatgctgtcagcaattagcttggcgtccttcaaaacatctgacttgacagtctgagtgaattgactcaaacctctccttaaggactgagtccatctaaagcttggaacctccttggagtgtgccatgccagaagttctggtgattttgatctggggtagagttgctcagtgaaagtgttagacactatgcctaggatcctckgtgcg

SEQ ID NO: 13 - DNA, 3343 bp, Guanarito virus
  misc_feature 1..3343: note = AY129247.1 Guanarito virus strain INH-95551 segment S, complete sequence
  source 1..3343: mol_type = unassigned DNA
cgcacagtggatcctaggcgtttttactcacgcaataatttgtccacactattgttgggtgtgacctagcataatgggacagttaatcagtttctttcaggacataccgatcttctttgaagaagcactcaatgtagccttggctgtggttacactgcttgctattataaagggcattgtgaatgtttggaaatcaggaattcttcagctgtttgtgttcttggttctggcgggaaggtcatgctctttcaaagttggtcatcatacaaattttgagtcatttacagttaagctaggaggtgtcttccatgaattaccttcactatgtagggtcaacaactcttacagtctaattaggctttctcataacagtaatcaggcattatcagttgagtatgtggatgtgcaccctgtcctctgttcgtccagtccaaccatactcgacaactacactcaatgtataaaaggctctccagagtttgattggattcttgggtggacaattaagggattgggacatgactttttgagggatccaagaatctgctgtgagcctaaaaagacaactaatgctgaatttacattccaattgaatttaacagatagtcctgagacccatcactataggagcaagattgaggtaggcatccgacatttattcgggaactacataactaatgatagctattcaaaaatgtctgtggttatgaggaacactacctgggaaggtcaatgctcaaatagtcatgtgaatacactgagatttcttgttaaaaatgcaggttaccttgttggaagaaaaccactggcattttttagttggtcactttctgacccaaagggtaatgacatgccaggtggttactgtcttgaaaggtggatgttggttgctggggatttgaagtgctttggcaacacagctgtcgccaagtgtaatttgaatcatgattctgagttctgtgacatgttgaggctgtttgatttcaataagaatgccattgaaaaactgaacaaccaaactaaaactgctgtcaacatgttgactcactcaataaatagtctaatatctgataatttattgatgagaaacaaactgaaggagattttgaaggtcccatattgcaactacacaagattctggtacataaaccacacgaaatctggtgagcactcactgcctcggtgttggctggtcagtaatggttcctatttgaatgagagtgattttagaaatgagtggatcttggagagtgatcacctaatagcagagatgttaagcaaagaataccaagataggcaggggaagactcccctaacactggttgatctatgtttctggagtgcaatctttttcaccacaagtctctttttacacctggttgggttcccaacacatagacatatacagggtgacccgtgccctttgcctcacaggctcgatagaaatggtgcttgcaggtgtggcaggtttcaaaaacttggaaaacaagtaacctggaagagaaagcattgatttcatcagccctccagctctgtcggcccggaaacccgggccgacagagcgccccccagtccgcggcaatgcccgcggactgggagggcatctgcctacatcacaaatgcgctcttggcttggaacactaatgtgtttggtagcagttgaatgggaatggtgtcgggaagatgaccatcaagaacagactgaaacatgatgcaatcgagaagagcacagtgtggctccttcgtggagttagcctccttgtttttcttcttaggaacaatgacaccattgtgtgctctgcagaggtggccaaatttttcccaaacttcctgttcaaaaattctggactgttccccagtcagtctgacatcaacaagttttaaatctttccttccatgtatgtcgaaaagctttttgatgtcatctgccccttgaacagtgataactgaaccctgtggcaataagccaatgacgtaactcaatagaccaggttgagcatcttcaaggtcctttaatagtatgccgtgtgagtgcctacttccatttttaaaacctttctcatcatgaggttttctgaaacagtgtacatagtttcctgaagaaggttggaaaactgccaactccacaggatcagttggcgggccttcaatgtccatccagattgtgtctgatgaatctaatttcctcattgcttcctttacgatcgcttcttgcatttcagaaagatttgatagtctgatattttgtccattcttttcaggagctcggggacctgttacaggtcttgcatttaaatcgacacttgtgttgtcccaagacctcccgagtatttgtgaccttgatccaatatatggccaaccctccccagacaaacacagcttgtagagaaggttctcataagggtttctattgcccggtctctcatctatgaacatgccttctcttcttttgacgttcaatgcggccttgacaatagaagagaagttgttgggtgtgacttttattgtctccagcatgttgccaccatctataagtgatgcaccagcttttacagcagctgaaagactgaagttataaccagatatgttgagtgcactctcatcctttgttataatctgtaggcagtcgtgttctaaagttagtttttccaggtcatcaagattgggatatttgacagtgtagagaagaccaagtgatgttagtgcctgcacaacattgttcattgtttctcctccctgaactgtcatgcaggcaatggtaagagctggcattgaaccaaattggttattcagtttggagggatcagatacatcccacaacctgacaaccccacctgcagcccccctttgttgttggaatcccagggttctaagaatccctgctcttttttccaattgtgactgcgtcaagtttcccatgtacatccttggactattgctttccctatctccaatcttcttctttagtttttctagatctgaggccaagtccattaattcatctttgcccaaatcaccaactttcagaactgtgttgttctgaacacttttcatgctcatcagtctatcaacttctttatttaagtccctaagtttatcaagatcagtgtctcctcgtttggacttacgtaggagtctttgaacttgagaaacttgtgtgaaatcaagggagtctgcaatgagctttgcatcattaagaacacttgatttggttggttctgtgaacatccctagttctctccttagagattgagtccagcggaagctggggatttctttggagtgagccatagttcgcgttgtgtagaagactaagacaaaaatgcctaggatcaccttgtgc

SEQ ID NO: 14 - DNA, 7272 bp, synthetic construct
  misc_feature 1..7272: note = MOPEIA L segment 7272pb in antigenomic orientation Accession number JN561685
  misc_feature 1..57: note = 5'NC region
  misc_feature 58..6771: note = RNA dependant RNA polymerase "L-polymerase" ORF
  misc_feature 6772..6881: note = Intergenic Region (IGR)
  misc_feature 6882..7193: note = Z matrix protein ORF
  misc_feature 7194..7272: note = 3' NC region
  source 1..7272: mol_type = other DNA
gcgcaccgaggatcctaggcattgtgactctctcttctgaggaacaagtgtgtggtgatggaggagttgctgtccgagagcaaagatcttgtgagcaggtacctcttagaggatgagaggctttcaaaacagaagctagccttcctagttcagacagagccgagaatgctattaattgagggccttaagttgttgtcactctgtattgagatagacagttgcaaagccaatggttgtgagcacaactcagaagacctttctgttgaaatccttctgcagagacaaggtgttctgtgccctggtctaccatttgtggtgccagatggattcaaattcagtgggaacacattaatattactggaatgctttgtcaggacttcaccaataaactttgagcaaaagtataaagaagataccattaagctagagtctttaaagcctgacttatcatctgttgacatcattctgttgcccttgatagacggcagaaccaatttctacactgatttgtttccagaatgggccaatgagaggtttaggcatatcctatttagcttacttgaattctcgcagcagtcatcaaaaatgtttgaagaatctgagtactcaaggctttgtgaatctttaacaaaagcaggagtgagaacatcgggcattgagagcctcaatgttttgacagattctcgatctgatcattatgagagggtgttggagctctgccacagaggcatcaataacaaaatgtccattcttgatgtgaaaaaagagattgtatcagaatttcatgcttttaggaataagcttaaagagggtgagatagaaagacaatttgtcaggacagataggcgacagctcttgagagatttcaacaacctttatattgacagagagggggacacaccctcagagattgatcctctaaaagagaggtttgtaaaatcctcgcctatggtaacagcgctctatggtgactacgaccgttataggcaagagggagttgatcgagacagctgcttgcagaatcacttccaaagctctgtgcctggatggaagtcactgttgaataaaataaaatcactaaagttattgaacaccagaagaaaactaatgctgacttttgacgcaatcatccttttggcccacttaaaggatcttaaatgtcacggcgagctgttaggatcagaatggcttggctcctcattcttaagtgtgaacgacagattggtgtcactacaagaaacacaaaaggaccttaaaaagtggattgaaaggagaatggtgagtgcaatgaagaagaagggaggtgtaggaactctgtgtcagagatctgagcttatattctttgacatcataaacaaactcctcacaaaggccaaagaggcattatcctctgccagtttgtgctttagagattatgttaaagaggaagatatactggaggaggacagttacgagaggcttatgttaatggagaaaagagggattcagccaacaatgagctatgagaaagaagaggggaatcaattcccctaccctcttattgagttggaagctgattccatagaagacctgagaagactatctagcatctctttggcattggtgaattcaatgaagacatcatcagtagccaaagtgaggcaaaatgagtatggtgctgcaaggtacaaacgtgtacgttgtaaggaagcttttaatcaaagctttatcatgggaagcgggaatttcaacttaatttatcagaaaacaggagagtgctcaaaatgttatgccattaacaatcctgagaagggggagatttgttcattctatgcagatccaaaaaggttttttcctgcaattttctcacactgtgttatctatgagactatcaacaccatgatgagttggttgtctgaatgtatagaactcagagatcaacaaaaaactttaaaattattgctcaaaatcaccatgatcctcatacttgtgaaccctagcaaaagagcacagaagttcttgcaaggtctgcgatacttcataatggcctttgtttcagacttccaccataagcagttaatggaaaagttgagggaggatctcataacagagccggagcacctcttgtatagtgtggtgaggagcattctcaacatcatcctgggtgaaggggtgagcactatgttgactaatagatttaagtttgtgttaaacctatcatacatgtgtcattttataactaaggaaactccagataggttgacagatcagattaagtgctttgagaagtatttggagcccaagttggagtttgacagtattaacatcaacccatctgaagagggggatgaagatgaaaggatgctgctgcttgaatcagcaaacaaatttttatccaaagaaaccagtatgagtaacaacagaatatcttataaagttcctggtgtgtcaagaaaattcttctcaatgatgacgtcttcttttaacaatggctctcttttcaagaaaggagatgacctaagtgggtttaaagatccattagttactgctgggtgtgcaacagctcttgaccttgcaagcaacaaaagtgtggttgtgaataagtatactgacggagagaggatactttattacgatcatgataaactagtggctgcttctgtttgccagctatcagaggtattccagaggaaaactaaatacctcttgagtaaggaggattatgattataaggtgcaaaaggccattagtgaccttgttgtggggaagaagtcaggttcctcaaatcccaattcacaaggggctcctgacgaattagatgagttattcttggatagttgtgcacttgactgtctagaggatgtgaagaaatctgttgatgtcgtccttgagaagtatagatatgacaggaagttccctgtgggaaatgggtcagaggagaagtccttgacagacttgaggaaggttttaggtactgaagatgtgggctgtgtttactacagactgatccaggcagagatagcacaccacatggtggaagattttgatgagtcactactacctggagatgcttatgagatgatctgcaaaggcttttttaaggatttggagttaagatcaaagtatttctatttggattccttggactcttgcccaataacatgcatcacccaagctgtctccaccagaacattcaatgaccagcagttttttcagtgcttcaagtcactacttcttcagatgaatgcagggaaattggctggaaaatacagccattacaaaaacaaatgcctaaacttcaagattgatagagaaaggctgatgaatgatgttaggatcagtgaaagagagagcaattctgaggcattaggtaaagcactgtcattgacaaattgtacaactgcagttctaaagaacctatgtttttacagtcaagaatccccacagtcatacacatccttgggtcctgatactggaaggctcaagttttccttatcttacaaagaacaagttggagggaacagggaactttatataggtgacctgaggacaaaaatgttcacacgcctaattgaggattattttgaggcactaactaagcaatatagagggagctgtcttaataatgaaaaggaattccacaatgccattctagccatgaaattgaatgtttcactaggtcaggtctcttatagcctcgatcacagcaagtgggggcctatgatgtccccttttcttttcctggtgtttcttcaaaatttgcgatgggagacaagagatgatatagaggacataaaaagtaaggattacgtgtccactttgctgtcgtggcacattcacaagttaattgaggtacctttcaatgttgtgaatgcaatgatgagatcttatcttaagtctaggttaggtttgaaaaaatcactccaccaaacgtcaacagaagctttcttctttgaatactttaaacaaaacagaataccatcacatctcagctcaataattgacatggggcaagggatcttgcacaatgcttctgacttctacggtctagtgagtgagagattcataaattattgcattaagtgtctatttgaagatgaagttgattcatatacctctagtgatgatcaaatatcactatttggcaaggatctttcagatttactctcaaatgagcctgaggaattccaagccattctagaatttcactatttcctaagtgatcaattgaataaattcatcagtccaaagagtgttattggttcatttgttgctgagttcaaatcaaggttttatgtctggggtgatgaagttccattgttaacgaaattcgtggctgccgccctccacaacgttaagtgtaaggagccacatcaattagctgaaactattgacactatcattgatcagtcagtggccaatggtgtgcctgtcacactatgtaacgctattcaggagagaacactgaatctacttagatatgcacaatatcccattgatcctttcttgttgtttttggattctgatgttaaagattgggttgatggcaataggggctataggattatgaggaacattgaggcaatcctaccagaaagcactcagaaagttaggaaggtcctaaggacagtttttaataagctgaaattaggagagcttcatgaagaattcacagccatctacttgtcaggagaccccgcagattccttcaagaaacttaccagccttgttggtgatgacaccctctcagaagaggatttatcggtgtgttggcttaatttgacaactcatcaccctttaaagatggtcatgagacagaaggtcatttacacaggtgctgttgaactcggggaagaaaaactgcctaccttggtgaaaacattgcaaagcaagttatcctctaatttcacaagaggggcacaaaagttgctctgtgaagccgtcaacaaaagtgcctttcagagtgggatagcatcaggtttcataggtctttgcaagacactaggtagcaaatgtgttcgattctcagataggtccaccgcctatataaaatcattagtttcaagactgtcagcattggattctgtttccagcttgaaagttaagggcgtcgatctttggatcttgggtaaggagcacacaaaggcagctgaggaagcgttaggtttcttgagacctgtcctttgggattacttctgcatagccttatctacatcacttgagctgggttcctgggtgttgggtgaacccaaagtgaaggagaaaacatcctcaattcccttcaagccatgtgactatttcccaatgaagcccactaccacaaaactcttggaagacaaggtggggtttaaccatattattcactcattcagaagactttacccatctctatttgagagacacctcttgcccttcatgagtgacctagcatcaacgaaaatgaggtggacaccaaggattaagtttcttgatctttgtgtggttctagatgtgaattgtgaggcaatgtcattaatttctcatgttgtcaagtggaagagagaagagcattatgtggttctgtcttcagatttagcaatagcacatgagaggtctcatctcccaatcacggatgaaagggtggtgaccacttatgatgtggtacaaaatttcctgagacaaatctactttgagtccttcatcagaccattcgttgcaacaagcaggactttaggttcttttacttggtttccacatagatcttcaattcctgagtcggaagggcttgacaacctcggccccttttcttcttttatagaaaaggttatttataagggtgttgaaagacccatgtacaggcatgatctttactcaggttatgcttggctggactttgaatgtgcaccagcaattctaaacttaggacagctcatagcatcaggattaaccgagcagcacgtctttgagtcggtaagtgagctgcttgaagcttttgccgacctcagtgttgggagcgttcaaatttctgtcacagtaaattttcaggtgagaagtcagggtgaatcattgaaagagaaatttagtctccacctccttttcaaaggggtggtgttggaaggtggattattcaagcctcattcccttgatgtaacttacagtggtagtgttcaaagatccgcaattaaagattgctggagagttgcacagacatctacatggtttaaaagggaaaccacatcaatttggttgctgtccactgaaaatatttgtgactacttgagggatagttcccccattcctgatgtgatacccttgtccgtcttattgaatgaggagatcctggacctggaggaacatgatttcacgcatatagggcctgagcatgttgaaatccccttagttgttgactcaggataccttattgaagggaccaggaaactcctgcccttcaaccccaacatccatgaccaggatcttaatgtttttattggtgagctaatggaggatcattccgaaatcttggagagatctttgagcaagatgctgagatccagaatggaccaaggactacactggctacaacttgatattataggggttgtgggacgatgcatgcctgaaggctacgaaaacttccttactagagtgttctccggaattgacttctgggcagattttaaaggctatagtctctgctacagtagatcgcaggcttcactgatgatccagtcttcagaggggaagtttagattaagagggaggctgtgcaggcccctctttgaagaggtggggcctcccctcgacattgagtaggtgctccgagaacagctcgcgcatctccccccggggggagccccggcggggggtccccccgggggggaggggaggggtgtttgggaggggcttcggggcggagttctgcctcaggggctgtagggtgggggattttgggatggtgggatttctggtggtgctgtcggctgggtctggagctctagtctgaagggtagtttgtgtttgcagatggggcacctatctgacactgtgtgtaaaagtgtaagacagttcaaacataggtagtgatcgttgcacctgactaaccccctcctttcgaaccagcagctcttgcagaattcagggccagttcccctggcatctggtattactggatgccttggttctgtctccttgaggagatttgtgctgggctgtcccttggactgcgttttccccatgactcttgccggatgcggctttgagtgcagaatgaagtatcagcaaagatccacaaaattgcctaggatccccggtgcg

SEQ ID NO: 15 - DNA, 3398 bp, synthetic construct
  misc_feature 1..3398: note = MOPEVAC S GPC Guanarito (INH-95551)
  misc_feature 1..68: note = 5'NC region
  misc_feature 69..1782: note = Nucleoprotein "NP" ORF
  misc_feature 1237..1239: note = possible mutated residue : D390
  misc_feature 1243..1245: note = possible mutated residue : E392
  misc_feature 1246..1248: note = possible mutated residue : G393
  misc_feature 1357..1359: note = possible mutated residue : H430
  misc_feature 1468..1470: note = possible mutated residue : D467
  misc_feature 1654..1656: note = possible mutated residue : H529
  misc_feature 1669..1671: note = possible mutated residue : D534
  misc_feature 1783..1905: note = Intergenic Region (IGR)
  misc_feature 1906..3345: note = GPC Guanarito (INH-95551) ORF
  misc_feature 3346..3398: note = 3' NC region
  source 1..3398: mol_type = other DNA
gcgcacagtggatcctaggctaattgattgcgcctttggaatcagctctgcaccgaaaagcctccaacaatgtccaattcaaaggaggtgaagtccttcttgtggacacagagcctgaggagagaactctcagggtactgctccaacataaagatccaagtcatcaaggatgctcaagcacttcttcatgggctggacttctctgaagttgccaatgttcaaaggttgatgagaaaggagaagagggatgactctgacctgaaaagattgagggacctaaaccaggcagtgaacaatctagttgagttaaagtcagtccaacagaagaatgttttgagagtggggacactaacctctgatgacctcctcgtccttgctgccgacctggacagactcaaagcaaaagtcatcagaggtgagaggcctcttgctgctggagtctatatgggcaacctaacagctcagcagctagaacagaggagggttttgttacagatggtcggaatgggtggcgggttccgggcaggaaacactctcggagatggcattgttagagtgtgggatgttcgaaacccagagcttttaaacaatcagtttgggacaatgccaagcctgacgattgcttgcatgtgcaaacaagggcaggcagatctgaatgatgtgatccaatcgttgtcagacttggggcttgtgtacactgcaaagtatccaaacatgtctgacttagacaaactctctcagacccacccaatcttggggatcattgagcccaagaaaagtgccataaacatatcagggtacaattttagcctgtcagctgcggtgaaagctggtgcttgtctaatagacggcggaaacatgctggagaccatcaaagtaacaaaatccaatttggaaggaattttgaaggctgccttgaaagtcaagcgttctttgggaatgtttgtctctgacacgccaggggaaaggaacccttatgaaaatctcctctacaaactatgtctttctggtgagggttggccttacatagcatcaagaacatcgatcgtcggcagggcttgggataacacaactgttgatctgagtggtgatgtgcaacagaatgcaaagcctgacaaaggtaactccaacagactcgctcaggcccaaggcatgcctgctggtttgacctactctcagacaatggaactcaaagacagcatgttgcaattggatccaaatgctaagacatggattgccatagcagcgagacctgaagaccccgtggagatagctatctatcaacctaataatggtcagtatattcatttttacagggaaccaacagacattaaacaattcaaacaagactccaaagcctctcatggcattgacatccaagacctattctcagttcagccggggttgacaagtgctgtaattgagagcctgccaaagaacatggtcttgtcgtgtcaaggtgctgatgccatcagaaagcttcttgactcccagaacaggagggacataaaactgattgatgtgtccatgcagaaagacgatgcaagaaaatttgaggataagatctgggatgaatacaaacacctttgtagaatgcatacggggattgtaacgcaaaagaagaagagaggtggcaaagaagaggtgacaccacactgtgcattgctggcttgtctcatgtttgaagcagcagtcatagggagtccacaaattccaacccccagaccagtcttgagtagagacctggtgtttagaacaggtcctcccagagttgtcctgtaagaagggacacctccacgacccaccgcctctggcggtgggtcgtggaggccatcaaggaacagtcactccaggcccccgagacccaccgccgaaggcggtgggtctcgggggtgcccctgttcttcaatgctttctcttccaggttacttgttttccaagtttttgaaacctgccacacctgcaagcaccatttctatcgagcctgtgaggcaaagggcacgggtcaccctgtatatgtctatgtgttgggaacccaaccaggtgtaaaaagagacttgtggtgaaaaagattgcactccagaaacatagatcaaccagtgttaggggagtcttcccctgcctatcttggtattctttgcttaacatctctgctattaggtgatcactctccaagatccactcatttctaaaatcactctcattcaaataggaaccattactgaccagccaacaccgaggcagtgagtgctcaccagatttcgtgtggtttatgtaccagaatcttgtgtagttgcaatatgggaccttcaaaatctccttcagtttgtttctcatcaataaattatcagatattagactatttattgagtgagtcaacatgttgacagcagttttagtttggttgttcagtttttcaatggcattcttattgaaatcaaacagcctcaacatgtcacagaactcagaatcatgattcaaattacacttggcgacagctgtgttgccaaagcacttcaaatccccagcaaccaacatccacctttcaagacagtaaccacctggcatgtcattaccctttgggtcagaaagtgaccaactaaaaaatgccagtggttttcttccaacaaggtaacctgcatttttaacaagaaatctcagtgtattcacatgactatttgagcattgaccttcccaggtagtgttcctcataaccacagacatttttgaatagctatcattagttatgtagttcccgaataaatgtcggatgcctacctcaatcttgctcctatagtgatgggtctcaggactatctgttaaattcaattggaatgtaaattcagcattagttgtctttttaggctcacagcagattcttggatccctcaaaaagtcatgtcccaatcccttaattgtccacccaagaatccaatcaaactctggagagccttttatacattgagtgtagttgtcgagtatggttggactggacgaacagaggacagggtgcacatccacatactcaactgataatgcctgattactgttatgagaaagcctaattagactgtaagagttgttgaccctacatagtgaaggtaattcatggaagacacctcctagcttaactgtaaatgactcaaaatttgtatgatgaccaactttgaaagagcatgaccttcccgccagaaccaagaacacaaacagctgaagaattcctgatttccaaacattcacaatgccctttataatagcaagcagtgtaaccacagccaaggctacattgagtgcttcttcaaagaagatcggtatgtcctgaaagaaactgattaactgtcccatctccgagatgctctagaaatgcgcaaccaaaaagcctaggatccccggtgcgc

SEQ ID NO: 16 - DNA, 3449 bp, synthetic construct
  misc_feature 1..3449: note = MOPEVAC S GPC Machupo (Carvallo)
  misc_feature 1..68: note = 5'NC region
  misc_feature 69..1782: note = Nucleoprotein "NP" ORF
  misc_feature 1237..1239: note = possible mutated residue : D390
  misc_feature 1243..1245: note = possible mutated residue : E392
  misc_feature 1246..1248: note = possible mutated residue : G393
  misc_feature 1357..1359: note = possible mutated residue : H430
  misc_feature 1468..1470: note = possible mutated residue : D467
  misc_feature 1654..1656: note = possible mutated residue : H529
  misc_feature 1669..1671: note = possible mutated residue : D534
  misc_feature 1783..1905: note = Intergenic Region (IGR)
  misc_feature 1906..3396: note = GPC ORF
  misc_feature 3396..3449: note = 3' NC region
  source 1..3449: mol_type = other DNA
gcgcacagtggatcctaggctaattgattgcgcctttggaatcagctctgcaccgaaaagcctccaacaatgtccaattcaaaggaggtgaagtccttcttgtggacacagagcctgaggagagaactctcagggtactgctccaacataaagatccaagtcatcaaggatgctcaagcacttcttcatgggctggacttctctgaagttgccaatgttcaaaggttgatgagaaaggagaagagggatgactctgacctgaaaagattgagggacctaaaccaggcagtgaacaatctagttgagttaaagtcagtccaacagaagaatgttttgagagtggggacactaacctctgatgacctcctcgtccttgctgccgacctggacagactcaaagcaaaagtcatcagaggtgagaggcctcttgctgctggagtctatatgggcaacctaacagctcagcagctagaacagaggagggttttgttacagatggtcggaatgggtggcgggttccgggcaggaaacactctcggagatggcattgttagagtgtgggatgttcgaaacccagagcttttaaacaatcagtttgggacaatgccaagcctgacgattgcttgcatgtgcaaacaagggcaggcagatctgaatgatgtgatccaatcgttgtcagacttggggcttgtgtacactgcaaagtatccaaacatgtctgacttagacaaactctctcagacccacccaatcttggggatcattgagcccaagaaaagtgccataaacatatcagggtacaattttagcctgtcagctgcggtgaaagctggtgcttgtctaatagacggcggaaacatgctggagaccatcaaagtaacaaaatccaatttggaaggaattttgaaggctgccttgaaagtcaagcgttctttgggaatgtttgtctctgacacgccaggggaaaggaacccttatgaaaatctcctctacaaactatgtctttctggtgagggttggccttacatagcatcaagaacatcgatcgtcggcagggcttgggataacacaactgttgatctgagtggtgatgtgcaacagaatgcaaagcctgacaaaggtaactccaacagactcgctcaggcccaaggcatgcctgctggtttgacctactctcagacaatggaactcaaagacagcatgttgcaattggatccaaatgctaagacatggattgccatagcagcgagacctgaagaccccgtggagatagctatctatcaacctaataatggtcagtatattcatttttacagggaaccaacagacattaaacaattcaaacaagactccaaagcctctcatggcattgacatccaagacctattctcagttcagccggggttgacaagtgctgtaattgagagcctgccaaagaacatggtcttgtcgtgtcaaggtgctgatgccatcagaaagcttcttgactcccagaacaggagggacataaaactgattgatgtgtccatgcagaaagacgatgcaagaaaatttgaggataagatctgggatgaatacaaacacctttgtagaatgcatacggggattgtaacgcaaaagaagaagagaggtggcaaagaagaggtgacaccacactgtgcattgctggcttgtctcatgtttgaagcagcagtcatagggagtccacaaattccaacccccagaccagtcttgagtagagacctggtgtttagaacaggtcctcccagagttgtcctgtaagaagggacacctccacgacccaccgcctctggcggtgggtcgtggaggccatcaaggaacagtcactccaggcccccgagacccaccgccgaaggcggtgggtctcgggggtgcccctgttcttcaatgtcttttgtgccagatggtgggtttcttcaatctgggatatttgccacatctacaacctccgaagctgtccagcttatgaggcaaaggacaggcttcgcctttgaggtgtcgatgggtgggtattccgactagatgaaggaacaatgatgctgtgaagaaaattgtgctccagaaacaaatatcaactaatgtgattggggttttgccttgcctttcagcatattccttacttaacatctctgagatgaggtgatcactctctaaaatccagtcattcctgaattcagaagtgttaagataacttccattccttatcaaccaacatcttggaagagtgtgctgccctgtcagggtatgattgacataccaaaactttgtgtaattacaataagggatgctcattagctctttaattttattcttcattaacaaattatctgagattaaggcattcactgtctggcttagaagattgatttctttctttgattcatcattgagggtctttattgcattcttgttatagtcgaatagcctcagcatatcacagaactctgagtcatgattttgattacatttagcaacagcagtgtttccgaaacatttcattttggctgctatcaacatccattcctctagacaataacctcctggcatgtcctttcctgaggagtctgtcagagaccatgagaaaaatgctttcaaagacctctcaaagttgagatgtgtcttacttctcacaaggaaatgaagggtgttcacatggtcaaactgacatttggaaagatgattcacgccacagtagtcaaaggaactggggtcaccacactgattgatggtcaggtagcacactttcccttcctcacacgggtcatggaagcccctgaagagatgcctcataccatttcttatcttctttccatacactctggcatcatcagctttgctgatgttgaattgaatgttagatccctccttctttgtcttgtttctacatagcattggaggatccataagccagtcatgtcccaaagcatttgagaaccaatgaatagcccagcttgagtcagacttgttaagacaatttccaaggtcttctggttcatagattgatacatcatactccttcatgaggactgaaatatcagaaacacgaatcaggaaggtgttcacacctcccctcatataataaaaactattgttaagcatgcagagagatgggagctcatttgaatggttagctaaaagtctctgcatggtaagggtgactgactggaactcagtgtgtaggcctattttgaatgtgccatccgagcaggacctccctgctaggaggagaaagaagatgaactggaagagaccacttttgtaaaggttaatgatgcctttgatgacagctatgagactaacagccactaaagcgatgttcagagcttcctgtagaaaaacaggaatctcctgaaagaagctgataagctgccccatctccgagatgctctagaaatgcgcaaccaaaaagcctaggatccccggtgcgc

SEQ ID NO: 17 - DNA, 3416 bp, synthetic construct
  misc_feature 1..3416: note = MOPEVAC S GPC Chapare
  misc_feature 1..68: note = 5'NC region
  misc_feature 69..1782: note = Nucleoprotein "NP" ORF
  misc_feature 1237..1239: note = possible mutated residue : D390
  misc_feature 1243..1245: note = possible mutated residue : E392
  misc_feature 1246..1248: note = possible mutated residue : G393
  misc_feature 1357..1359: note = possible mutated residue : H430
  misc_feature 1468..1470: note = possible mutated residue : D467
  misc_feature 1654..1656: note = possible mutated residue : H529
  misc_feature 1669..1671: note = possible mutated residue : D534
  misc_feature 1783..1905: note = Intergenic Region (IGR)
  misc_feature 1906..3363: note = GPC ORF
  misc_feature 3363..3416: note = 3' NC region
  source 1..3416: mol_type = other DNA
gcgcacagtggatcctaggctaattgattgcgcctttggaatcagctctgcaccgaaaagcctccaacaatgtccaattcaaaggaggtgaagtccttcttgtggacacagagcctgaggagagaactctcagggtactgctccaacataaagatccaagtcatcaaggatgctcaagcacttcttcatgggctggacttctctgaagttgccaatgttcaaaggttgatgagaaaggagaagagggatgactctgacctgaaaagattgagggacctaaaccaggcagtgaacaatctagttgagttaaagtcagtccaacagaagaatgttttgagagtggggacactaacctctgatgacctcctcgtccttgctgccgacctggacagactcaaagcaaaagtcatcagaggtgagaggcctcttgctgctggagtctatatgggcaacctaacagctcagcagctagaacagaggagggttttgttacagatggtcggaatgggtggcgggttccgggcaggaaacactctcggagatggcattgttagagtgtgggatgttcgaaacccagagcttttaaacaatcagtttgggacaatgccaagcctgacgattgcttgcatgtgcaaacaagggcaggcagatctgaatgatgtgatccaatcgttgtcagacttggggcttgtgtacactgcaaagtatccaaacatgtctgacttagacaaactctctcagacccacccaatcttggggatcattgagcccaagaaaagtgccataaacatatcagggtacaattttagcctgtcagctgcggtgaaagctggtgcttgtctaatagacggcggaaacatgctggagaccatcaaagtaacaaaatccaatttggaaggaattttgaaggctgccttgaaagtcaagcgttctttgggaatgtttgtctctgacacgccaggggaaaggaacccttatgaaaatctcctctacaaactatgtctttctggtgagggttggccttacatagcatcaagaacatcgatcgtcggcagggcttgggataacacaactgttgatctgagtggtgatgtgcaacagaatgcaaagcctgacaaaggtaactccaacagactcgctcaggcccaaggcatgcctgctggtttgacctactctcagacaatggaactcaaagacagcatgttgcaattggatccaaatgctaagacatggattgccatagcagcgagacctgaagaccccgtggagatagctatctatcaacctaataatggtcagtatattcatttttacagggaaccaacagacattaaacaattcaaacaagactccaaagcctctcatggcattgacatccaagacctattctcagttcagccggggttgacaagtgctgtaattgagagcctgccaaagaacatggtcttgtcgtgtcaaggtgctgatgccatcagaaagcttcttgactcccagaacaggagggacataaaactgattgatgtgtccatgcagaaagacgatgcaagaaaatttgaggataagatctgggatgaatacaaacacctttgtagaatgcatacggggattgtaacgcaaaagaagaagagaggtggcaaagaagaggtgacaccacactgtgcattgctggcttgtctcatgtttgaagcagcagtcatagggagtccacaaattccaacccccagaccagtcttgagtagagacctggtgtttagaacaggtcctcccagagttgtcctgtaagaagggacacctccacgacccaccgcctctggcggtgggtcgtggaggccatcaaggaacagtcactccaggcccccgagacccaccgccgaaggcggtgggtctcgggggtgcccctgttcttcatcagtgtttcctgtgccaggttgttggtttcttgagttctggatatctgccacacctgcatccaccattgctgttgagcttgtgaggcagtgggcaaggttctccctgaatgtgtctgtgagttgggaagcctactagatggagaaacaatgttgttgtgaagaacagtgtgctccaaaagcagatgtcaacaagtgttattggggttttcccttgtctgtcaaaatactctttgttcaacatttcggacaacaagtggtcactctcaagaatccaatcatttctaaattcactttcattgaggtagctgttgtttttaaccatccaacagcgtggaagtgaatgctctcctgtgatggtgtggttgacataccagaattttgtgtagttgcagtagggagtgtccaataactcttttaatctattcttcatcaaaagattgtcagaaattaaagcattaatggaatgtgttaatagattgactttgttctttgtgttgtcattcaatgactcaattgcttttttgttaaattcaaacagtttcaacatgtcacagaactcagagtcatgatttaagttacactttgcaagagcagtgttgccaaaacatttcaggtcagatgtgactaacatccacctttccaaacaataacctccaggcatgtcgttgccagcagcatctgtgatcgtccaagtgaacaccccttgcaatcttcttaatttaacagcatgcccagcattagccatcatcaaatgcaacgtgttgacatgattggacttgcactgattcacccaagtctgatttctgataactatgaactgcttaggtgttttatcaaacatcaattttgtgccaaatagtatggccagcctttggagcatggtttctttgaaatcatgagaacctgcactatcgctcacatttatctgaatgcgtgtttcattgacagatgatgtcactttttcacacaagaggggtggataatttagaaaatcatatttcagagctctcaaaatccactccaatagacctagaagttctgatctatgtttaatgtctgggtgaacacagtttgataggttgcccattacacgtggatcatgtggcgagagcaagacagacatgtccagtgcagcaatctctacacaccatgttgcattctcactttcacggatgtaataaagagttttgttcactgtgcaagaaataggatggtcctcaaagacctttaacatgtttattgtgatgttttggagttctgtggatcttccaattttgaatgagcaggaccttccagctagaataaggaagaccaggagttgaaacaaaccacttttccacagattgaccaacccttttaggatagcaatgaggctgacagctattagagcaatgttgattgcttcctgtatgatgttaggaatttcctgaaagaaactcacaagttgacccatctccgagatgctctagaaatgcgcaaccaaaaagcctaggatccccggtgcgc

SEQ ID NO: 18 - DNA, 3425 bp, synthetic construct
  misc_feature 1..3425: note = MOPEVAC S GPC Sabia
  misc_feature 1..68: note = 5'NC region
  misc_feature 69..1782: note = Nucleoprotein "NP" ORF
  misc_feature 1237..1239: note = possible mutated residue : D390
  misc_feature 1243..1245: note = possible mutated residue : E392
  misc_feature 1246..1248: note = possible mutated residue : G393
  misc_feature 1357..1359: note = possible mutated residue : H430
  misc_feature 1468..1470: note = possible mutated residue : D467
  misc_feature 1654..1656: note = possible mutated residue : H529
  misc_feature 1669..1671: note = possible mutated residue : D534
  misc_feature 1783..1905: note = Intergenic Region (IGR)
  misc_feature 1906..3372: note = GPC ORF
  misc_feature 3372..3425: note = 3' NC region
  source 1..3425: mol_type = other DNA
gcgcacagtggatcctaggctaattgattgcgcctttggaatcagctctgcaccgaaaagcctccaacaatgtccaattcaaaggaggtgaagtccttcttgtggacacagagcctgaggagagaactctcagggtactgctccaacataaagatccaagtcatcaaggatgctcaagcacttcttcatgggctggacttctctgaagttgccaatgttcaaaggttgatgagaaaggagaagagggatgactctgacctgaaaagattgagggacctaaaccaggcagtgaacaatctagttgagttaaagtcagtccaacagaagaatgttttgagagtggggacactaacctctgatgacctcctcgtccttgctgccgacctggacagactcaaagcaaaagtcatcagaggtgagaggcctcttgctgctggagtctatatgggcaacctaacagctcagcagctagaacagaggagggttttgttacagatggtcggaatgggtggcgggttccgggcaggaaacactctcggagatggcattgttagagtgtgggatgttcgaaacccagagcttttaaacaatcagtttgggacaatgccaagcctgacgattgcttgcatgtgcaaacaagggcaggcagatctgaatgatgtgatccaatcgttgtcagacttggggcttgtgtacactgcaaagtatccaaacatgtctgacttagacaaactctctcagacccacccaatcttggggatcattgagcccaagaaaagtgccataaacatatcagggtacaattttagcctgtcagctgcggtgaaagctggtgcttgtctaatagacggcggaaacatgctggagaccatcaaagtaacaaaatccaatttggaaggaattttgaaggctgccttgaaagtcaagcgttctttgggaatgtttgtctctgacacgccaggggaaaggaacccttatgaaaatctcctctacaaactatgtctttctggtgagggttggccttacatagcatcaagaacatcgatcgtcggcagggcttgggataacacaactgttgatctgagtggtgatgtgcaacagaatgcaaagcctgacaaaggtaactccaacagactcgctcaggcccaaggcatgcctgctggtttgacctactctcagacaatggaactcaaagacagcatgttgcaattggatccaaatgctaagacatggattgccatagcagcgagacctgaagaccccgtggagatagctatctatcaacctaataatggtcagtatattcatttttacagggaaccaacagacattaaacaattcaaacaagactccaaagcctctcatggcattgacatccaagacctattctcagttcagccggggttgacaagtgctgtaattgagagcctgccaaagaacatggtcttgtcgtgtcaaggtgctgatgccatcagaaagcttcttgactcccagaacaggagggacataaaactgattgatgtgtccatgcagaaagacgatgcaagaaaatttgaggataagatctgggatgaatacaaacacctttgtagaatgcatacggggattgtaacgcaaaagaagaagagaggtggcaaagaagaggtgacaccacactgtgcattgctggcttgtctcatgtttgaagcagcagtcatagggagtccacaaattccaacccccagaccagtcttgagtagagacctggtgtttagaacaggtcctcccagagttgtcctgtaagaagggacacctccacgacccaccgcctctggcggtgggtcgtggaggccatcaaggaacagtcactccaggcccccgagacccaccgccgaaggcggtgggtctcgggggtgcccctgttcttcagtggttcttgtgccaggtgattggcttttttagttcagggtatttcccacatctacatcctcctctactgttgagcctatggggtagtgggcagggttcaccacgtatgtgtctatgagttggaaagcctaccaggtgaagaaacagtgttgttgtgaaaaacaaagtgctccagaaacagatatccaccaaagtcaacggtgtctttccctgtctatctatgtattctttattgagcatttcagacaataaatgatcactctcaataatccaatcatttctaaattcactttcattcaagtagctattatttctaacaagccagcaccgtggcaatgagtgttcccctgatgctgtgtgattgacataccaaaatttggtgtaattacaataaggagtgttcaacaattctttaagtcgattcttcatcagtaagttgtcagatattaatgcattaattgagtgggtcagcaagtttaccttgttttttgtattgtcattcaatgtctctatcgcttttttgttgaactcaaacaatttcaacatgtcacagaattccgaatcgtggtcaaggttacattttgctagtgctgtgtttccaaagcacttaagatctgacgtcactagcatccatctttcaagacaataaccaccaggcatgtcattgcccactgcatcagttattgtccaggagaaaatgccgagtggtcttctagaaccagaagagcgaccagcattcgccaacattaaatgcatggagtttacatgattcatttcgcattgatttttccaagttgaatttctaatcaataaaaacctttttttacctaaatcctggatatttgaaaatgcaattcttgaaccgaatagaacccctagtctttgaaggatggtatcttcaaacccgtgagacccaaacccctcagtgatgtttatctgcacacgtgtttcattcactgttgaagtttgcttttgacacaacggtgttggatcatgattgaagtcatactttagggctctaaaaatccactcaagtaagccaaccatcctgcttctgtgctcaactgcagggtgcacacagtttgacagattgttgaggacttgacgatcatgttcagccatgagcagggtaacatcagtcacagacacctcaagacaccaagtggcatttttgttttcatggacatagtaggtggaatgattcaccatgcaagatgtggggtggtcctcgaataccttcaacatatcaaacgttatgttttgcaattctgtgctccttccaattctaaaagaacacgatcttcctgctagagtcaaaaagaatatcaattggaaaaggccactcttccacaagttaatcatccctttcaaggcagcaattaagctcactgctatcagagctatgttgatagcctcatggatgatattcggaacttcttcaaaaaagctgaacaattgacccatctccgagatgctctagaaatgcgcaaccaaaaagcctaggatccccggtgcgc

SEQ ID NO: 19 - DNA, 3416 bp, synthetic construct
  misc_feature 1..3416: note = MOPEVAC S GPC Junin
  misc_feature 1..68: note = 5'NC region
  misc_feature 69..1782: note = Nucleoprotein "NP" ORF
  misc_feature 1237..1239: note = possible mutated residue : D390
  misc_feature 1243..1245: note = possible mutated residue : E392
  misc_feature 1246..1248: note = possible mutated residue : G393
  misc_feature 1357..1359: note = possible mutated residue : H430
  misc_feature 1468..1470: note = possible mutated residue : D467
  misc_feature 1654..1656: note = possible mutated residue : H529
  misc_feature 1669..1671: note = possible mutated residue : D534
  misc_feature 1783..1905: note = Intergenic Region (IGR)
  misc_feature 1906..3363: note = GPC ORF
  misc_feature 3363..3416: note = 3' NC region
  source 1..3416: mol_type = other DNA
gcgcacagtggatcctaggctaattgattgcgcctttggaatcagctctgcaccgaaaagcctccaacaatgtccaattcaaaggaggtgaagtccttcttgtggacacagagcctgaggagagaactctcagggtactgctccaacataaagatccaagtcatcaaggatgctcaagcacttcttcatgggctggacttctctgaagttgccaatgttcaaaggttgatgagaaaggagaagagggatgactctgacctgaaaagattgagggacctaaaccaggcagtgaacaatctagttgagttaaagtcagtccaacagaagaatgttttgagagtggggacactaacctctgatgacctcctcgtccttgctgccgacctggacagactcaaagcaaaagtcatcagaggtgagaggcctcttgctgctggagtctatatgggcaacctaacagctcagcagctagaacagaggagggttttgttacagatggtcggaatgggtggcgggttccgggcaggaaacactctcggagatggcattgttagagtgtgggatgttcgaaacccagagcttttaaacaatcagtttgggacaatgccaagcctgacgattgcttgcatgtgcaaacaagggcaggcagatctgaatgatgtgatccaatcgttgtcagacttggggcttgtgtacactgcaaagtatccaaacatgtctgacttagacaaactctctcagacccacccaatcttggggatcattgagcccaagaaaagtgccataaacatatcagggtacaattttagcctgtcagctgcggtgaaagctggtgcttgtctaatagacggcggaaacatgctggagaccatcaaagtaacaaaatccaatttggaaggaattttgaaggctgccttgaaagtcaagcgttctttgggaatgtttgtctctgacacgccaggggaaaggaacccttatgaaaatctcctctacaaactatgtctttctggtgagggttggccttacatagcatcaagaacatcgatcgtcggcagggcttgggataacacaactgttgatctgagtggtgatgtgcaacagaatgcaaagcctgacaaaggtaactccaacagactcgctcaggcccaaggcatgcctgctggtttgacctactctcagacaatggaactcaaagacagcatgttgcaattggatccaaatgctaagacatggattgccatagcagcgagacctgaagaccccgtggagatagctatctatcaacctaataatggtcagtatattcatttttacagggaaccaacagacattaaacaattcaaacaagactccaaagcctctcatggcattgacatccaagacctattctcagttcagccggggttgacaagtgctgtaattgagagcctgccaaagaacatggtcttgtcgtgtcaaggtgctgatgccatcagaaagcttcttgactcccagaacaggagggacataaaactgattgatgtgtccatgcagaaagacgatgcaagaaaatttgaggataagatctgggatgaatacaaacacctttgtagaatgcatacggggattgtaacgcaaaagaagaagagaggtggcaaagaagaggtgacaccacactgtgcattgctggcttgtctcatgtttgaagcagcagtcatagggagtccacaaattccaacccccagaccagtcttgagtagagacctggtgtttagaacaggtcctcccagagttgtcctgtaagaagggacacctccacgacccaccgcctctggcggtgggtcgtggaggccatcaaggaacagtcactccaggcccccgagacccaccgccgaaggcggtgggtctcgggggtgcccctgttcttcagtgtcttctacgccaaactgttggtttctttagattggggtacttaccacatctgcaaccacccaagctgttcaacctgtgtggcaaagggcatgcttcgcccctgatgtgtctgtgggtaggtatgcccaccaaatgaaggaagagtgacgctgtgaaaaatactgtgctccaaaaacagatgtcaactaaagtcaaaggagttttaccctgtctgtccgaatactctttgcttagcatttcagaaattaagaagtcactctccaatatccagtcattacggaagtcagagatgttcaaatagctgttgttttttattaaccagcaccttggtaatgagtgttgtcctgaaagtgtgtggttgacataccaaaattttgtgtaattgcagtaggggacactcatcagttccctaattttgtttttcatcaataaattgtcagatatcaaggcattgattgtctgccccatcagatttacttgtttctttgtttcatcatttagggttttgatggcatttttgttgtaatcaaaaagcctcaacatgtcacagaattcagagtcatgattcaaattgcattttgctacagcagtattgccaaaacacttcattttggctgccacgagcatccactcttccagacaatagcctccaggggtatccttgccggatgagtctgtcaaagaccaggagaagaatgctttcaaggacctccttggaagttgaatatttttgcctcttgtaaggaaatgtaatgtgttaacatggtcgagtggacattggagaggccaactggtgggttgtgccttcattaaacacagtttgccattcaagcaagagtcaggatattctctatataaatgatgcatgccagtcttaaacttcttagcatagttttcattaacaccagtcttggaggtgttgacttgaaagatgaagccttctgtctttgtacggttcctacacagaaatggtgggtctagatgccaatcatgtcccacagcattcatgaaccattgagacaaccaaatttgatcatcacttttagaacaccagctcatatctgccggatgttgtattataacatcatactgtggcaacagtacttcaatatcatcaaagctgatcataaatgaagcattgccccccttaatgtaaagatggctcttgtttaaggtacacaacaaaggtaggtcatgtggattgttggaaaagagacccaccattgagaaggacacagtctggaactcagtatgcagtccgattttgaaagcttcttctgtgcaggatcttcctgcaagcgctaggaatacaaagaattggaataaaccacttttgtacaagttcactatccccttaatgatggcaatgagactgactgctacaagagcaatgttcagagcctcctgcaaaaaagttggtatttcttgcatgaagctaatgaactgccccatctccgagatgctctagaaatgcgcaaccaaaaagcctaggatccccggtgcgc

SEQ ID NO: 20 - DNA, 6714 bp, synthetic construct
  misc_feature 1..6714: note = MOPEIA RNA dependant RNA polymerase "Lpol" Accession number AEO89358.1
  source 1..6714: mol_type = other DNA
atggaggagttgctgtccgagagcaaagatcttgtgagcaggtacctcttagaggatgagaggctttcaaaacagaagctagccttcctagttcagacagagccgagaatgctattaattgagggccttaagttgttgtcactctgtattgagatagacagttgcaaagccaatggttgtgagcacaactcagaagacctttctgttgaaatccttctgcagagacaaggtgttctgtgccctggtctaccatttgtggtgccagatggattcaaattcagtgggaacacattaatattactggaatgctttgtcaggacttcaccaataaactttgagcaaaagtataaagaagataccattaagctagagtctttaaagcctgacttatcatctgttgacatcattctgttgcccttgatagacggcagaaccaatttctacactgatttgtttccagaatgggccaatgaaaggtttaggcatatcctatttagcttacttgaattctcgcagcagtcatcaaaaatgtttgaagaatctgagtactcaaggctttgtgaatctttaacaaaagcaggagtgagaacatcgggcattgagagcctcaatgttttgacagattctcgatctgatcattatgagagggtgttggagctctgccacagaggcatcaataacaaaatgtccattcttgatgtgaaaaaagagattgtatcagaatttcatgcttttaggaataagcttaaagagggtgagatagaaagacaatttgtcaggacagataggcgacagctcttgagagatttcaacaacctttatattgacagagagggggacacaccctcagagattgatcctctaaaagagaggtttgtaaaatcctcgcctatggtaacagcgctctatggtgactacgaccgttataggcaagagggagttgatcgagacagctgcttgcagaatcacttccaaagctctgtgcctggatggaagtcactgttgaataaaataaaatcactaaagttattgaacaccagaagaaaactaatgctgacttttgacgcaatcatccttttggcccacttaaaggatcttaaatgtcacggcgagctgttaggatcagaatggcttggctcctcattcttaagtgtgaacgacagattggtgtcactacaagaaacacaaaaggaccttaaaaagtggattgaaaggagaatggtgagtgcaatgaagaagaagggaggtgtaggaactctgtgtcagagatctgagcttatattctttgacatcataaacaaactcctcacaaaggccaaagaggcattatcctctgccagtttgtgctttagagattatgttaaagaggaagatatactggaggaggacagttacgagaggcttatgttaatggagaaaagagggattcagccaacaatgagctatgagaaagaagaggggaatcaattcccctaccctcttattgagttggaagctgattccatagaagacctgagaagactatctagcatctctttggcattggtgaattcaatgaagacatcatcagtagccaaagtgaggcaaaatgagtatggtgctgcaaggtacaaacgtgtacgttgtaaggaagcttttaatcaaagctttatcatgggaagcgggaatttcaacttaatttatcagaaaacaggagagtgctcaaaatgttatgccattaacaatcctgagaagggggagatttgttcattctatgcagatccaaaaaggttttttcctgcaattttctcacactgtgttatctatgagactatcaacaccatgatgagttggttgtctgaatgtatagaactcagagatcaacaaaaaactttaaaattattgctcaaaatcaccatgatcctcatacttgtgaaccctagcaaaagagcacagaagttcttgcaaggtctgcgatacttcataatggcctttgtttcagacttccaccataagcagttaatggaaaagttgagggaggatctcataacagagccggagcacctcttgtatagtgtggtgaggagcattctcaacatcatcctgggtgaaggggtgagcactatgttgactaatagatttaagtttgtgttaaacctatcatacatgtgtcattttataactaaggaaactccagataggttgacagatcagattaagtgctttgagaagtatttggagcccaagttggagtttgacagtattaacatcaacccatctgaagagggggatgaagatgaaaggatgctgctgcttgaatcagcaaacaaatttttatccaaagaaaccagtatgagtaacaacagaatatcttataaagttcctggtgtgtcaagaaaattcttctcaatgatgacgtcttcttttaacaatggctctcttttcaagaaaggagatgacctaagtgggtttaaagatccattagttactgctgggtgtgcaacagctcttgaccttgcaagcaacaaaagtgtggttgtgaataagtatactgacggagagaggatacttaattacgatcatgataaactagtggctgcttctgtttgccagctatcagaggtattccagaggaaaactaaatacctcttgagtaaggaggattatgattataaggtgcaaaaggccattagtgaccttgttgtggggaagaagtcaggttcctcaaatcccaattcacaaggggctcctgacgaattagatgagttattcttggatagttgtgcacttgactgtctagaggatgtgaagaaatctgttgatgtcgtccttgagaagtatagatatgacaggaagttccctgtgggaaatgggtcagaggagaagtccttgacagacttgaggaaggttttaggtactgaagatgtgggctgtgtttactacagactgatccaggcagagatagcacaccacatggtggaagattttgatgagtcactactacctggagatgcttatgagatgatctgcaaaggcttttttaaggatttggagttaaggtcaaagtatttctatttggattccttggactcttgcccaataacatgcatcacccaagctgtctccaccagaacattcaatgaccagcagttttttcagtgcttcaagtcactacttcttcagatgaatgcagggaaattggctggaaaatacagccattacaaaaacaaatgcttaaacttcaagattgatagagaaaggctgatgaatgatgttaggatcagtgaaagagagagcaattctgaggcattaggtaaagcactgtcattgacaaattgtacaactgcagttctaaagaacctatgtttttacagtcaagaatccccacagtcatacacatccttgggtcctgatactggaaggctcaagttttccttatcttacaaagaacaagttggagggaacagggaactttatataggtgacctgaggacaaaaatgttcacacgcctaattgaggattattttgaggcactaactaagcaatatagagggagctgtcttaataatgaaaaggaattccacaatgccattctagccatgaaattgaatgtttcactaggtcaggtctcttatagcctcgatcacagcaagtgggggcctatgatgtccccttttcttttcctggtgtttcttcaaaatttgcgatgggagacaggagatgatatagaggacataaaaagtaaggattacgtgtccactttgctgtcgtggcacattcacaagttaattgaggtacctttcaatgttgtgaatgcaatgatgagatcttatcttaagtctaggttaggtttgaaaaaatcactccaccaaacgtcaacagaagctttcttctttgaatactttaaacaaaacaggataccatcacatctcagctcaataattgacatggggcaagggatcttgcacaatgcttctgacttctacggtctagtgagtgagagattcataaattattgcattaagtgtctatttgaagatgaagttgattcatatacctctagtgatgatcaaatatcactatttggcaaggatctttcagatttactctcaaatgagcctgaggaattccaagccattctagaatttcactatttcctaagtgatcaattgaataaattcatcagtccaaagagtgttattggttcatttgttgctgagttcaaatcaaggttttatgtctggggtgatgaagttccattgttaacgaaattcgtggctgccgccctccacaacgttaagtgtaaggagccacatcaattagctgaaactattgacactatcattgatcagtcagtggccaatggtgtgcctgtcacactatgtaacgctattcaggagagaacactgaatctacttagatatgcacaatatcccattgatcctttcttgttgtttttggattctgatgttaaagattgggttgatggcaataggggctataggattatgaggaacattgaggcaatcctaccagaaagcactcagaaagttaggaaggtcctaaggacagtttttaataagctgaaattaggagagcttcatgaagaattcacagccatctacttgtcaggagaccccgcagattccttcaagaaacttaccagccttgttggtgatgacaccctctcagaagaggatttatcggtgtgttggcttaatttgacaactcatcaccctttaaagatggtcatgagacagaaggtcatttacacaggtgctgttgaactcggggaagaaaaactgcctaccttggtgaaaacattgcaaagcaagttatcctctaatttcacaagaggggcacaaaagttgctctgtgaagccgtcaacaaaagtgcctttcagagtgggatagcatcaggtttcataggtctttgcaagacactaggtagcaaatgtgttcgattctcagataggtccaccgcctatataaaatcattagtttcaagactgtcagcattggattctgtttccagcttgaaagttaagggcgtcgatctttggatcttgggtaaggagcacacaaaggcagctgaggaagcgttaggtttcttgagacctgtcctttgggattacttctgcatagccttatctacatcacttgagctgggttcctgggtgttgggtgaacccaaagtgaaggagaaaacatcctcaattcccttcaagccatgtgactatttcccaatgaagcccactaccacaaaactcttggaagacaaggtggggtttaaccatattattcactcattcagaagactttacccatctctatttgagagacacctcttgcccttcatgagtgacctagcatcaacgaaaatgaggtggacaccaaggattaagtttcttgatctttgtgtggttctagatgtgaattgtgaggcaatgtcattaatttctcatgttgtcaagtggaagagagaagagcattatgtggttctgtcttcagatttagcaatagcacatgagaggtctcatctcccaatcacggatgaaagggtggtgaccacttatgatgtggtacaaaatttcctgagacaaatctactttgagtccttcatcagaccattcgttgcaacaagcaggactttaggttcttttacttggtttccacatagatcttcaattcctgagtcggaagggcttgacaacctcggccccttttcttcttttatagaaaaggttatttataagggtgttgaaagacccatgtacaggcatgatctttactcaggttatgcttggctggattttgaatgtgcaccagcaattctaaacttaggacagctcatagcatcaggattaaccgagcagcacgtctttgagtcggtaagtgagctgcttgaagcttttgccgacctcagtgttgggagcgttcaaatttctgtcacagtaaattttcaggtgagaagtcagggtgaatcattgaaagagaaatttagtctccacctccttttcaaaggggtggtgttggaaggtggattattcaagcctcattcccttgatgtaacttacagtggtagtgttcaaagatccgcaattaaagattgctggagagttgcacagacatctacatggtttaaaagggaaaccacatcaatttggttgctgtccactgaaaatatttgtgactacttgagggatagttcccccattcctgatgtgatacccttgtccgtcttattgaatgaggagatcctggacctggaggaacatgatttcacgcatatagggcctgagcatgttgaaatccccttagttgttgactcaggataccttattgaagggaccaggaaactcctgcccttcaaccccaacatccatgaccaggatcttaatgtttttattggtgagctaatggaggatcattccgaaatcttggagagatctttgagcaagatgctgagatccagaatggaccaaggactacactggctacaacttgatattataggggttgtgggacgatgcatgcctgaaggctacgaaaacttccttactagagtgttctccggaattgacttctgggcagattttaaaggctatagtctctgctacagtagatcgcaggcttcactgatgatccagtcttcagaggggaagtttagattaagagggaggctgtgcaggcccctctttgaagaggtggggcctcccctcgacattgagtag

SEQ ID NO: 21 - DNA, 1713 bp, synthetic construct
  misc_feature 1..1713: note = MOPEIA Nucleoprotein "NP" WT Accession number AEO89356.1
  source 1..1713: mol_type = other DNA
atgtccaattcaaaggaggtgaagtccttcttgtggacacagagcctgaggagagaactctcagggtactgctccaacataaagatccaagtcatcaaggatgctcaagcacttcttcatgggctggacttctctgaagttgccaatgttcaaaggttgatgagaaaggagaagagggatgactctgacctgaaaagattgagggacctaaaccaggcagtgaacaatctagttgagttaaagtcagtccaacagaagaatgttttgagagtggggacactaacctctgatgacctcctcgtccttgctgccgacctggacagactcaaagcaaaagtcatcagaggtgagaggcctcttgctgctggagtctatatgggcaacctaacagctcagcagctagaacagaggagggttttgttacagatggtcggaatgggtggcgggttccgggcaggaaacactctcggagatggcattgttagagtgtgggatgttcgaaacccagagcttttaaacaatcagtttgggacaatgccaagcctgacgattgcttgcatgtgcaaacaagggcaggcagatctgaatgatgtgatccaatcgttgtcagacttggggcttgtgtacactgcaaagtatccaaacatgtctgacttagacaaactctctcagacccacccaatcttggggatcattgagcccaagaaaagtgccataaacatatcagggtacaattttagcctgtcagctgcggtgaaagctggtgcttgtctaatagacggcggaaacatgctggagaccatcaaagtaacaaaatccaatttggaaggaattttgaaggctgccttgaaagtcaagcgttctttgggaatgtttgtctctgacacgccaggggaaaggaacccttatgaaaatctcctctacaaactatgtctttctggagagggttggccttacatagcatcaagaacatcgatcgtcggcagggcttgggataacacaactgttgatctgagtggtgatgtgcaacagaatgcaaagcctgacaaaggtaactccaacagactcgctcaggcccaaggcatgcctgctggtttgacctactctcagacaatggaactcaaagacagcatgttgcaattggatccaaatgctaagacatggattgacatagaagggagacctgaagaccccgtggagatagctatctatcaacctaataatggtcagtatattcatttttacagggaaccaacagacattaaacaattcaaacaagactccaaacactctcatggcattgacatccaagacctattctcagttcagccggggttgacaagtgctgtaattgagagcctgccaaagaacatggtcttgtcgtgtcaaggtgctgatgacatcagaaagcttcttgactcccagaacaggagggacataaaactgattgatgtgtccatgcagaaagacgatgcaagaaaatttgaggataagatctgggatgaatacaaacacctttgtagaatgcatacggggattgtaacgcaaaagaagaagagaggtggcaaagaagaagtgacaccacactgtgcattgctggattgtctcatgtttgaagcagcagtcatagggagtccacaaattccaacccccagaccagtcttgagtagagacctggtgtttagaacaggtcctcccagagttgtcctgtaa